SEQUENCE LISTING (65 sequences)

SEQ ID NO: 1 - protein, 361 aa, synthetic construct
  VARIANT 1: note = Methionine may or may not be present
MHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFLHLQTHVFKRPMMIPKPVMFGTAFMSFFYVIIAFFKDIPDIEGDKDHGVKSLTMRLGQKRVFWICVSLLLTGYGAAIVVGATSSFLWCKLITVSGHALLASIFWNRAKSVDLKSHQEITSLYMFMWKLFYAEYFIIPLMR

SEQ ID NO: 2 - protein, 409 aa, synthetic construct
  VARIANT 1: note = Methionine may or may not be present
MASMFLGSLPLASSVNYIGRITRSKNCTESYHATSYITNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVVGATSSFLWCKLITVSGHALLASIFWNRAKSVDLKSHQEITSLYMFMWKLFYAEYFIIPLMR

SEQ ID NO: 3 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVVGATSSFLWCKLITVSGHALLASIFWNRAKSVDLKSHQEITSLYMFMWKLFYAEYFIIPLMR

SEQ ID NO: 4 - protein, 373 aa, synthetic construct
  VARIANT 1: note = Methionine may or may not be present
MTNASSNKTEKIKHEYANMRHRQHNLKHNYGGIEGVSTCEDWARNFVVNAASGFSLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVVGATSSFLWCKLITVSGHALLASIFWNRAKSVDLKSHQEITSLYMFMWKLFYAEYFIIPLMR

SEQ ID NO: 5 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWDSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVVGATSSFLWCKLITVSGHALLASIFWNRAKSVDLKSHQEITSLYMFMWKLFYAEYFIIPLMR

SEQ ID NO: 6 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSLDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVVGATSSFLWCKLITVSGHALLASIFWNRAKSVDLKSHQEITSLYMFMWKLFYAEYFIIPLMR

SEQ ID NO: 7 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKHGVTIITLCAILSLGVGWIVGSPPLFWSNFAYFVLGTVYSIDLPLMRWKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVVGATSSFLWCKLITVSGHALLASIFWNRAKSVDLKSHQEITSLYMFMWKLFYAEYFIIPLMR

SEQ ID NO: 8 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPVLFWSNFAYFVLGTVYSIDLPLMRWKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVVGATSSFLWCKLITVSGHALLASIFWNRAKSVDLKSHQEITSLYMFMWKLFYAEYFIIPLMR

SEQ ID NO: 9 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTGYSIDLPLMRWKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVVGATSSFLWCKLITVSGHALLASIFWNRAKSVDLKSHQEITSLYMFMWKLFYAEYFIIPLMR

SEQ ID NO: 10 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLLLMRWKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVVGATSSFLWCKLITVSGHALLASIFWNRAKSVDLKSHQEITSLYMFMWKLFYAEYFIIPLMR

SEQ ID NO: 11 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLVLMRWKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVVGATSSFLWCKLITVSGHALLASIFWNRAKSVDLKSHQEITSLYMFMWKLFYAEYFIIPLMR

SEQ ID NO: 12 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPYMRWKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVVGATSSFLWCKLITVSGHALLASIFWNRAKSVDLKSHQEITSLYMFMWKLFYAEYFIIPLMR

SEQ ID NO: 13 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRSKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVVGATSSFLWCKLITVSGHALLASIFWNRAKSVDLKSHQEITSLYMFMWKLFYAEYFIIPLMR

SEQ ID NO: 14 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRCKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVVGATSSFLWCKLITVSGHALLASIFWNRAKSVDLKSHQEITSLYMFMWKLFYAEYFIIPLMR

SEQ ID NO: 15 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRVKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVVGATSSFLWCKLITVSGHALLASIFWNRAKSVDLKSHQEITSLYMFMWKLFYAEYFIIPLMR

SEQ ID NO: 16 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRTKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVVGATSSFLWCKLITVSGHALLASIFWNRAKSVDLKSHQEITSLYMFMWKLFYAEYFIIPLMR

SEQ ID NO: 17 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRYKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVVGATSSFLWCKLITVSGHALLASIFWNRAKSVDLKSHQEITSLYMFMWKLFYAEYFIIPLMR

SEQ ID NO: 18 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRRKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVVGATSSFLWCKLITVSGHALLASIFWNRAKSVDLKSHQEITSLYMFMWKLFYAEYFIIPLMR

SEQ ID NO: 19 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRMKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVVGATSSFLWCKLITVSGHALLASIFWNRAKSVDLKSHQEITSLYMFMWKLFYAEYFIIPLMR

SEQ ID NO: 20 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRQKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVVGATSSFLWCKLITVSGHALLASIFWNRAKSVDLKSHQEITSLYMFMWKLFYAEYFIIPLMR

SEQ ID NO: 21 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGMTFHVGFFLHLQTHVFKRPMMIPKSVMFGTAFMSFFYVIIAFFKDIPDIEGDKDHGVKSLTMRLGQERVFWICVSLLLTGYGAAIVVGATSSFLWCKLITVSGHALLASIFWNRAKSVDLKSHQEITSLYMFMWKLFYAEYFIIPLMR

SEQ ID NO: 22 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFQHLQTHVFKRPMMIPKSVMFGTAFMSFFYVIIAFFKDIPDIEGDKDHGVKSLTMRLGQERVFWICVSLLLTGYGAAIVVGATSSFLWCKLITVSGHALLASIFWNRAKSVDLKSHQEITSLYMFMWKLFYAEYFIIPLMR

SEQ ID NO: 23 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFLHLQTHVFKRPMMIPKSVMFGTAEMSFFYVIIAFFKDIPDIEGDKDHGVKSLTMRLGQERVFWICVSLLLTGYGAAIVVGATSSFLWCKLITVSGHALLASIFWNRAKSVDLKSHQEITSLYMFMWKLFYAEYFIIPLMR

SEQ ID NO: 24 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFLHLQTHVFKRPMMIPKSVMFGTAFMSFFYVIIAFFRDIPDIEGDKDHGVKSLTMRLGQERVFWICVSLLLTGYGAAIVVGATSSFLWCKLITVSGHALLASIFWNRAKSVDLKSHQEITSLYMFMWKLFYAEYFIIPLMR

SEQ ID NO: 25 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFLHLQTHVFKRPMMIPKSVMFGTAFMSFFYVIIAFFKDIPDLEGDKDHGVKSLTMRLGQERVFWICVSLLLTGYGAAIVVGATSSFLWCKLITVSGHALLASIFWNRAKSVDLKSHQEITSLYMFMWKLFYAEYFIIPLMR

SEQ ID NO: 26 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFLHLQTHVFKRPMMIPKSVMFGTAFMSFFYVIIAFFKDIPDIEGDKCHGVKSLTMRLGQERVFWICVSLLLTGYGAAIVVGATSSFLWCKLITVSGHALLASIFWNRAKSVDLKSHQEITSLYMFMWKLFYAEYFIIPLMR

SEQ ID NO: 27 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFLHLQTHVFKRPMMIPKSVMFGTAFMSFFYVIIAFFKDIPDIEGDKKHGVKSLTMRLGQERVFWICVSLLLTGYGAAIVVGATSSFLWCKLITVSGHALLASIFWNRAKSVDLKSHQEITSLYMFMWKLFYAEYFIIPLMR

SEQ ID NO: 28 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFLHLQTHVFKRPMMIPKSVMFGTAFMSFFYVIIAFFKDIPDIEGDKRHGVKSLTMRLGQERVFWICVSLLLTGYGAAIVVGATSSFLWCKLITVSGHALLASIFWNRAKSVDLKSHQEITSLYMFMWKLFYAEYFIIPLMR

SEQ ID NO: 29 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFLHLQTHVFKRPMMIPKSVMFGTAFMSFFYVIIAFFKDIPDIEGDKMHGVKSLTMRLGQERVFWICVSLLLTGYGAAIVVGATSSFLWCKLITVSGHALLASIFWNRAKSVDLKSHQEITSLYMFMWKLFYAEYFIIPLMR

SEQ ID NO: 30 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFLHLQTHVFKRPMMIPKSVMFGTAFMSFFYVIIAFFKDIPDIEGDKLHGVKSLTMRLGQERVFWICVSLLLTGYGAAIVVGATSSFLWCKLITVSGHALLASIFWNRAKSVDLKSHQEITSLYMFMWKLFYAEYFIIPLMR

SEQ ID NO: 31 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFLHLQTHVFKRPMMIPKSVMFGTAFMSFFYVIIAFFKDIPDIEGDKDHGVKSLTVRLGQERVFWICVSLLLTGYGAAIVVGATSSFLWCKLITVSGHALLASIFWNRAKSVDLKSHQEITSLYMFMWKLFYAEYFIIPLMR

SEQ ID NO: 32 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFLHLQTHVFKRPMMIPKSVMFGTAFMSFFYVIIAFFKDIPDIEGDKDHGVKSLTFRLGQERVFWICVSLLLTGYGAAIVVGATSSFLWCKLITVSGHALLASIFWNRAKSVDLKSHQEITSLYMFMWKLFYAEYFIIPLMR

SEQ ID NO: 33 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFLHLQTHVFKRPMMIPKSVMFGTAFMSFFYVIIAFFKDIPDIEGDKDHGVKSLTYRLGQERVFWICVSLLLTGYGAAIVVGATSSFLWCKLITVSGHALLASIFWNRAKSVDLKSHQEITSLYMFMWKLFYAEYFIIPLMR

SEQ ID NO: 34 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAVVVGATSSFLWCKLITVSGHALLASIFWNRAKSVDLKSHQEITSLYMFMWKLFYAEYFIIPLMR

SEQ ID NO: 35 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVWGATSSFLWCKLITVSGHALLASIFWNRAKSVDLKSHQEITSLYMFMWKLFYAEYFIIPLMR

SEQ ID NO: 36 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVAGATSSFLWCKLITVSGHALLASIFWNRAKSVDLKSHQEITSLYMFMWKLFYAEYFIIPLMR

SEQ ID NO: 37 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVFGATSSFLWCKLITVSGHALLASIFWNRAKSVDLKSHQEITSLYMFMWKLFYAEYFIIPLMR

SEQ ID NO: 38 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVGGATSSFLWCKLITVSGHALLASIFWNRAKSVDLKSHQEITSLYMFMWKLFYAEYFIIPLMR

SEQ ID NO: 39 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVYGATSSFLWCKLITVSGHALLASIFWNRAKSVDLKSHQEITSLYMFMWKLFYAEYFIIPLMR

SEQ ID NO: 40 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVCGATSSFLWCKLITVSGHALLASIFWNRAKSVDLKSHQEITSLYMFMWKLFYAEYFIIPLMR

SEQ ID NO: 41 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVLGATSSFLWCKLITVSGHALLASIFWNRAKSVDLKSHQEITSLYMFMWKLFYAEYFIIPLMR

SEQ ID NO: 42 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVVIATSSFLWCKLITVSGHALLASIFWNRAKSVDLKSHQEITSLYMFMWKLFYAEYFIIPLMR

SEQ ID NO: 43 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVVGATSPFLWCKLITVSGHALLASIFWNRAKSVDLKSHQEITSLYMFMWKLFYAEYFIIPLMR

SEQ ID NO: 44 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVVGATSIFLWCKLITVSGHALLASIFWNRAKSVDLKSHQEITSLYMFMWKLFYAEYFIIPLMR

SEQ ID NO: 45 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVVGATSSRLWCKLITVSGHALLASIFWNRAKSVDLKSHQEITSLYMFMWKLFYAEYFIIPLMR

SEQ ID NO: 46 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVVGATSSGLWCKLITVSGHALLASIFWNRAKSVDLKSHQEITSLYMFMWKLFYAEYFIIPLMR

SEQ ID NO: 47 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVVGATSSFPWCKLITVSGHALLASIFWNRAKSVDLKSHQEITSLYMFMWKLFYAEYFIIPLMR

SEQ ID NO: 48 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVVGATSSFLDCKLITVSGHALLASIFWNRAKSVDLKSHQEITSLYMFMWKLFYAEYFIIPLMR

SEQ ID NO: 49 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVVGATSSFLWCKLIGVSGHALLASIFWNRAKSVDLKSHQEITSLYMFMWKLFYAEYFIIPLMR

SEQ ID NO: 50 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVVGATSSFLWCKLITVSGHALLASIFWNRAKGVDLKSHQEITSLYMFMWKLFYAEYFIIPLMR

SEQ ID NO: 51 - protein, 373 aa, synthetic construct
  VARIANT 1: note = Methionine may or may not be present
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVVGATSSFLWCKLITVSGHALLASIFWNRAKSVDLKSHQEITSFYMFMWKLFYAEYFIIPLMR

SEQ ID NO: 52 - protein, 669 aa, Arabidopsis thaliana
  VARIANT 1: note = Methionine may or may not be present
MARSFGANSTVVLAIIFFGCLFALSSAIEEATKLGSVIGIDLGTTYSCVGVYKNGHVEIIANDQGNRITPSWVGFTDSERLIGEAAKNQAAVNPERTVFDVKRLIGRKFEDKEVQKDRKLVPYQIVNKDGKPYIQVKIKDGETKVFSPEEISAMILTKMKETAEAYLGKKIKDAVVTVPAYFNDAQRQATKDAGVIAGLNVARIINEPTAAAIAYGLDKKGGEKNILVFDLGGGTFDVSVLTIDNGVFEVLSTNGDTHLGGEDFDHRVMEYFIKLIKKKHQKDISKDNKALGKLRRECERAKRALSSQHQVRVEIESLFDGVDFSEPLTRARFEELNNDLFRKTMGPVKKAMDDAGLQKSQIDEIVLVGGSTRIPKVQQLLKDFFEGKEPNKGVNPDEAVAYGAAVQGGILSGEGGDETKDILLLDVAPLTLGIETVGGVMTKLIPRNTVIPTKKSQVFTTYQDQQTTVSIQVFEGERSLTKDCRLLGKFDLNGIPPAPRGTPQIEVTFEVDANGILNVKAEDKASGKSEKITITNEKGRLSQEEIDRMVKEAEEFAEEDKKVKEKIDARNALETYVYNMKNQVNDKDKLADKLEGDEKEKIEAATKEALEWLDENQNSEKEEYDEKLKEVEAVCNPIITAVYQRSGGAPGGAGGESSTEEEDESHDEL

SEQ ID NO: 53 - protein, 643 aa, synthetic construct
  VARIANT 1: note = Methionine may or may not be present
MIEEATKLGSVIGIDLGTTYSCVGVYKNGHVEIIANDQGNRITPSWVGFTDSERLIGEAAKNQAAVNPERTVFDVKRLIGRKFEDKEVQKDRKLVPYQIVNKDGKPYIQVKIKDGETKVFSPEEISAMILTKMKETAEAYLGKKIKDAVVTVPAYFNDAQRQATKDAGVIAGLNVARIINEPTAAAIAYGLDKKGGEKNILVFDLGGGTFDVSVLTIDNGVFEVLSTNGDTHLGGEDFDHRVMEYFIKLIKKKHQKDISKDNKALGKLRRECERAKRALSSQHQVRVEIESLFDGVDFSEPLTRARFEELNNDLFRKTMGPVKKAMDDAGLQKSQIDEIVLVGGSTRIPKVQQLLKDFFEGKEPNKGVNPDEAVAYGAAVQGGILSGEGGDETKDILLLDVAPLTLGIETVGGVMTKLIPRNTVIPTKKSQVFTTYQDQQTTVSIQVFEGERSLTKDCRLLGKFDLNGIPPAPRGTPQIEVTFEVDANGILNVKAEDKASGKSEKITITNEKGRLSQEEIDRMVKEAEEFAEEDKKVKEKIDARNALETYVYNMKNQVNDKDKLADKLEGDEKEKIEAATKEALEWLDENQNSEKEEYDEKLKEVEAVCNPIITAVYQRSGGAPGGAGGESSTEEEDESHDEL

SEQ ID NO: 54 - protein, 642 aa, synthetic construct
  VARIANT 1: note = Methionine may or may not be present
MSKAVGIDLGTTYSCVAHFANDRVEIIANDQGNRTTPSYVAFTDTERLIGDAAKNQAAMNPHNTVFDAKRLIGRKFDDPEVTNDAKHYPFKVIDKGGKPVVQVEYKGETKTFTPEEISSMILTKMKETAENFLGTEVKDAVVTVPAYFNDSQRQATKDAGTIAGLNVLRIINEPTAAAIAYGLDKKSQKEHNVLIFDLGGGTFDVSLLSIDEGVFEVKATAGDTHLGGEDFDSRLVNFLAEEFKRKNKKDLTTNQRSLRRLRTAAERAKRTLSSSAQTSIEIDSLFEGIDFYTSITRARFEELCADLFRSTLEPVEKVLADSKLDKSQIDEIVLVGGSTRIPKVQKLVSDFFNGKEPNRSINPDEAVAYGAAVQAAILTGDQSSTTQDLLLLDVAPLSLGIETAGGIMTKLIPRNSTIPTKKSEVFSTYADNQPGVLIQVFEGERTRTKDNNLLGKFELSGIPPAPRGVPQIEVTFDIDANGILNVSAVEKGTGKSNKITITNDKGRLSKEDIDKMVAEAEKFKAEDEQEAQRVQAKNQLESYAFTLKNSVSENNFKEKVGEEDAKKLEAAAQDAINWLDASQAASTEEYKERQKELEGVANPIMSKFYGAAGGAPGAGPVPGAGAGPTGAPDNGPTVEEVD

SEQ ID NO: 55 - protein, 682 aa, synthetic construct
  VARIANT 1: note = Methionine may or may not be present
MFFNRLSAGKLLVPLSVVLYALFVVILPLQNSFHSSNVLVRGADDVENYGTVIGIDLGTTYSCVAVMKNGKTEILANEQGNRITPSYVAFTDDERLIGDAAKNQVAANPQNTIFDIKRLIGLKYNDRSVQKDIKHLPFNVVNKDGKPAVEVSVKGEKKVFTPEEISGMILGKMKQIAEDYLGTKVTHAVVTVPAYFNDAQRQATKDAGTIAGLNVLRIVNEPTAAAIAYGLDKSDKEHQIIVYDLGGGTFDVSLLSIENGVFEVQATSGDTHLGGEDFDYKIVRQLIKAFKKKHGIDVSDNNKALAKLKREAEKAKRALSSQMSTRIEIDSFVDGIDLSETLTRAKFEELNLDLFKKTLKPVEKVLQDSGLEKKDVDDIVLVGGSTRIPKVQQLLESYFDGKKASKGINPDEAVAYGAAVQAGVLSGEEGVEDIVLLDVNALTLGIETTGGVMTPLIKRNTAIPTKKSQIFSTAVDNQPTVMIKVYEGERAMSKDNNLLGKFELTGIPPAPRGVPQIEVTFALDANGILKVSATDKGTGKSESITITNDKGRLTQEEIDRMVEEAEKFASEDASIKAKVESRNKLENYAHSLKNQVNGDLGEKLEEEDKETLLDAANDVLEWLDDNFETAIAEDFDEKFESLSKVAYPITSKLYGGADGSGAADYDDEDEDDDGDYFEHDEL

SEQ ID NO: 56 - protein, 401 aa, synthetic construct
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVVGATSSFLWCKLITVSGHALLASIFWNRAKSVDLKSHQEITSLYMFMWKLFYAEYFIIPLMRILEQPLKFVLTAAVVLLTTSVLCCVVFT

SEQ ID NO: 57 - protein, 405 aa, synthetic construct
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVVGATSSFLWCKLITVSGHALLASIFWNRAKSVDLKSHQEITSLYMFMWKLFYAEYFIIPLMRWYKDLKMKMCLALVIIILLVVIIVPIAVHFSR

SEQ ID NO: 58 - protein, 376 aa, synthetic construct
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVVGATSSFLWCKLITVSGHALLASIFWNRAKSVDLKSHQEITSLYMFMWKLFYAEYFIIPLMRSKL

SEQ ID NO: 59 - protein, 408 aa, synthetic construct
MTNASSNKTEKIKHEYANMRHRQHNLKHNYGGIEGVSTCEDWARNFVVNAASGESLESHEAQHHTPETLWGSIKQFCDAFYRFSRPHVIIGTAVNIIVMSSLALEKSSDISPKFFIGLFQVIVTILSMNIYTAGINQLTDIEIDKINKPYLPLASGEYSYKTGVTIITLCAILSLGVGWIVGSPPLFWSNFAYFVLGTVYSIDLPLMRWKSHPALAALFFFVIRGLTFHVGFFLHLQTHVFKRPMMIPKSVMFGTAFMSFFYVIIAFFKDIPDIEGDKDHGVKSLTMRLGQERVFWICVSLLLTGYGAAIVVGATSSFLWCKLITVSGHALLASIFWNRAKSVDLKSHQEITSLYMFMWKLFYAEYFIIPLMRNIKEIMWWQKVKNITLLTFTIILFVSAAFMFFYLW

SEQ ID NO: 60 - protein, 669 aa, synthetic construct; identical to SEQ ID NO: 52
  VARIANT 2..27: note = This region may have 2-27 amino acids deleted from the N-terminus

SEQ ID NO: 61 - protein, 8 aa, synthetic construct
ACFGIMTV

SEQ ID NO: 62 - protein, 7 aa, synthetic construct
CDEKPQS

SEQ ID NO: 63 - protein, 6 aa, synthetic construct
AEMRST

SEQ ID NO: 64 - protein, 10 aa, synthetic construct
FHIKLPQRWY

SEQ ID NO: 65 - protein, 643 aa, synthetic construct; identical to SEQ ID NO: 53

(19) United States
(12) Patent Application Publication
(10) Pub. No.: US 2024/0101614 A1
(43) Pub. Date: Mar. 28, 2024

(54) CHAPERONES FOR HETEROLOGOUS EXPRESSION SYSTEMS

(71) Applicant: Inscripta, Inc., Pleasanton, CA (US)

(72) Inventors: Amanda Reider Apel, Alameda, CA (US); Karolina Kalbarczyk, Santa Cruz, CA (US); Drew Fraser Thacker, Alameda, CA (US)

(73) Assignee: Inscripta, Inc., Pleasanton, CA (US)

(21) Appl. No.: 18/455,221

(22) Filed: Aug. 24, 2023

Related U.S. Application Data
(60) Provisional application No. 63/401,064, filed on Aug. 25, 2022.

(51) Int. Cl. C07K 14/415; C12N 9/10

(52) U.S. Cl. CPC C07K 14/415; C12N 9/1085

(57) ABSTRACT

The present disclosure relates to synthetic biology and, in particular, the expression of heterologous proteins in a microbial cell, and engineered enzymes for achieving the same.

### CROSS-REFERENCE TO RELATED APPLICATIONS

The present application claims priority to U.S. Provisional Application No. 63/401,064, filed Aug. 25, 2022, the entire contents of which are incorporated herein by reference.

### SEQUENCE LISTING

The instant application contains a Sequence Listing which has been submitted electronically in XML format and is hereby incorporated by reference in its entirety. Said XML copy, created on Oct. 20, 2022, is named 131881-0112_IP-1011-PRV1_SL.xml and is 103,196 bytes in size.

### BACKGROUND

The following discussion is merely provided to aid the reader in understanding the disclosure and is not admitted to describe or constitute prior art thereto.

Heterologous expression of proteins in microbial systems is important to synthetic biology. However, expression of many proteins of interest can be hindered as a result of improper protein folding. Not only are misfolded proteins generally non-functional, proteins that fold improperly may also impact the health of the cell regardless of the function of the protein. Thus, when proteins fail to fold into their functional state, the resulting misfolded proteins can be contorted into shapes that are unfavorable to the crowded cellular environment.

To avoid these issues, cells in nature often express a complex network of molecular chaperones, which use various mechanisms to prevent aggregation and promote efficient folding. Because protein molecules are highly dynamic, constant chaperone surveillance may be involved to ensure protein homeostasis and proper folding. Nevertheless, existing chaperones in various microbial expression systems may be insufficient to ensure consistent proper protein folding of various heterologous proteins of interest.

### SUMMARY

The present disclosure provides chaperones that aid in the proper folding of heterologous proteins, microbial cells (e.g., bacteria and yeast) that express heterologous protein(s) of interest, and methods of co-expressing the chaperones disclosed herein with a heterologous protein or interest, as well as methods of improving heterologous protein folding. The disclosed compositions, systems, and methods may be used to improve bioproduction of various compounds of interest, such as terpenes or isoprenoids.

In one aspect, the present disclosure provides engineered chaperone proteins comprising an amino acids sequence in which 2-27 amino acids are deleted from the N-terminus of

(SEQ ID NO: 60)
MARSFGANSTVVLAIIFFGCLFALSSAIEEATKLGSVIGIDLGTTYSCVG
VYKNGHVEIIANDQGNRITPSWVGFTDSERLIGEAAKNQAAVNPERTVFD
VKRLIGRKFEDKEVQKDRKLVPYQIVNKDGKPYIQVKIKDGETKVFSPEE
ISAMILTKMKETAEAYLGKKIKDAVVTVPAYFNDAQRQATKDAGVIAGLN
VARIINEPTAAAIAYGLDKKGGEKNILVFDLGGGTFDVSVLTIDNGVFEV
LSTNGDTHLGGEDFDHRVMEYFIKLIKKKHQKDISKDNKALGKLRRECER
AKRALSSQHQVRVEIESLFDGVDFSEPLTRARFEELNNDLFRKTMGPVKK
AMDDAGLQKSQIDEIVLVGGSTRIPKVQQLLKDFFEGKEPNKGVNPDEAV
AYGAAVQGGILSGEGGDETKDILLLDVAPLTLGIETVGGVMTKLIPRNTV
IPTKKSQVFTTYQDQQTTVSIQVFEGERSLTKDCRLLGKFDLNGIPPAPR
GTPQIEVTFEVDANGILNVKAEDKASGKSEKITITNEKGRLSQEEIDRMV
KEAEEFAEEDKKVKEKIDARNALETYVYNMKNQVNDKDKLADKLEGDEKE
KIEAATKEALEWLDENQNSEKEEYDEKLKEVEAVCNPIITAVYQRSGGAP
GGAGGESSTEEEDESHDEL.

In some implementations, the protein comprises an N-terminal deletion of 27 amino acids from SEQ ID NO: 52.

In some implementations, the protein has an amino acid sequence consisting of:

(SEQ ID NO: 65)
MIEEATKLGSVIGIDLGTTYSCVGVYKNGHVEIIANDQGNRITPSWVGFT
DSERLIGEAAKNQAAVNPERTVFDVKRLIGRKFEDKEVQKDRKLVPYQIV
NKDGKPYIQVKIKDGETKVFSPEEISAMILTKMKETAEAYLGKKIKDAVV
TVPAYFNDAQRQATKDAGVIAGLNVARIINEPTAAAIAYGLDKKGGEKNI
LVFDLGGGTFDVSVLTIDNGVFEVLSTNGDTHLGGEDFDHRVMEYFIKLI
KKKHQKDISKDNKALGKLRRECERAKRALSSQHQVRVEIESLFDGVDFSE
PLTRARFEELNNDLFRKTMGPVKKAMDDAGLOKSQIDEIVLVGGSTRIPK
VQQLLKDFFEGKEPNKGVNPDEAVAYGAAVQGGILSGEGGDETKDILLLD
VAPLTLGIETVGGVMTKLIPRNTVIPTKKSQVFTTYQDQQTTVSIQVFEG
ERSLTKDCRLLGKFDLNGIPPAPRGTPQIEVTFEVDANGILNVKAEDKAS
GKSEKITITNEKGRLSQEEIDRMVKEAEEFAEEDKKVKEKIDARNALETY
VYNMKNQVNDKDKLADKLEGDEKEKIEAATKEALEWLDENQNSEKEEYDE
KLKEVEAVCNPIITAVYQRSGGAPGGAGGESSTEEEDESHDEL.

In some implementations, the protein exhibits protein-folding activity.

In some implementations, the protein exhibits increased protein-folding activity relative to a wild-type form of the protein.

In another aspect, the present disclosure provides engineered microbial cells that express a heterologous chaperone protein or variant thereof, wherein the heterologous protein comprises an amino acid sequence that has least about 65% sequence identity to SEQ ID NO: 52, SEQ ID NO: 53, SEQ ID NO: 54, or SEQ ID NO: 55.

In some implementations, the protein or variant thereof comprises an amino acid sequence that has at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 52, SEQ ID NO: 53, SEQ ID NO: 54, or SEQ ID NO: 55.

In some implementations, the protein or variant thereof comprises, or consists, of SEQ ID NO: 52.

In some implementations, the protein or variant thereof is a variant of SEQ ID NO: 1 comprising an N-terminal deletion of 1-27 amino acids from SEQ ID NO: 52.

In some implementations, the protein or variant thereof consists of SEQ ID NO: 53.

In some implementations, the protein or variant thereof comprises, or consists, of SEQ ID NO: 54.

In some implementations, the protein or variant thereof comprises, or consists, of SEQ ID NO: 55.

In some implementations, the microbial cell is a yeast cell or a bacterial cell.

In some implementations, the microbial cell expresses a second heterologous protein. In some implementations, the second heterologous protein is an enzyme. In some implementations, the enzyme catalyzes production of bakuchiol, exhibits prenyltransferase activity, or both.

In another aspect, the present disclosure provides methods of expressing a heterologous protein in a microbial cell, comprising co-expressing the heterologous protein and a heterologous chaperone protein.

In some implementations, the microbial cell is a yeast cell or a bacterial cell.

In some implementations, the heterologous chaperone protein is a chaperone protein from Arabidopsis thaliana.

In some implementations, the heterologous chaperone protein is Arabidopsis thaliana BIP1 (AtBIP1) or a variant thereof.

In some implementations, the heterologous chaperone protein comprises an amino acid sequence that has at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 52 or SEQ ID NO: 53.

In some implementations, the heterologous chaperone protein comprises, or consists, of SEQ ID NO: 52. In some implementations, the heterologous chaperone protein is a variant of SEQ ID NO: 32 comprising an N-terminal deletion of 1-27 amino acids from SEQ ID NO: 52. In some implementations, the heterologous chaperone protein consists of SEQ ID NO: 53.

In some implementations, the heterologous chaperone protein comprises an amino acid sequence that has at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 54 or SEQ ID NO: 55.

In some implementations, the heterologous chaperone protein comprises, or consists, of SEQ ID NO: 54.

In some implementations, the heterologous chaperone protein comprises, or consists, of SEQ ID NO: 55.

In some implementations, the heterologous protein is an enzyme. In some implementations, the enzyme catalyzes production of bakuchiol, exhibits prenyltransferase activity, or both. In some implementations, expression of the heterologous protein is increased relative to expression of the heterologous protein in a microbial cell that does not co-express the heterologous chaperone protein.

In another aspect, the present disclosure provides methods of facilitating folding of a heterologous protein in a microbial cell, the method comprising co-expressing in the microbial cell the heterologous protein and a heterologous chaperone protein, wherein the heterologous chaperone protein comprises an amino acid sequence that has at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 52, SEQ ID NO: 53, SEQ ID NO: 54, or SEQ ID NO: 55.

In some implementations, the microbial cell is a yeast cell or a bacterial cell.

In some implementations, expression of the heterologous protein is enhanced relative to expression of the heterologous protein in a microbial cell that does not co-express the heterologous chaperone protein.

In some implementations, the heterologous protein misfolds when expressed in a microbial cell that does not co-express the heterologous chaperone protein.

In some implementations, the protein or variant thereof comprises, or consists, of SEQ ID NO: 52.

In some implementations, the protein or variant thereof is a variant of SEQ ID NO: 1 comprising an N-terminal deletion of 1-27 amino acids from SEQ ID NO: 52.

In some implementations, the protein or variant thereof consists of SEQ ID NO: 53.

In some implementations, the protein or variant thereof comprises, or consists, of SEQ ID NO: 54.

In some implementations, the protein or variant thereof comprises, or consists, of SEQ ID NO: 55.

In some implementations, the heterologous protein is an enzyme. In some implementations, the enzyme catalyzes production of bakuchiol, exhibits prenyltransferase activity, or both.

In another aspect, the present disclosure provides engineered enzymes comprising an N-terminal deletion of: 1 to about 73 amino acids from the N-terminus of SEQ ID NO: 1 or 1 to about 120 amino acids from the N-terminus of SEQ ID NO: 2.

In some implementations, the enzyme comprises an N-terminal deletion of 29, 57, or 73 amino acids from the N-terminus of SEQ ID NO: 1.

In some implementations, the enzyme comprises an N-terminal deletion of 38, 88, 105, or 120 amino acids from the N-terminus of SEQ ID NO: 2.

In some implementations, the enzyme comprises an amino acid sequence having at least about 80%, at least about 85%, at least about 90%, at least about 95%, or 100% identity to the amino acid sequence set forth in SEQ ID NO: 3. In some implementations, the engineered enzyme comprises at least one amino acid substitution at position 54, 71, 108, 162, 185, 199, 205, 206, 209, 226, 234, 257, 269, 274, 279, 287, 310, 312, 313, 317, 318, 319, 320, 325, 342, and 354 of SEQ ID NO: 3. In some implementations, the enzyme comprises at least one amino acid substitution, relative to SEQ ID NO: 3, selected from the group consisting of E54F, G71D, S108L, T162H, P185V, V199G, P205L, P205V, L206Y, W209S, W209C, W209V, W209T, W209Y, W209R, W209M, W209Q, L226M, L234Q, F257E, K269R, I274L, D279C, D279K, D279R, D279M, D279L, M287V, M287F, M287Y, I310V, V312W, V312A, V312F, V312G, V312Y, V312C, V312L, G313I, S317P, S317I, F318R, F318G, L319P, W320D, T325G, S342G, and L354F.

In some implementations, the enzyme comprises an amino acid sequence having at least 80%, at least 85%, at least 95%, or 100% identity to an amino acid sequence selected from the group consisting of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, and SEQ ID NO: 51.

In another aspect, the present disclosure provides engineered bakuchiol-producing enzymes, comprising an N-terminal deletion of 1 to about 120 amino acids from the N-terminus of the enzyme, wherein the enzyme catalyzes production of bakuchiol, exhibits prenyltransferase activity, or both.

In some implementations, the enzyme comprises an amino acid sequence with at least about 65% identity to SEQ ID NO: 1 or SEQ ID NO: 2.

In some implementations, the N-terminal deletion increases catalyzation of production of bakuchiol, prenyltransferase activity, or both, relative to a non-engineered enzyme comprising a same amino acid sequence but without the N-terminal deletion.

In some implementations, the enzyme comprises an N-terminal deletion of 29, 57, or 73 amino acids from the N-terminus of SEQ ID NO: 1. In some implementations, the enzyme comprises an N-terminal deletion of 39, 88, 105, or 120 amino acids from the N-terminus of SEQ ID NO: 2.

In some implementations, the enzyme comprises an amino acid sequence with at least about 65% identity to SEQ ID NO: 3. In some implementations, the engineered enzyme comprises at least one amino acid substitution, relative to SEQ ID NO: 3, at position 54, 71, 108, 162, 185, 199, 205, 206, 209, 226, 234, 257, 269, 274, 279, 287, 310, 312, 313, 317, 318, 319, 320, 325, 342, or 354. In some implementations, the enzyme comprises at least one amino acid substitution, relative to SEQ ID NO: 3, selected from the group consisting of E54F, G71D, S108L, T162H, P185V, V199G, P205L, P205V, L206Y, W209S, W209C, W209V, W209T, W209Y, W209R, W209M, W209Q, L226M, L234Q, F257E, K269R, I274L, D279C, D279K, D279R, D279M, D279L, M287V, M287F, M287Y, I310V, V312W, V312A, V312F, V312G, V312Y, V312C, V312L, G313I, S317P, S317I, F318R, F318G, L319P, W320D, T325G, S342G, and L354F.

In some implementations, the enzyme comprises an amino acid sequence having at least 80%, at least 85%, at least 95%, or 100% identity to an amino acid sequence selected from the group consisting of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, and SEQ ID NO: 51.

In another aspect, the present disclosure provides engineered enzymes comprising an amino acid sequence that is a variant of SEQ ID NO: 1, wherein the amino acid sequence comprises at least one substitution mutation relative to SEQ ID NO: 1 at one or more amino acid positions selected from 42, 59, 96, 150, 173, 187, 193, 194 197, 214, 222, 245, 257, 262, 267, 275, 298, 300, 301, 305, 306, 307, 308, 313, 330, and 342.

In another aspect, the present disclosure provides engineered enzymes comprising an amino acid sequence that is a variant of SEQ ID NO: 2, wherein the amino acid sequence comprises at least one substitution mutation relative to SEQ ID NO: 2 at one or more amino acid positions selected from 90, 107, 144, 198, 221, 235, 241, 242, 245, 262, 270, 293, 305, 310, 315, 323, 346, 348, 349, 353, 354, 355, 356, 361, 378, and 390.

In another aspect, the present disclosure provides engineered enzymes that catalyze production of bakuchiol, exhibits prenyltransferase activity, or both, wherein the engineered enzyme comprises at least one substitution mutation selected from: (a) substitution of a glutamate (E) corresponding to the E at position 42 of SEQ ID NO: 1 or position 90 of SEQ ID NO: 2; (b) substitution of a glycine (G) corresponding to the G at position 59 of SEQ ID NO: 1 or position 107 of SEQ ID NO: 2; (c) substitution of a serine (S) corresponding to the S at position 96 of SEQ ID NO: 1 or position 144 of SEQ ID NO: 2; (d) substitution of threonine (T) corresponding to the T at position 150 of SEQ ID NO: 1 or position 198 of SEQ ID NO: 2; (e) substitution of proline (P) corresponding to the P at position 173 of SEQ ID NO: 1 or position 221 of SEQ ID NO: 2; (f) substitution of valine (V) corresponding to the V at position 187 of SEQ ID NO: 1 or position 235of SEQ ID NO: 2; (g) substitution of proline (P) corresponding to the P at position 193 of SEQ ID NO: 1 or position 241 of SEQ ID NO: 2; (h) substitution of leucine (L) corresponding to the L at position 194 of SEQ ID NO: 1 or position 242 of SEQ ID NO: 2; (i) substitution of tryptophan (W) corresponding to the W at position 197 of SEQ ID NO: 1 or position 245 of SEQ ID NO: 2; (j) substitution of leucine (L) corresponding to the L at position 214 of SEQ ID NO: 1 or position 262 of SEQ ID NO: 2; (k) substitution of leucine (L) corresponding to the L at position 222 of SEQ ID NO: 1 or position 270 of SEQ ID NO: 2; (l) substitution of phenylalanine (F) corresponding to the F at position 245 of SEQ ID NO: 1 or position 293 of SEQ ID NO: 2; (m) substitution of lysine (K) corresponding to the K at position 257 of SEQ ID NO: 1 or position 305 of SEQ ID NO: 2; (n) substitution of isoleucine (I) corresponding to the I at position 262 of SEQ ID NO: 1 or position 310 of SEQ ID NO: 2; (o) substitution of aspartic acid (D) corresponding to the D at position 267 of SEQ ID NO: 1 or position 315 of SEQ ID NO: 2; (p) substitution of methionine (M) corresponding to the M at position 275 of SEQ ID NO: 1 or position 323 of SEQ ID NO: 2; (q) substitution of isoleucine (I) corresponding to the I at position 298 of SEQ ID NO: 1 or position 346 of SEQ ID NO: 2; (r) substitution of valine (V) corresponding to the V at position 300 of SEQ ID NO: 1 or position 348 of SEQ ID NO: 2; (s) substitution of glycine (G) corresponding to the G at position 301 of SEQ ID NO: 1 or position 349 of SEQ ID NO: 2; (t) substitution of serine (S) corresponding to the S at position 305 of SEQ ID NO: 1 or position 353 of SEQ ID NO: 2; (u) substitution of phenylalanine (F) corresponding to the F at position 306 of SEQ ID NO: 1 or position 354 of SEQ ID NO: 2; (v) substitution of leucine (L) corresponding to the L at position 307 of SEQ ID NO: 1 or position 355 of SEQ ID NO: 2; (w) substitution of tryptophan (W) corresponding to the W at position 308 of SEQ ID NO: 1 or position 356 of SEQ ID NO: 2; (x) substitution of threonine (T) corresponding to the T at position 313 of SEQ ID NO: 1 or position 361 of SEQ ID NO: 2; (y) substitution of serine (S) corresponding to the S at position 330 of SEQ ID NO: 1 or position 378 of SEQ ID NO: 2; and (z) substitution of leucine (L) corresponding to the L at position 342 of SEQ ID NO: 1 or position 390 of SEQ ID NO: 2.

In another aspect, the present disclosure provides engineered enzymes that catalyze production of bakuchiol, exhibits prenyltransferase activity, or both, wherein the engineered enzyme comprises at least one substitution mutation selected from: (a) substitution of phenylalanine (F) at position 42 of SEQ ID NO: 1 or position 90 of SEQ ID NO: 2; (b) substitution of aspartate (D) at position 59 of SEQ ID NO: 1 or position 107 of SEQ ID NO: 2; (c) substitution of leucine (L) at position 96 of SEQ ID NO: 1 or position 144 of SEQ ID NO: 2; (d) substitution of histidine (H) at position 150 of SEQ ID NO: 1 or position 198 of SEQ ID NO: 2; (e) substitution of valine (V) at position 173 of SEQ ID NO: 1 or position 221 of SEQ ID NO: 2; (f) substitution of glycine (G) at position 187 of SEQ ID NO: 1 or position 235 of SEQ ID NO: 2; (g) substitution of leucine (L) or valine (V) at position 193 of SEQ ID NO: 1 or position 241 of SEQ ID NO: 2; (h) substitution of tyrosine (Y) at position 194 of SEQ ID NO: 1 or position 242 of SEQ ID NO: 2; (i) substitution of serine (S), cysteine (C), valine (V), threonine (T), tyrosine (Y), arginine (R), methionine (M), or glutamine (Q) at position 197 of SEQ ID NO: 1 or position 245 of SEQ ID NO: 2; (j) substitution of methionine (M) at position 214 of SEQ ID NO: 1 or position 262 of SEQ ID NO: 2; (k) substitution of glutamine (Q) at position 222 of SEQ ID NO: 1 or position 270 of SEQ ID NO: 2; (l) substitution of glutamate (E) at position 245 of SEQ ID NO: 1 or position 293 of SEQ ID NO: 2; (m) substitution of arginine (R) at position 257 of SEQ ID NO: 1 or position 305 of SEQ ID NO: 2; (n) substitution of leucine (L) at position 262 of SEQ ID NO: 1 or position 310 of SEQ ID NO: 2; (o) substitution of cysteine (C), lysine (K), arginine (R), methionine (M), or leucine (L) at position 267 of SEQ ID NO: 1 or position 315 of SEQ ID NO: 2; (p) substitution of valine (V), phenylalanine (F), or tyrosine (Y) at position 275 of SEQ ID NO: 1 or position 323 of SEQ ID NO: 2;(q) substitution of valine (V) at position 298 of SEQ ID NO: 1 or position 346 of SEQ ID NO: 2; (r) substitution of tryptophan (W), alanine (A), phenylalanine (F), glycine (G), tyrosine (Y), cysteine (C), or leucine (L) at position 300 of SEQ ID NO: 1 or position 348 of SEQ ID NO: 2; (s) substitution of isoleucine (I) at position 301 of SEQ ID NO: 1 or position 349 of SEQ ID NO: 2; (t) substitution of proline (P) or isoleucine (I) at position 305 of SEQ ID NO: 1 or position 353 of SEQ ID NO: 2; (u) substitution of arginine (R) or glycine (G) at position 306 of SEQ ID NO: 1 or position 354 of SEQ ID NO: 2; (v) substitution of proline (P) at position 307 of SEQ ID NO: 1 or position 355 of SEQ ID NO: 2; (w) substitution of aspartate (D) at position 308 of SEQ ID NO: 1 or position 356 of SEQ ID NO: 2; (x) substitution of glycine (G) at position 313 of SEQ ID NO: 1 or position 361 of SEQ ID NO: 2; (y) substitution of glycine (G) at position 330 of SEQ ID NO: 1 or position 378 of SEQ ID NO: 2; and (z) substitution of phenylalanine (F) at position 342 of SEQ ID NO: 1 or position 390 of SEQ ID NO: 2.

In some implementations, a substitution mutation in the disclosed engineered enzymes increases catalyzation of production of bakuchiol, prenyltransferase activity, or both, relative to a non-engineered enzyme comprising the same amino acid sequence but without the substitution mutation.

In some implementations, the disclosed engineered enzymes may further comprise an N-terminal deletion of 1-120 amino acids.

In another aspect, the present disclosure provides engineered enzymes, comprising an amino acid sequence that is a variant of SEQ ID NO: 3, wherein the amino acid sequence comprises at least one substitution mutation relative to SEQ ID NO: 3 at one or more amino acid positions selected from 54, 71, 108, 162, 185, 199, 205, 206, 209, 226, 234, 257, 269, 274, 279, 287, 310, 312, 313, 317, 318, 319, 320, 325, 342, and 354.

In another aspect, the present disclosure provides engineered enzymes that catalyze production of bakuchiol, exhibits prenyltransferase activity, or both, the enzyme comprising nine transmembrane domains and loops connecting the transmembrane domains, wherein the enzyme comprises at least one substitution mutation on an internal loop or an external loop of the enzyme.

In some implementations, the enzyme comprises an N-terminus and a C-terminus, and no amino acids are substituted in the first 50 amino acids of the N-terminus or the terminal 50 amino acids of the C-terminus.

In some implementations, the substitution mutation increases catalyzation of production of bakuchiol, prenyltransferase activity, or both, relative to a non-engineered enzyme comprising the same amino acid sequence but without the substitution mutation.

In another aspect, the present disclosure provides transgenic cells, comprising a transgene encoding an engineered enzyme disclosed herein.

In some implementations, the transgenic cell is prokaryotic. In some implementations, the transgenic cell is selected from Escherichia coli (E. coli), an Acinetobacter species, a Pseudomonas species, a Streptomyces species, a Bacillus species, and a Mycobacterium species.

In some implementations, the transgenic cell is eukaryotic. In some implementations, the transgenic cell is selected from a yeast species, a filamentous fungus, an algae, and an amoeba. In some implementations, the filamentous fungus is selected from an Aspergillus species and a Trichoderma species. In some implementations, the amoeba is Dictyostelium discoideum. In some implementations, the algae is selected from Botryococcus braunii, Chlorella sp., Crypthecodinium cohnii, Cylindrotheca sp., Nitzschia sp., Phaeodactylum tricornutum, Schizochytrium sp., and Tetraselmis suecia. In some implementations, the yeast species is Saccharomyces cerevisiae (S. cerevisiae), Pichia pastoris, or Kluyveromyces marxianus. In some implementations, the yeast species is an oleaginous yeast.

In some implementations, the transgene is integrated into the transgenic cell's genome. In some implementations, the transgene is not integrated into the transgenic cell's genome.

In some implementations, the engineered enzyme comprises an amino acid sequence selected from any one of SEQ ID NOs: 1-51. In some implementations, the engineered enzyme has an amino acid sequence consisting of any one of SEQ ID NOs: 1-51.

In another aspect, the present disclosure provides methods of producing bakuchiol, comprising culturing the transgenic cell disclosed herein in a culture medium and in the presence of p-coumaric acid and (i) geranyl pyrophosphate (GPP), (ii) dimethylallyl pyrophosphate (DMAPP), (iii) isopentenyl pyrophosphate (IPP), or any combination of (i)-(iii).

In another aspect, the present disclosure provides a bioproduction batch of bakuchiol produced by the methods disclosed herein.

In another aspect, the present disclosure provides a nucleic acid comprising a nucleic acid sequence encoding an engineered enzyme disclosed herein.

In another aspect, the present disclosure provides an engineered host cell that produces an engineered enzyme disclosed herein or that comprises a nucleic acid disclosed herein.

In another aspect, the present disclosure provides a bakuchiol-producing enzyme as disclosed herein. In another aspect, the present disclosure provides a transgenic cell capable of producing bakuchiol as disclosed herein. In another aspect, the present disclosure provides a method of producing bakuchiol as disclosed herein.

The foregoing general description and following detailed description are examples and are intended to provide further explanation of the disclosure as claimed. Other objects, advantages, and novel features will be readily apparent to those skilled in the art from the following brief description of the drawings and detailed description of the disclosure.

It should be appreciated that all combinations of the foregoing concepts and additional concepts discussed in greater detail below are provided as being part of the inventive subject matter disclosed herein and may be employed in any combination to achieve the benefits described herein.

### BRIEF DESCRIPTION OF DRAWINGS

FIG. 1 (FIG. 1) shows, in one implementation, an illustration of the predicted protein structures of BAK28 and BAK36 generated using AlphaFold.

FIG. 2 (FIG. 2) shows, in one implementation, a graph of bakuchiol titers achieved by various BAK28 and BAK36 N-terminal truncation mutants relative to their respective full-length parent strains. STR764/Parent (ARS1206::pCCW12>ERG20(F96W_N127W_K197G)-tPRM9); STR882 (GAL80\hat{ }::pGAL1>BAK28-tGAT2); STR1292 (T1 deletion; GAL80\hat{ }::pGAL1>BAK28(T1)-tGAT2); STR1293: (T2 deletion; GAL80\hat{ }::pGAL1>BAK28(T2)-tGAT2); STR 1294 (T3 deletion; GAL80\hat{ }::pGAL1>BAK28(T3)-tGAT2); STR890/Parent (GAL80\hat{ }::pGAL1>BAK36-tGAT2); STR1288 (T1 deletion; GAL80\hat{ }::pGAL1>BAK36(T1)-tGAT2); STR1291 (T2 deletion; GAL80\hat{ }::pGAL1>BAK36(T2)-tGAT2); STR1290 (T3 deletion; GAL80\hat{ }::pGAL1>BAK36(T3)-tGAT2); STR1289 (T4 deletion; GAL80\hat{ }::pGAL1>BAK36(T4)-tGAT2).

FIG. 3 (FIG. 3) shows, in one implementation, an illustration of the predicted protein structure of BAK36 generated using AlphaFold with residues V199, P205, and W209 highlighted. Changes in these residues increased activity.

FIG. 4 (FIG. 4) shows, in one implementation, an illustration of the predicted protein structure of BAK36 generated using AlphaFold, with several residues highlighted. The residues identified in this figure either (a) decreased activity, or (b) increased activity with some substitutions but decreased activity with other substitutions. Figure discloses SEQ ID NOS 61-64, respectively, in order of appearance.

FIG. 5 (FIG. 5) shows, in one implementation, a graph illustrating the effect of various C-terminal tags on bakuchiol production by BAK36 (T1).

FIG. 6 (FIG. 6) shows, in one implementation, a set of graphs illustrating the effect of heterologous chaperone co-expression on bakuchiol production by BAK36 (T1).

### DETAILED DESCRIPTION

Expression of heterologous proteins in microbial systems such as yeast and bacteria is challenging, particularly for expression of heterologous transmembrane proteins. This difficulty may arise, at least in part, due to the fact that heterologous proteins may not fold properly in a microbial expression system. Misfolded proteins may be inactive or even toxic. This type of improper folding is a continuing challenge in the fields of synthetic biology and bioproduction, but the present disclosure provides solutions to the challenges and the benefits of chaperones and microbial expression systems for enhancing or increasing expression of heterologous proteins and decreasing the risk of misfolding of heterologous proteins.

### I. Definitions

It is to be understood that the disclosed compositions and methods are not limited to the particular implementations described, and as such may vary. It is also to be understood that the terminology used herein is for the purpose of describing particular implementations only, and is not intended to be limiting. The scope of the present technology will be limited only by the appended claims.

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

As used herein, “about” means the recited quantity exactly and small variations within a limited range encompassing plus or minus 10% of the recited quantity. In other words, the limited range encompassed can include ±10%, ±9%, ±8%, ±7%, ±6%, ±5%, ±4%, ±3%, ±2%, ±1%, ±0.5%, ±0.2%, ±0.1%, ±0.05%, or smaller, as well as the recited value itself. Thus, by way of example, “about 10” should be understood to mean “10” and a range no larger than “9-11”.

As used herein, the term “bioproduction” is intended to mean production of a compound (e.g., bakuchiol, farnesene, farnesol, geosmin, geraniol, terpineol, limonene, myrcene, linalool, hinokitiol, pinene, cafestol, kahweol, cembrene, taxadiene, α-bisabolol, α-guaiene, bergamontene, and valencene) by way of biological or enzymatic synthesis (as opposed to chemical synthesis). In some implementations, bioproduction may be performed by a transgenic organism or microbe that has been engineered to express enzymes involved in the biological synthesis of a compound of interest (e.g., bakuchiol, farnesene, farnesol, geosmin, geraniol, terpineol, limonene, myrcene, linalool, hinokitiol, pinene, cafestol, kahweol, cembrene, taxadiene, α-bisabolol, α-guaiene, bergamontene, and valencene).

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Examples and implementations defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, the term “protein” is a biological macromolecule comprised of one or more chain(s) of amino acids. An “enzyme” is a type of protein that possesses a biological catalytic activity that accelerates chemical reaction. Thus, for the purposes of this disclosure, enzymes are an example of a protein that can catalyze a reaction, such as the production of bakuchiol from GPP/DMAPP/IPP and p-coumaric acid.

The terms “engineered cell” or “engineered host cell” refer to a modified cell wherein the modification can be selected from e.g., increased expression of a gene, inhibited expression of a gene, knockout of a gene, introduction of new gene(s), introduction of mutant gene(s), or mutation/genetic alteration of gene(s), wherein the increased expression or inhibited expression of a gene can be achieved by using common techniques in the art, such as gene deletion, changed gene copy number, changed gene promoter (e.g. by using a strong or weak promoter), etc. An engineered cell or engineered host cell may also include a cell that has been isolated. In some implementations, an engineered cell or engineered host cell is a transgenic cell. In some implementations, an engineered cell or engineered host cell is a transgenic cell capable of producing high levels of a compound or biomolecule of interest. An example of a host cell herein may be a microbial cell (e.g., bacteria, yeast, fungi, etc.).

The term “engineered microbial cell” refers to microbial cells that have been modified by the methods of the present disclosure. Thus, the terms include a microbial cell that has been genetically altered, modified, or engineered, such that it exhibits an altered, modified, or different genotype and/or phenotype (e.g., when the genetic modification affects coding nucleic acid sequences of the host cell), relative to a naturally-occurring organism from which it is derived. It is understood that in some implementations, the terms refer not only to the particular recombinant cell in question, but also to the progeny or potential progeny of such a cell.

For the purposes of this disclosure, all of the proteins, enzymes, and cells disclosed herein can be isolated in a form in which the protein is substantially free of other proteins, contaminants, or macromolecules (e.g., nucleic acids, lipids, etc.). However, it should be understood that an “isolated” protein or enzyme may not be 100% free of other proteins, contaminants, or macromolecules, and absolute purity is not required in order for a protein or enzyme to be considered “isolated.” It should also be understood that an “isolated” protein, enzyme, or cell can also be “engineered,” “non-engineered,” or “wild-type.” For the purposes of the present disclosure, an “engineered” protein, enzyme, or cell has been modified in some way (e.g., a substitution, addition, or deletion to an amino acid sequence in the case of a protein or enzyme; or heterologous expression of a non-native protein in the case of a cell) by the hand of man. A “non-engineered” protein, enzyme, or cell may refer to wild-types and naturally occurring irregularities, and a “wild type” is the phenotype or sequence of the typical form of a cell, protein, or enzyme as it occurs in nature (i.e., the “normal” or “standard” cell or protein sequence, as opposed to an engineered variant or a naturally occurring mutant).

As used herein, a “variant” when used in the context of referring to a protein means a protein sequence that is derived from a “parent” or reference sequence by incorporating one or more amino acid changes, which can include substitutions, deletions, or insertions. For the purposes of this disclosure, a variant may comprise an amino acid sequence that shares about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or up to about 100% sequence identity or homology with a reference or “parent” sequence. For purposes of this disclosure, the terms “variant” and “derivative” when used in the context of referring to a protein are used interchangeably.

As used herein, the term “misfolding” or “misfolded” when used in reference to a protein or enzyme means a protein conformational error has occurred. When a protein or enzyme misfolds, it may be non-functional, subject to aggregation, or both.

As used herein, “optional” or “optionally” means that the subsequently described event or circumstance may or may not occur, and that the description includes instances where said event or circumstance occurs and instances where it does not.

For the purpose of the description, a phrase in the form “A/B” or in the form “A and/or B” means (A), (B), or (A and B).

### II. Engineered Chaperone Proteins and Microbial Cells Comprising the Same

Heterologous expression of proteins (e.g., enzymes) can result in misfolding or deviations from the natural folding pattern. In many instances, proper protein folding may be needed for maintaining protein function, particularly in the case of enzymes. Accordingly, interventions that promote proper protein folding may be beneficial to producing enzymes with activity in heterologous expression systems, such as bacteria and yeast cells.

Chaperone proteins or molecular chaperones are proteins that assist the conformational folding or unfolding of large proteins or macromolecular protein complexes. There are a number of classes of molecular chaperones, all of which function to assist large proteins in proper protein folding during or after synthesis, and after partial denaturation. Chaperones may also be involved in the translocation of proteins.

The first molecular chaperones discovered are a type of assembly chaperones which assist in the assembly of nucleosomes from folded histones and DNA. One function of molecular chaperones is to prevent the aggregation of misfolded proteins, thus many chaperone proteins are classified as heat shock proteins, as the tendency for protein aggregation is increased by heat stress.

In some implementations, the majority of molecular chaperones do not convey any steric information for protein folding, and instead assist in protein folding by binding to and stabilizing folding intermediates until the polypeptide chain is fully translated. The specific mode of function of chaperones differs based on their target proteins and location. Various approaches have been applied to study the structure, dynamics and functioning of chaperones. Bulk biochemical measurements have informed us on the protein folding efficiency, and prevention of aggregation when chaperones are present during protein folding. Recent advances in single-molecule analysis have brought insights into structural heterogeneity of chaperones, folding intermediates and affinity of chaperones for unstructured and structured protein chains.

Some chaperone systems work as foldases: they support the folding of proteins in an ATP-dependent manner (for example, the GroEL/GroES or the DnaK/DnaJ/GrpE system). Although most newly synthesized proteins can fold in absence of chaperones, some may require them for proper folding. Other chaperones work as holdases: they bind folding intermediates to prevent their aggregation, for example DnaJ or Hsp33. Chaperones can also work as disaggregases, which interact with aberrant protein assemblies and revert them to monomers. Some chaperones can assist in protein degradation, leading proteins to protease systems, such as the ubiquitin-proteasome system in eukaryotes. Chaperone proteins participate in the folding of over half of all mammalian proteins.

### A. Engineered Chaperone Proteins

The present disclosure provides variants of wild-type Arabidopsis thaliana BIP1 (AtBIP1) with increased activity. In some implementations, the variant chaperones disclosed herein may have an amino acid sequence that has at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identity to wild-type AtBIP1. In some implementations, the variant chaperones disclosed herein may share at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology with wild-type AtBIP1. An amino acid sequence of wild-type AtBIP1 is set forth in SEQ ID NO: 52.

MARSFGANSTVVLAIIFFGCLFALSSAIEEATKLGSVIGIDLGTTYSCVGVYKNGHVEIIA NDQGNRITPSWVGFTDSERLIGEAAKNQAAVNPERTVFDVKRLIGRKFEDKEVQKDRKL VPYQIVNKDGKPYIQVKIKDGETKVFSPEEISAMILTKMKETAEAYLGKKIKDAVVTVPA YFNDAQRQATKDAGVIAGLNVARIINEPTAAAIAYGLDKKGGEKNILVFDLGGGTFDVS VLTIDNGVFEVLSTNGDTHLGGEDFDHRVMEYFIKLIKKKHQKDISKDNKALGKLRREC ERAKRALSSQHQVRVEIESLFDGVDFSEPLTRARFEELNNDLFRKTMGPVKKAMDDAG LQKSQIDEIVLVGGSTRIPKVQQLLKDFFEGKEPNKGVNPDEAVAYGAAVQGGILSGEG GDETKDILLLDVAPLTLGIETVGGVMTKLIPRNTVIPTKKSQVFTTYQDQQTTVSIQVFEG ERSLTKDCRLLGKFDLNGIPPAPRGTPQIEVTFEVDANGILNVKAEDKASGKSEKITITNE KGRLSQEEIDRMVKEAEEFAEEDKKVKEKIDARNALETYVYNMKNQVNDKDKLADKL EGDEKEKIEAATKEALEWLDENQNSEKEEYDEKLKEVEAVCNPIITAVYQRSGGAPGGA GGESSTEEEDESHDEL (SEQ ID NO: 52). Once the mature protein is expressed in vivo, the N-terminal methionine residue may be cleaved.

In some implementations, the variant chaperones disclosed herein may have an amino acid sequence that has about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identity to SEQ ID NO: 52. In some implementations, the variant chaperones disclosed herein may share about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homology with SEQ ID NO: 52.

In some implementations, the variant chaperones disclosed herein comprise an N-terminal deletion of from 1 to about 50 amino acid residues of SEQ ID NO: 52. For example, in some implementations, a variant comprises an N-terminal deletion of amino acid residues 1-5, 1-10, 1-15, 1-20, 1-25, 1-26, 1-27, 1-30, 1-35, 1-40, 1-45, or 1-50 of SEQ ID NO: 52. Thus, the disclosed variants may comprise an N-terminal deletion of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 consecutive amino acids.

In some implementations, the variant chaperones disclosed herein comprise an N-terminal deletion of 27 amino acid residues of SEQ ID NO: 52. For example, a chaperone protein variant can have an amino acid sequence comprising, or consisting of, SEQ ID NO: 53. Such a variant is referred to herein as “truncated AtBIP1” or “tAtBIP1.”

MIEEATKLGSVIGIDLGTTYSCVGVYKNGHVEIIANDQGNRITPSWVGFTDSERLIGEAA KNQAAVNPERTVFDVKRLIGRKFEDKEVQKDRKLVPYQIVNKDGKPYIQVKIKDGETK VFSPEEISAMILTKMKETAEAYLGKKIKDAVVTVPAYFNDAQRQATKDAGVIAGLNVA RIINEPTAAAIAYGLDKKGGEKNILVFDLGGGTFDVSVLTIDNGVFEVLSTNGDTHLGGE DFDHRVMEYFIKLIKKKHQKDISKDNKALGKLRRECERAKRALSSQHQVRVEIESLFDG VDFSEPLTRARFEELNNDLFRKTMGPVKKAMDDAGLQKSQIDEIVLVGGSTRIPKVQQL LKDFFEGKEPNKGVNPDEAVAYGAAVQGGILSGEGGDETKDILLLDVAPLTLGIETVGG VMTKLIPRNTVIPTKKSQVFTTYQDQQTTVSIQVFEGERSLTKDCRLLGKFDLNGIPPAPR GTPQIEVTFEVDANGILNVKAEDKASGKSEKITITNEKGRLSQEEIDRMVKEAEEFAEED KKVKEKIDARNALETYVYNMKNQVNDKDKLADKLEGDEKEKIEAATKEALEWLDENQ NSEKEEYDEKLKEVEAVCNPIITAVYQRSGGAPGGAGGESSTEEEDESHDEL (SEQ ID NO: 53). Once the mature protein is expressed in vivo, the N-terminal methionine residue may be cleaved.

In some implementations, a chaperone protein variant as described herein has at least about 80%—e.g., at least about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity to an N-terminal deletion variant thereof having a deletion of up to 27 N-terminal amino acid residues of SEQ ID NO: 52.

In some implementations, a chaperone protein variant as described consists of or comprises the amino acid sequence SEQ ID NO: 53. In some implementations, a chaperone protein variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 53.

In some implementations, a chaperone protein variant as described herein exhibits increased protein folding activity relative to wild-type AtBIP1 (SEQ ID NO: 52), such that its activity is at least about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, about 500%, about 550%, about 600%, about 650%, about 700%, about 750%, about 800%, about 850%, about 900%, about 950%, about 1000%, about 1100%, about 1200%, about 1300%, about 1400%, about 1500%, about 1600%, about 1700%, about 1800%, about 1900%, about 2000%, about 2250%, about 2500%, about 2750%, about 3000%, about 3250%, about 3500%, about 3750%, about 4000%, about 4250%, about 4500%, about 4750%, about 5000%, about 10000%, about 100000%, about 1000000%, about 10000000%, or about 20000000% or more than that of wild-type AtBIP1, as determined by an assay, such as one used to measure protein folding, protein translation, or another readout.

In some implementations, a variant as described herein exhibits increased protein folding activity relative to wild-type AtBIP1 (SEQ ID NO: 52), such that its activity is at least about 2-fold, about 4-fold, about 5-fold, about 10-fold, about 18-fold, about 20-fold, about 50-fold, about 100-fold, about 200-fold, about 1000-fold, about 5000-fold, about 10000-fold, about 20000-fold, about 50000-fold, about 100000-fold, about 200000-fold, about 500000-fold, or about 1000000-fold or more than that of wild-type AtBIP1, as determined by an assay, such as one used to measure protein folding, protein translation, or another readout.

### B. Engineered Microbial Cells Expressing Heterologous Chaperone Proteins

Provided herein are engineered microbial cells (e.g., bacteria or yeast cells) that express heterologous or engineered chaperone proteins. Generally, the heterologous or engineered chaperones will be co-expressed with one or more additional heterologous proteins.

In some implementations, an engineered microbial cell expresses a heterologous chaperone protein or variant thereof. The heterologous chaperone protein can be a chaperone protein that is not native to the microbial cell. For example, if the microbial cell is a Saccharomyces cerevisiae (S. cerevisiae) cell, the heterologous protein can be a chaperone protein or variant thereof that is not native to S. cerevisiae.

In some implementations, the heterologous chaperone protein is selected from the group consisting of AtBIP1 (SEQ ID NO: 52), tAtBIP1 (SEQ ID NO: 53), SSA4 (SEQ ID NO: 54), and KAR2 (SEQ ID NO: 55).

The amino acid sequence of SSA4 is: MSKAVGIDLGTTYSCVAHFANDRVEIIANDQGNRTTPSYVAFTDTERLIGDAAKNQAA MNPHNTVFDAKRLIGRKFDDPEVTNDAKHYPFKVIDKGGKPVVQVEYKGETKTFTPEEI SSMILTKMKETAENFLGTEVKDAVVTVPAYFNDSQRQATKDAGTIAGLNVLRIINEPTA AAIAYGLDKKSQKEHNVLIFDLGGGTFDVSLLSIDEGVFEVKATAGDTHLGGEDFDSRL VNFLAEEFKRKNKKDLTTNQRSLRRLRTAAERAKRTLSSSAQTSIEIDSLFEGIDFYTSITR ARFEELCADLFRSTLEPVEKVLADSKLDKSQIDEIVLVGGSTRIPKVQKLVSDFFNGKEP NRSINPDEAVAYGAAVQAAILTGDQSSTTQDLLLLDVAPLSLGIETAGGIMTKLIPRNSTI PTKKSEVFSTYADNQPGVLIQVFEGERTRTKDNNLLGKFELSGIPPAPRGVPQIEVTFDID ANGILNVSAVEKGTGKSNKITITNDKGRLSKEDIDKMVAEAEKFKAEDEQEAQRVQAK NQLESYAFTLKNSVSENNFKEKVGEEDAKKLEAAAQDAINWLDASQAASTEEYKERQK ELEGVANPIMSKFYGAAGGAPGAGPVPGAGAGPTGAPDNGPTVEEVD (SEQ ID NO: 54). Once the mature protein is expressed in vivo, the N-terminal methionine residue may be cleaved.

The amino acid sequence of KAR2 is: MFFNRLSAGKLLVPLSVVLYALFVVILPLQNSFHSSNVLVRGADDVENYGTVIGIDLGTT YSCVAVMKNGKTEILANEQGNRITPSYVAFTDDERLIGDAAKNQVAANPQNTIFDIKRLI GLKYNDRSVQKDIKIALPFNVVNKDGKPAVEVSVKGEKKVFTPEEISGMILGKMKQIAED YLGTKVTHAVVTVPAYFNDAQRQATKDAGTIAGLNVLRIVNEPTAAAIAYGLDKSDKE HQIIVYDLGGGTFDVSLLSIENGVFEVQATSGDTHLGGEDFDYKIVRQLIKAFKKKHGID VSDNNKALAKLKREAEKAKRALSSQMSTRIEIDSFVDODLSETLTRAKFEELNLDLFKK TLKPVEKVLQDSGLEKKDVDDIVLVGGSTRIPKVQQLLESYFDGKKASKGINPDEAVAY GAAVQAGVLSGEEGVEDIVLLDVNALTLGIETTGGVMTPLIKRNTAIPTKKSQIFSTAVD NQPTVMIKVYEGERAMSKDNNLLGKFELTGIPPAPRGVPQIEVTFALDANGILKVSATD KGTGKSESITITNDKGRLTQEEIDRMVEEAEKFASEDASIKAKVESRNKLENYAHSLKNQ VNGDLGEKLEEEDKETLLDAANDVLEWLDDNFETAIAEDFDEKFESLSKVAYPITSKLY GGADGSGAADYDDEDEDDDGDYFEHDEL (SEQ ID NO: 55). Once the mature protein is expressed in vivo, the N-terminal methionine residue may be cleaved.

The present disclosure provides an engineered microbial cell expressing a heterologous chaperone protein or variant thereof, wherein the chaperone protein or variant thereof comprises an amino acid sequence that has least 65% sequence identity to SEQ ID NO: 52, SEQ ID NO: 53, SEQ ID NO: 54, or SEQ ID NO: 55. In some implementations, the chaperone protein or variant thereof comprises an amino acid sequence that has at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 52, SEQ ID NO: 53, SEQ ID NO: 54, or SEQ ID NO: 55. In some implementations, the chaperone protein or variant thereof comprises, or consists, of SEQ ID NO: 52. In some implementations, the chaperone protein or variant thereof comprises, or consists, of SEQ ID NO: 53. In some implementations, the chaperone protein or variant thereof comprises, or consists, of SEQ ID NO: 54. In some implementations, the chaperone protein or variant thereof comprises, or consists, of SEQ ID NO: 55.

An engineered microbial cell can be of any strain or specific of microbial cell. An engineered microbial cell can be a eukaryotic cell or a prokaryotic cell. Non-limiting examples of microbial cells include cells of prokaryotic species such as Escherichia coli (E. coli), an Acinetobacter species, a Pseudomonas species, a Streptomyces species, and a Mycobacterium species, Klebsiella, Lactococcus, Mannheimia, Corynebacterium, Vibrio, and Bacillis. Non-limiting examples of microbial cells include cells of eukaryotic species such as Saccharomyces cerevisiae (S. cerevisiae) or other yeast species; a filamentous fungi, optionally selected from an Aspergillus species and a Trichoderma species; an algae, optionally selected from Botryococcus braunii, Chlorella sp., Crypthecodinium cohnii, Cylindrotheca sp., Nitzschia sp., Phaeodactylum tricornutum, Schizochytrium sp., and Tetraselmis suecia; and an amoeba, which is optionally Dictyostelium discoideum. Additional suitable eukaryotic cells include, but are not limited to, Pichia pastoris, Yarrowia hpolytica, Kluyveromyces marxianus, Rhodosporidium toruloides. Aspergillus (oryzae, nidulans, niger), Trichoderma reesei, and Penicillium chrysogenum.

In some implementations, an engineered microbial cell of the present disclosure expresses a heterologous chaperone protein or variant thereof, and further expresses a second heterologous protein. The second heterologous protein can be any heterologous protein. The second heterologous protein can be an enzyme, such as an engineered enzyme.

In some implementations, the second heterologous protein can be an enzyme that catalyzes production of bakuchiol, exhibits prenyltransferase activity, or both. For example, the second heterologous protein can be a bakuchiol-producing enzyme, such as any bakuchiol-producing enzyme described herein. In some implementations, an engineered microbial cell of the present disclosure expresses a heterologous chaperone protein and an enzyme that catalyzes production of bakuchiol, exhibits prenyltransferase activity, or both. In some implementations, an engineered microbial cell of the present disclosure expresses a heterologous chaperone protein, and at least one bakuchiol-producing enzyme. For examples, the second heterologous protein may comprise any one of SEQ ID NOs: 1-51.

In some implementations, the second heterologous protein can be an enzyme involved in the production of an isoprenoid, such as a sesquiterpene, a monoterpene, a diterpene, or a meroterpene. In some implementations, the second heterologous protein can be an enzyme involved in the production of farnesene, farnesol, geosmin, geraniol, terpineol, limonene, myrcene, linalool, hinokitiol, pinene, cafestol, kahweol, cembrene, taxadiene, α-bisabolol, α-guaiene, bergamontene, and valencene. Any of these enzymes described herein may be an engineered enzyme.

In some implementations, the engineer microbial cell may comprise a third, a fourth, or a fifth heterologous protein, any or all of which may be enzymes such as those discussed above.

### C. Methods of Expressing a Heterologous Protein in a Microbial Cell

The present disclosure provides methods of expressing a heterologous protein in a microbial cell, comprising co-expressing the heterologous protein and a heterologous chaperone protein.

In some implementations of a method of expressing a heterologous protein in a microbial cell, the microbial cell can be a eukaryotic cell or a prokaryotic cell. Non-limiting examples of microbial cells include cells of prokaryotic species such as Escherichia coli (E. coli), an Acinetobacter species, a Pseudomonas species, a Streptomyces species, and a Mycobacterium species, Klebsiella, Lactococcus, Mannheimia, Corynebacterium, Vibrio, and Bacillis. Non-limiting examples of microbial cells include cells of eukaryotic species such as Saccharomyces cerevisiae (S. cerevisiae) or other yeast species; a filamentous fungi, optionally selected from an Aspergillus species and a Trichoderma species; an algae, optionally selected from Botryococcus braunii, Chlorella sp., Crypthecodinium cohnii, Cylindrotheca sp., Nitzschia sp., Phaeodactylum tricornutum, Schizochytrium sp., and Tetraselmis suecia; and an amoeba, which is optionally Dictyostehum discoideum. Additional suitable eukaryotic cells include, but are not limited to, Pichia pastoris, Yarrowia lipolytica, Kluyveromyces marxianus, Rhodosporidium toruloides. Aspergillus (oryzae, nidulans, niger), Trichoderma reesei, and Penicillium chrysogenum.

In some implementations, a heterologous chaperone protein is a chaperone protein from Arabidopsis thaliana. In some implementations, the heterologous chaperone protein is Arabidopsis thaliana BIP1 (AtBIP1; SEQ ID NO: 52) or a variant thereof. In some implementations, the heterologous chaperone protein is tAtBIP1 (SEQ ID NO: 53). In some implementations, the heterologous chaperone protein comprises an amino acid sequence that has at least 65%—e.g., at least 70%, at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO: 52 or SEQ ID NO: 53. In some implementations, the heterologous chaperone protein comprises, or consists of, SEQ ID NO: 52. In some implementations, the heterologous chaperone protein is a variant of SEQ ID NO: 52 comprising an N-terminal deletion of 1-50 (i.e., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) amino acids from SEQ ID NO: 52. In some implementations, the heterologous chaperone protein comprises, or consists of, SEQ ID NO: 53. In some implementations, the heterologous chaperone protein comprises an amino acid sequence that has at least 65%—e.g., at least 70%, at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO: 54 or SEQ ID NO: 55. In some implementations, the heterologous chaperone protein comprises, or consists of, SEQ ID NO: 54. In some implementations, the heterologous chaperone protein comprises, or consists of, SEQ ID NO: 55.

In a method of expressing a heterologous protein in a microbial cell in accordance with the present disclosure, the heterologous protein can be an enzyme. In some implementations, the heterologous protein is an enzyme that catalyzes production of bakuchiol, exhibits prenyltransferase activity, or both. In some implementations, the heterologous protein is a HMGR. In some implementations, the heterologous protein is an enzyme involved in the production of an isoprenoid, such as a sesquiterpene, a monoterpene, a diterpene, or a meroterpene. In some implementations, the heterologous protein can be an enzyme involved in the production of farnesene, farnesol, geosmin, geraniol, terpineol, limonene, myrcene, linalool, hinokitiol, pinene, cafestol, kahweol, cembrene, taxadiene, α-bisabolol, α-guaiene, bergamontene, and valencene.

In some implementations, expression of the heterologous protein is increased relative to expression of the heterologous protein in a microbial cell that does not co-express the heterologous chaperone protein.

### D. Methods of Enhancing the Folding of a Heterologous Protein

The present disclosure provides methods of enhancing the folding of a heterologous protein in a microbial cell by enhancing folding of the heterologous protein in the microbial cell, comprising co-expressing the heterologous protein with a heterologous chaperone protein.

In some implementations, a method of facilitating folding of a heterologous protein in a microbial cell comprises co-expressing in the microbial cell the heterologous protein and a heterologous chaperone protein or variant thereof, wherein the heterologous chaperone protein comprises an amino acid sequence that has at least 70%—e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO: 52, SEQ ID NO: 53, SEQ ID NO: 54, or SEQ ID NO: 55.

In some implementations of a method of facilitating folding of a heterologous protein in a microbial cell, a microbial cell can be a eukaryotic cell or a prokaryotic cell. Non-limiting examples of microbial cells include cells of prokaryotic species such as Escherichia coli (E. coli), an Acinetobacter species, a Pseudomonas species, a Streptomyces species, and a Mycobacterium species, Klebsiella, Lactococcus, Mannheimia, Corynebacterium, Vibrio, and Bacillis. Non-limiting examples of microbial cells include cells of eukaryotic species such as Saccharomyces cerevisiae (S. cerevisiae) or other yeast species; a filamentous fungi, optionally selected from an Aspergillus species and a Trichoderma species; an algae, optionally selected from Botryococcus braunii, Chlorella sp., Crypthecodinium cohnii, Cylindrotheca sp., Nitzschia sp., Phaeodactylum tricornutum, Schizochytrium sp., and Tetraselmis suecia; and an amoeba, which is optionally Dictyostelium discoideum. Additional suitable eukaryotic cells include, but are not limited to, Pichia pastoris, Yarrowia hpolytica, Kluyveromyces marxianus, Rhodosporidium toruloides. Aspergillus (oryzae, nidulans, niger), Trichoderma reesei, and Penicillium chrysogenum.

In some implementations, expression of the heterologous protein is enhanced relative to expression of the heterologous protein in a microbial cell that does not co-express the heterologous chaperone protein. In some implementations, the heterologous protein misfolds when expressed in a microbial cell that does not co-express the heterologous chaperone protein.

In some implementations of a method of facilitating folding of a heterologous protein in a microbial cell, a heterologous chaperone protein or variant thereof co-expressed together with the heterologous protein comprises, or consists, of SEQ ID NO: 52. In some implementations, the heterologous chaperone protein or variant thereof is a variant of SEQ ID NO: 52 comprising an N-terminal deletion of 1-50 (i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) amino acids from SEQ ID NO: 52. In some implementations, the heterologous chaperone protein or variant thereof comprises, or consists of, SEQ ID NO: 53. In some implementations, the heterologous chaperone protein or variant thereof comprises, or consists of, SEQ ID NO: 54. In some implementations, the heterologous chaperone protein or variant thereof comprises, or consists of, SEQ ID NO: 55.

In a method of facilitating the folding of a heterologous protein in a microbial cell in accordance with the present disclosure, the heterologous protein can be an enzyme. In some implementations, the heterologous protein is or comprises an enzyme that catalyzes production of bakuchiol, exhibits prenyltransferase activity, or both. In some implementations, expression of the heterologous protein is enhanced relative to expression of the heterologous protein in a microbial cell that does not co-express the heterologous chaperone protein.

### III. Bakuchiol

Bakuchiol is a phenolic compound having a single hydroxyl group on the aromatic ring and an unsaturated hydrocarbon chain. It has been engineered from the seeds of Psoralea. corylifolia. The chemical structure of bakuchiol is provided below

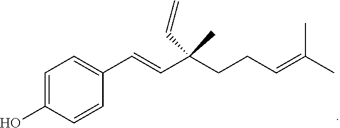 [SMILES: C=C[C@@](C)(/C=C/c1ccc(O)cc1)CCC=C(C)C]

In one implementation, the bakuchiol chemical structure is also presented as

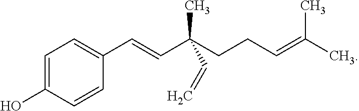 [SMILES: C=C[C@](C)(/C=C/c1ccc(O)cc1)CCC=C(C)C]

Bakuchiol has been reported as possessing antibacterial activity, anti-inflammatory activity, anti-cancer activity, anti-oxidant activity, and other beneficial properties. As a result, it may be used in supplements, cosmetics, and other consumer products, and it may be employed for pharmaceutical use. However, there are currently a number of limitations associated with the use of this compound due primarily to its low concentration in natural sources, as well as the presence of co-existing toxic components. One of the main problems related to the use of bakuchiol compositions engineered from plants in the Psoralea genus is the presence of psoralens, such as psoralen and isopsoralen, which are associated with a number of health risks. Additionally, pre-existing methods of chemically synthesizing bakuchiol or extracting it from plants are generally inefficient and resource intensive.

The presently disclosed proteins and methods make it possible to bioproduce bakuchiol, thus addressing the limitations of pre-existing chemical and extraction-based methodologies.

### IV. Bakuchiol-Producing Proteins and Nucleic Acids

In one implementation of the methods described herein is based on bakuchiol being produced by a prenyltransferase enzyme through a mechanism involving geranyl pyrophosphate (GPP), dimethylallyl pyrophosphate (DMAPP), isopentenyl pyrophosphate (IPP), or a combination thereof, and p-coumaric acid. Two enzymes capable of producing bakuchiol when expressed in S. cerevisiae comprise amino acid sequences as set forth below:

(SEQ ID NO: 1; referred to herein as “BAK28”)
MHEYANMRHRQHNLKHNYGGIEGVSTCEDWARNFVVNAASGESLESHEAQ
HHTPETLWGSIKQFCDAFYRFSRPHVIIGTAVNIIVMSSLALEKSSDISP
KFFIGLFQVIVTILSMNIYTAGINQLTDIEIDKINKPYLPLASGEYSYKT
GVTIITLCAILSLGVGWIVGSPPLFWSNFAYFVLGTVYSIDLPLMRWKSH
PALAALFFFVIRGLTFHVGFFLHLQTHVFKRPMMIPKPVMFGTAFMSFFY
VIIAFFKDIPDIEGDKDHGVKSLTMRLGQKRVFWICVSLLLTGYGAAIVV
GATSSFLWCKLITVSGHALLASIFWNRAKSVDLKSHQEITSLYMFMWKLF
YAEYFIIPLMR,
and
(SEQ ID NO: 2; referred to herein as “BAK36”)
MASMFLGSLPLASSVNYIGRITRSKNCTESYHATSYITNASSNKTEKIKH
EYANMRHRQHNLKHNYGGIEGVSTCEDWARNFVVNAASGESLESHEAQHH
TPETLWGSIKQFCDAFYRFSRPHVIIGTAVNIIVMSSLALEKSSDISPKF
FIGLFQVIVTILSMNIYTAGINQLTDIEIDKINKPYLPLASGEYSYKTGV
TIITLCAILSLGVGWIVGSPPLFWSNFAYFVLGTVYSIDLPLMRWKSHPA
LAALFFFVIRGLTFHVGFFLHLQTHVFKRPMMIPKSVMFGTAFMSFFYVI
IAFFKDIPDIEGDKDHGVKSLTMRLGQERVFWICVSLLLTGYGAAIVVGA
TSSFLWCKLITVSGHALLASIFWNRAKSVDLKSHQEITSLYMFMWKLFYA
EYFIIPLMR.

When these enzymes are expressed in vivo, the N-terminal methionine residue may be cleaved to form a mature enzyme.

The present disclosure further provides additional example putative prenyltranferase enzymes capable of converting p-coumaric acid and GPP/DMAPP/IPP into bakuchiol. Thus, the present disclosure provides bakuchiol-producing enzymes that have at least about 65%—e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 1. Similarly, the present disclosure provides bakuchiol-producing enzymes that have at least about 65%—e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 2. In some implementations, the bakuchiol-producing enzyme may share at least about 90% identity with SEQ ID NO: 1 or SEQ ID NO: 2. In some implementations, the bakuchiol-producing enzyme may share at least about 95% identity with SEQ ID NO: 1 or SEQ ID NO: 2. In some implementations, the bakuchiol-producing enzyme may share at least about 99% identity with SEQ ID NO: 1 or SEQ ID NO: 2. Thus, this disclosure encompasses enzymes with varying degrees of sequence identity compared to SEQ ID NOs: 1 and 2, so long as the protein exhibits prenyltranferase activity, is able to produce bakuchiol, or both.

SEQ ID NO: 1 and SEQ ID NO: 2 are structurally similar and share similar amino acid sequences. SEQ ID NO: 1 is missing 49 amino acid residues at its N terminus that are present in SEQ ID NO: 2. Aside from this truncation, there are only two other amino acid substitutions across the length of the protein sequences. This indicates that SEQ ID NOs: 1 and 2 may represent splice variants of the same gene, and further shows in one implementation that the minimum domain involved for activity may be less than the entire 409 amino acid sequence of SEQ ID NO: 2, and a protein that is longer than the 361 amino acid sequence of SEQ ID NO: 1 may be active as well. Accordingly, the present disclosure encompasses protein sequences that are the same length, longer, or shorter than SEQ ID NO: 1 or SEQ ID NO: 2.

For example, a bakuchiol-producing enzyme of the present disclosure may comprise SEQ ID NO: 1 (i.e., it is 361 amino acids or longer). In some implementations, a bakuchiol-producing enzyme of the present disclosure may comprise 361 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 1. In some implementations, a bakuchiol-producing enzyme of the present disclosure may consist of SEQ ID NO: 1. In some implementations, a bakuchiol-producing enzyme of the present disclosure may consist of 361 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 1. In some implementations, a bakuchiol-producing enzyme of the present disclosure may comprise about 365, about 370, about 375, about 380, about 385, about 390, about 395, about 400, about 405, about 410, about 415, about 420, about 425, about 430, about 435, about 440, or about 450 amino acids, wherein at least about 361 amino acids of the enzyme have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 1. In some implementations, a bakuchiol-producing enzyme of the present disclosure may comprise about 365 to about 700 amino acids—e.g., about 365 to about 650 amino acids, about 365 to about 600 amino acids, about 365 to about 550 amino acids, about 365 to about 500 amino acids, about 365 to about 450 amino acids, about 365 to about 400 amino acids, about 375 to about 700 amino acids, about 375 to about 650 amino acids, about 375 to about 600 amino acids, about 375 to about 550 amino acids, about 375 to about 500 amino acids, about 375 to about 450 amino acids, about 375 to about 400 amino acids, about 385 to about 700 amino acids, about 385 to about 650 amino acids, about 385 to about 600 amino acids, about 385 to about 550 amino acids, about 385 to about 500 amino acids, about 385 to about 450 amino acids, about 385 to about 400 amino acids, about 395 to about 700 amino acids, about 395 to about 650 amino acids, about 395 to about 600 amino acids, about 395 to about 550 amino acids, about 395 to about 500 amino acids, about 395 to about 450 amino acids, or about 395 to about 400 amino acids, or any values in between; wherein at least about 361 amino acids of the enzyme have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 1.

Similarly, a bakuchiol-producing enzyme of the present disclosure may comprise SEQ ID NO: 2 (i.e., it is 409 amino acids or longer). In some implementations, a bakuchiol-producing enzyme of the present disclosure may comprise 409 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 2. In some implementations, a bakuchiol-producing enzyme of the present disclosure may consist of SEQ ID NO: 2. In some implementations, a bakuchiol-producing enzyme of the present disclosure may consist of 409 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 2. In some implementations, a bakuchiol-producing enzyme of the present disclosure may comprise about 410, about 415, about 420, about 425, about 430, about 435, about 440, or about 450 amino acids, wherein at least about 409 amino acids of the enzyme have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 2. In some implementations, a bakuchiol-producing enzyme of the present disclosure may comprise about 410 to about 700 amino acids- e.g., about 410 to about 650 amino acids, about 410 to about 600 amino acids, about 410 to about 550 amino acids, about 410 to about 500 amino acids, about 410 to about 450 amino acids, about 420 to about 700 amino acids, about 420 to about 650 amino acids, about 420 to about 600 amino acids, about 420 to about 550 amino acids, about 420 to about 500 amino acids, about 420 to about 450 amino acids, about 430 to about 700 amino acids, about 430 to about 650 amino acids, about 430 to about 600 amino acids, about 430 to about 550 amino acids, about 430 to about 500 amino acids, about 430 to about 450 amino acids, about 440 to about 700 amino acids, about 440 to about 650 amino acids, about 440 to about 600 amino acids, about 440 to about 550 amino acids, about 440 to about 500 amino acids, or about 440 to about 450 amino acids, or any values in between, wherein at least about 409 amino acids of the enzyme have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 2.

Bakuchiol-producing enzymes described herein include also those that are shorter than SEQ ID NO: 1 or SEQ ID NO: 2. A bakuchiol-producing enzyme may be less than 409 or less than 361 amino acids in length, so long as the enzyme has a catalytic domain that has at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 1 or SEQ ID NO: 2. In some implementations at least about 50, at least about 75, at least about 100, at least about 125, at least about 150, at least about 175, at least about 200, at least about 225, at least about 250, at least about 275, at least about 300, at least about 325, or at least about 350 amino acids of the enzyme can have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 1 or SEQ ID NO: 2, so long as the enzyme exhibits prenyltransferase activity, is able to catalyze bakuchiol production, or both.

Indeed, for the purposes of this disclosure, any of the disclosed proteins is considered a “bakuchiol-producing protein” or a “bakuchiol-producing enzyme” if the protein exhibits prenyltransferase activity, catalyzes the production of bakuchiol, or both. Further, it should be understood for the purposes of this disclosure that a protein “exhibits” prenyltransferase activity or “catalyzes” the production of bakuchiol if the foregoing functions are significant enough to be measured, observed, or detected using conventional methods in the art (e.g., mass spectrometry).

The present disclosure also provides nucleic acids comprising a nucleic acid sequence encoding any one of the proteins disclosed herein. A nucleic acid sequence can be designed/determined based on a known amino acid sequence as a result of known codon specificity. Thus, in some implementations, the nucleic acid may comprise a nucleic acid sequence encoding SEQ ID NO: 1, SEQ ID NO: 2, or a protein that has at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 1 or SEQ ID NO: 2, so long as the protein exhibits prenyltransferase activity, is able to catalyze bakuchiol production, or both.

Because the disclosed proteins may be of particular value when used in the context of transgenic expression in a microbial chassis, the nucleic acid sequence encoding any one of the disclosed proteins may be codon-optimized for a given expression system. For example, the nucleic acid sequence may be codon-optimized for expression is a yeast system, such as S. cerevisiae. Alternatively, the nucleic acid sequence may be codon-optimized for expression is a prokaryotic system, such as E. coli.

Nucleic acids that encode a bakuchiol-producing protein can be incorporated into an expression vector or expression cassette. The nucleic acid can be transduced or transformed into a transgenic cell such that the nucleic acid sequence encoding the bakuchiol-producing protein is integrated into the genome of the host cell or transgenic cell. Alternatively, the nucleic acid sequence encoding the bakuchiol-producing protein may be expressed without integration into the host genome (e.g., in the form of a plasmid). For those implementations in which genome integration is desired, any suitable methods of integration can be used, including but not limited to Cas-based systems (e.g., Cas9, Cas12, etc.), homologous recombination, gene gun, conjugation protocols, lambda red, etc.

An expression cassette or vector for expressing the nucleic acid sequence encoding the bakuchiol-producing protein may comprise a promoter and a terminator. Any suitable promoters may be used, including but not limited to GAL1, TEF2, TEF1, TDH3, ENO2, CCW12, EF-1a promoter, CMV immediate early, HSV thymidine kinase, early and late SV40, LTRs from retrovirus, and mouse metallothionein-I. In some implementations, an inducible or repressible promoter, such as GAL1, GAL2, GAL7, GAL10, CUP1, MET3, MET17, or MET25, may be used. Inducible promoters operably link the expression of a target gene (e.g., the nucleic acid sequence encoding a bakuchiol-producing protein) to a specific signal or a particular biotic or abiotic factor. Types of inducible promoters that may be utilized in the disclosed may include, but are not limited to, chemically-inducible promoters (i.e., antibiotics, steroids, metals, etc.), light-inducible promoters, heat-inducible promoters, and hypoxia-inducible promoters. Transcription terminators that may be used are also known in the art (see Bittner et al., Methods in Enzymol. 153: 516-544 (1987)), and include but are not limited to GAT2, Rho-dependent terminators, Rho-independent terminators, poly-A sequences, and the like (see Curran et al., Metab. Eng., 19: 88-97 (2013)).

For the purposes of the present disclosure, any of the foregoing proteins can be expressed in a host cell or transgenic cell and any of the foregoing nucleic acids may incorporated into a host cell or transgenic cell in order to produce bakuchiol according to the disclosed methods.

### V. Bakuchiol-Producing Enzyme Variants

Described herein are bakuchiol-producing enzyme variants comprising an amino acid sequence that is a variant of the amino acid sequence of the bakuchiol-producing enzymes set forth in SEQ ID NO: 1 or SEQ ID NO: 2. These enzyme variants may also be referred to an “engineered bakuchiol-producing enzymes,” as the variants comprise at least one change relative to a naturally occurring sequence. Thus, for the purposes of the present disclosure, a variant sequence has at least one substitution, addition, or deletion, relative to SEQ ID NO: 1 or SEQ ID NO: 2. In some implementations, the at least one substitution, addition, or deletion increases the production of bakuchiol by the variant relative to the wild-type bakuchiol-producing enzyme. The disclosed substitutions and deletions may be combined to produce synergistic effects on bakuchiol production.

The present disclosure provides variants of BAK28 and BAK36 with improved bakuchiol-producing activity. In some implementations, a variant may have an amino acid has about 80%, about 85%, about 90%, about 91%, about 92%, about 93%, about 94%, about 95%, about 96%, about 97%, about 98%, or about 99% identity to wild-type BAK28 (SEQ ID NO: 1) or BAK36 (SEQ ID NO: 2). In some implementations, a variant may share about 80%, about 85%, about 90%, about 91%, about 92%, about 93%, about 94%, about 95%, about 96%, about 97%, about 98%, or about 99% homology to wild-type BAK28 (SEQ ID NO: 1) or BAK36 (SEQ ID NO: 2). For instance, in some implementations, a variant may comprise the amino acids residues conserved between BAK28 and BAK36.

It is observed that, in many instances, an N-terminal deletion, surprisingly, improves bakuchiol-producing activity of both BAK28 and BAK36. This observation suggests that the C-terminus of BAK28 and BAK36 may be involved in catalyzing the production of bakuchiol, while the N-terminus may play a lesser or no role in the production of bakuchiol. For example, the bakuchiol-producing enzyme variant may comprise an N-terminal deletion of from 1 to 100 amino acid residues of SEQ ID NO: 1, or an N-terminal deletion of from 1 to 150 amino acid residues of SEQ ID NO: 2. In some implementations, a variant may comprise an N-terminal deletion of amino acid residues 1-20, 1-21, 1-22, 1-23, 1-24, 1-25, 1-26, 1-27, 1-28, 1-29, 1-30, 1-31, 1-32, 1-33, 1-34, 1-35, 1-36, 1-37, 1-38, 1-39, 1-40, 1-41, 1-42, 1-43, 1-44, 1-45, 1-46, 1-47, 1-48, 1-49, 1-50, 1-51, 1-52, 1-53, 1-54, 1-55, 1-56, 1-57, 1-58, 1-59, 1-60, 1-61, 1-62, 1-63, 1-64, 1-65, 1-66, 1-67, 1-68, 1-69, 1-70, 1-71, or 1-72, of SEQ ID NO: 1. In some implementations, a variant may comprise an N-terminal deletion of amino acid residues 2-20, 2-21, 2-22, 2-23, 2-24, 2-25, 2-26, 2-27, 2-28, 2-29, 2-30, 2-31, 2-32, 2-33, 2-34, 2-35, 2-36, 2-37, 2-38, 2-39, 2-40, 2-41, 2-42, 2-43, 2-44, 2-45, 2-46, 2-47, 2-48, 2-49, 2-50, 2-51, 2-52, 2-53, 2-54, 2-55, 2-56, 2-57, 2-58, 2-59, 2-60, 2-61, 2-62, 2-63, 2-64, 2-65, 2-66, 2-67, 2-68, 2-69, 2-70, 2-71, or 2-72, of SEQ ID NO: 1. In some implementations, a variant comprises an N-terminal deletion of amino acid residues 1-20, 1-21, 1-22, 1-23, 1-24, 1-25, 1-26, 1-27, 1-28, 1-29, 1-30, 1-31, 1-32, 1-33, 1-34, 1-35, 1-36, 1-37, 1-38, 1-39, 1-40, 1-41, 1-42, 1-43, 1-44, 1-45, 1-46, 1-47, 1-48, 1-49, 1-50, 1-51, 1-52, 1-53, 1-54, 1-55, 1-56, 1-57, 1-58, 1-59, 1-60, 1-61, 1-62, 1-63, 1-64, 1-65, 1-66, 1-67, 1-68, 1-69, 1-70, 1-71, -172, 1-73, 1-74, 1-75, 1-76, 1-78, 1-79, 1-80, 1-81, 1-82, 1-83, 1-84, 1-85, 1-86, 1-87, 1-88, 1-89, 1-90, 1-91, 1-92, 1-93, 1-94, 1-95, 1-96, 1-97, 1-98, 1-99, 1-100, 1-101, 1-102, 1-103, 1-104, 1-105, 1-106, 1-107, 1-108, 1-109, 1-110, 1-111, 1-112, 1-113, 1-114, 1-115, 1-116, 1-117, 1-118, 1-119, or 1-120 of SEQ ID NO: 2.

Thus, a bakuchiol-producing enzyme variant may comprise an N-terminal deletion of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52. 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 consecutive amino acids from SEQ ID NO: 1 or SEQ ID NO: 2. In other words, the present disclosure provides, an engineered bakuchiol-producing enzyme comprising an N-terminal deletion of 1 to about 120 amino acids (e.g., 2 to about 120 amino acids) from the N-terminus of the enzyme, wherein the enzyme catalyzes production of bakuchiol, exhibits prenyltransferase activity, or both. The N-terminal deletion, in several instances, surprisingly is found to increase catalyzation of production of bakuchiol, prenyltransferase activity, or both, relative to a non-engineered enzyme comprising the same amino acid sequence but without the N-terminal deletion.

In some implementations, a variant may additionally or alternatively comprise a deletion at the C-terminus of the protein. Such a C-terminal deletion may encompass 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more consecutive amino acids. However, in some implementations, a variant does not comprise any deletions to its C-terminal domain. Indeed, in some implementations, deletions from the C-terminus or modifications to the amino acid sequence of the C-terminus may be detrimental to bakuchiol-producing activity.

The amino acid sequence of BAK36(T1), a variant comprising an N-terminal deletion of the T1 region of BAK36, and further example variants thereof are set forth in Table 1 below.

TABLE 1
Amino Acid Sequences of N-terminally truncated BAK36 and
Exemplary Variants

| | SEQ | |
|---|---|---|
| | ID | |
| Enzyme | NO. | Sequence |
| BAK36(T1) | 3 | MTNASSNKTEKIKHEYANMRHRQHNLKHNYGGIEGVSTCE |
| | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRWKSHP |
| | | ALAALFFFVIRGLTFHVGFFLHLQTHVFKRPMMIPKSVMFGT |
| | | AFMSFFYVIIAFFKDIPDIEGDKDHGVKSLTMRLGQERVFWIC |
| | | VSLLLTGYGAAIVVGATSSFLWCKLITVSGHALLASIFWNRA |
| | | KSVDLKSHQEITSLYMFMWKLFYAEYFIIPLMR |
| BAK36(T1) | 4 | MTNASSNKTEKIKHEYANMRHRQHNLKHNYGGIEGVSTCE |
| E54F | | DWARNFVVNAASGFSLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRWKSHP |
| | | ALAALFFFVIRGLTFHVGFFLHLQTHVFKRPMMIPKSVMFGT |
| | | AFMSFFYVIIAFFKDIPDIEGDKDHGVKSLTMRLGQERVFWIC |
| | | VSLLLTGYGAAIVVGATSSFLWCKLITVSGHALLASIFWNRA |
| | | KSVDLKSHQEITSLYMFMWKLFYAEYFIIPLMR |
| BAK36(T1) | 5 | MTNASSNKTEKIKHEYANMRHRQHNLKHNYGGIEGVSTCE |
| G71D | | DWARNFVVNAASGESLESHEAQHHTPETLWDSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRWKSHP |
| | | ALAALFFFVIRGLTFHVGFFLHLQTHVFKRPMMIPKSVMFGT |
| | | AFMSFFYVIIAFFKDIPDIEGDKDHGVKSLTMRLGQERVFWIC |
| | | VSLLLTGYGAAIVVGATSSFLWCKLITVSGHALLASIFWNRA |
| | | KSVDLKSHQEITSLYMFMWKLFYAEYFIIPLMR |
| BAK36(T1) | 6 | MTNASSNKTEKIKHEYANMRHRQHNLKHNYGGIEGVSTCE |
| S108L | | DWARNFVVNAASGESLESHEAQHHTPETLWGSIKQFCDAFY |
| | | RFSRPHVIIGTAVNIIVMSSLALEKSLDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRWKSHP |
| | | ALAALFFFVIRGLTFHVGFFLHLQTHVFKRPMMIPKSVMFGT |
| | | AFMSFFYVIIAFFKDIPDIEGDKDHGVKSLTMRLGQERVFWIC |
| | | VSLLLTGYGAAIVVGATSSFLWCKLITVSGHALLASIFWNRA |
| | | KSVDLKSHQEITSLYMFMWKLFYAEYFIIPLMR |
| BAK36(T1) | 7 | MTNASSNKTEKIKHEYANMRHRQHNLKHNYGGIEGVSTCE |
| T162H | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKHGVTIITLCA |
| | | ILSLGVGWIVGSPPLFWSNFAYFVLGTVYSIDLPLMRWKSHP |
| | | ALAALFFFVIRGLTFHVGFFLHLQTHVFKRPMMIPKSVMFGT |
| | | AFMSFFYVIIAFFKDIPDIEGDKDHGVKSLTMRLGQERVFWIC |
| | | VSLLLTGYGAAIVVGATSSFLWCKLITVSGHALLASIFWNRA |
| | | KSVDLKSHQEITSLYMFMWKLFYAEYFIIPLMR |
| BAK36(T1) | 8 | MTNASSNKTEKIKHEYANMRHRQHNLKHNYGGIEGVSTCE |
| P185V | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPVLFWSNFAYFVLGTVYSIDLPLMRWKSHP |
| | | ALAALFFFVIRGLTFHVGFFLHLQTHVFKRPMMIPKSVMFGT |
| | | AFMSFFYVIIAFFKDIPDIEGDKDHGVKSLTMRLGQERVFWIC |
| | | VSLLLTGYGAAIVVGATSSFLWCKLITVSGHALLASIFWNRA |
| | | KSVDLKSHQEITSLYMFMWKLFYAEYFIIPLMR |
| BAK36(T1) | 9 | MTNASSNKTEKIKHEYANMRHRQHNLKHNYGGIEGVSTCE |
| V199G | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTGYSIDLPLMRWKSHP |
| | | ALAALFFFVIRGLTFHVGFFLHLQTHVFKRPMMIPKSVMFGT |
| | | AFMSFFYVIIAFFKDIPDIEGDKDHGVKSLTMRLGQERVFWIC |
| | | VSLLLTGYGAAIVVGATSSFLWCKLITVSGHALLASIFWNRA |
| | | KSVDLKSHQEITSLYMFMWKLFYAEYFIIPLMR |
| BAK36(T1) | 10 | MTNASSNKTEKIKHEYANMRHRQHNLKHNYGGIEGVSTCE |
| P205L | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLLLMRWKSHP |
| | | ALAALFFFVIRGLTFHVGFFLHLQTHVFKRPMMIPKSVMFGT |
| | | AFMSFFYVIIAFFKDIPDIEGDKDHGVKSLTMRLGQERVFWIC |
| | | VSLLLTGYGAAIVVGATSSFLWCKLITVSGHALLASIFWNRA |
| | | KSVDLKSHQEITSLYMFMWKLFYAEYFIIPLMR |
| BAK36(T1) | 11 | MTNASSNKTEKIKHEYANMRHRQHNLKHNYGGIEGVSTCE |
| P205V | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLVLMRWKSHP |
| | | ALAALFFFVIRGLTFHVGFFLHLQTHVFKRPMMIPKSVMFGT |
| | | AFMSFFYVIIAFFKDIPDIEGDKDHGVKSLTMRLGQERVFWIC |
| | | VSLLLTGYGAAIVVGATSSFLWCKLITVSGHALLASIFWNRA |
| | | KSVDLKSHQEITSLYMFMWKLFYAEYFIIPLMR |
| BAK36(T1) | 12 | MTNASSNKTEKIKHEYANMRHRQHNLKHNYGGIEGVSTCE |
| L206Y | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPYMRWKSHP |
| | | ALAALFFFVIRGLTFHVGFFLHLQTHVFKRPMMIPKSVMFGT |
| | | AFMSFFYVIIAFFKDIPDIEGDKDHGVKSLTMRLGQERVFWIC |
| | | VSLLLTGYGAAIVVGATSSFLWCKLITVSGHALLASIFWNRA |
| | | KSVDLKSHQEITSLYMFMWKLFYAEYFIIPLMR |
| BAK36(T1) | 13 | MTNASSNKTEKIKHEYANMRHRQHNLKHNYGGIEGVSTCE |
| W209S | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRSKSHPA |
| | | LAALFFFVIRGLTFHVGFFLHLQTHVFKRPMMIPKSVMFGTA |
| | | FMSFFYVIIAFFKDIPDIEGDKDHGVKSLTMRLGQERVFWICV |
| | | SLLLTGYGAAIVVGATSSFLWCKLITVSGHALLASIFWNRAK |
| | | SVDLKSHQEITSLYMFMWKLFYAEYFIIPLMR |
| BAK36(T1) | 14 | MTNASSNKTEKIKHEYANMRHRQHNLKHNYGGIEGVSTCE |
| W209C | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRCKSHP |
| | | ALAALFFFVIRGLTFHVGFFLHLQTHVFKRPMMIPKSVMFGT |
| | | AFMSFFYVIIAFFKDIPDIEGDKDHGVKSLTMRLGQERVFWIC |
| | | VSLLLTGYGAAIVVGATSSFLWCKLITVSGHALLASIFWNRA |
| | | KSVDLKSHQEITSLYMFMWKLFYAEYFIIPLMR |
| BAK36(T1) | 15 | MTNASSNKTEKIKHEYANMRHRQHNLKHNYGGIEGVSTCE |
| W209V | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRVKSHP |
| | | ALAALFFFVIRGLTFHVGFFLHLQTHVFKRPMMIPKSVMFGT |
| | | AFMSFFYVIIAFFKDIPDIEGDKDHGVKSLTMRLGQERVFWIC |
| | | VSLLLTGYGAAIVVGATSSFLWCKLITVSGHALLASIFWNRA |
| | | KSVDLKSHQEITSLYMFMWKLFYAEYFIIPLMR |
| BAK36(T1) | 16 | MTNASSNKTEKIKHEYANMRHRQHNLKHNYGGIEGVSTCE |
| W209T | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRTKSHP |
| | | ALAALFFFVIRGLTFHVGFFLHLQTHVFKRPMMIPKSVMFGT |
| | | AFMSFFYVIIAFFKDIPDIEGDKDHGVKSLTMRLGQERVFWIC |
| | | VSLLLTGYGAAIVVGATSSFLWCKLITVSGHALLASIFWNRA |
| | | KSVDLKSHQEITSLYMFMWKLFYAEYFIIPLMR |
| BAK36(T1) | 17 | MTNASSNKTEKIKHEYANMRHRQHNLKHNYGGIEGVSTCE |
| W209Y | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRYKSHP |
| | | ALAALFFFVIRGLTFHVGFFLHLQTHVFKRPMMIPKSVMFGT |
| | | AFMSFFYVIIAFFKDIPDIEGDKDHGVKSLTMRLGQERVFWIC |
| | | VSLLLTGYGAAIVVGATSSFLWCKLITVSGHALLASIFWNRA |
| | | KSVDLKSHQEITSLYMFMWKLFYAEYFIIPLMR |
| BAK36(T1) | 18 | MTNASSNKTEKIKHEYANMRHRQHNLKHNYGGIEGVSTCE |
| W209R | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRRKSHP |
| | | ALAALFFFVIRGLTFHVGFFLHLQTHVFKRPMMIPKSVMFGT |
| | | AFMSFFYVIIAFFKDIPDIEGDKDHGVKSLTMRLGQERVFWIC |
| | | VSLLLTGYGAAIVVGATSSFLWCKLITVSGHALLASIFWNRA |
| | | KSVDLKSHQEITSLYMFMWKLFYAEYFIIPLMR |
| BAK36(T1) | 19 | MTNASSNKTEKIKHEYANMRHRQHNLKHNYGGIEGVSTCE |
| W209M | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRMKSHP |
| | | ALAALFFFVIRGLTFHVGFFLHLQTHVFKRPMMIPKSVMFGT |
| | | AFMSFFYVIIAFFKDIPDIEGDKDHGVKSLTMRLGQERVFWIC |
| | | VSLLLTGYGAAIVVGATSSFLWCKLITVSGHALLASIFWNRA |
| | | KSVDLKSHQEITSLYMFMWKLFYAEYFIIPLMR |
| BAK36(T1) | 20 | MTNASSNKTEKIKHEYANMRHRQHNLKHNYGGIEGVSTCE |
| W209Q | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRQKSHP |
| | | ALAALFFFVIRGLTFHVGFFLHLQTHVFKRPMMIPKSVMFGT |
| | | AFMSFFYVIIAFFKDIPDIEGDKDHGVKSLTMRLGQERVFWIC |
| | | VSLLLTGYGAAIVVGATSSFLWCKLITVSGHALLASIFWNRA |
| | | KSVDLKSHQEITSLYMFMWKLFYAEYFIIPLMR |
| BAK36(T1) | 21 | MTNASSNKTEKIKHEYANMRHRQHNLKHNYGGIEGVSTCE |
| L226M | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRWKSHP |
| | | ALAALFFFVIRGMTFHVGFFLHLQTHVFKRPMMIPKSVMFG |
| | | TAFMSFFYVIIAFFKDIPDIEGDKDHGVKSLTMRLGQERVFWI |
| | | CVSLLLTGYGAAIVVGATSSFLWCKLITVSGHALLASIFWNR |
| | | AKSVDLKSHQEITSLYMFMWKLFYAEYFIIPLMR |
| BAK36(T1) | 22 | MTNASSNKTEKIKHEYANMRHRQHNLKHNYGGIEGVSTCE |
| L234Q | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRWKSHP |
| | | ALAALFFFVIRGLTFHVGFFQHLQTHVFKRPMMIPKSVMFGT |
| | | AFMSFFYVIIAFFKDIPDIEGDKDHGVKSLTMRLGQERVFWIC |
| | | VSLLLTGYGAAIVVGATSSFLWCKLITVSGHALLASIFWNRA |
| | | KSVDLKSHQEITSLYMFMWKLFYAEYFIIPLMR |
| BAK36(T1) | 23 | MTNASSNKTEKIKHEYANMRHRQHNLKHNYGGIEGVSTCE |
| F257E | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRWKSHP |
| | | ALAALFFFVIRGLTFHVGFFLHLQTHVFKRPMMIPKSVMFGT |
| | | AEMSFFYVIIAFFKDIPDIEGDKDHGVKSLTMRLGQERVFWI |
| | | CVSLLLTGYGAAIVVGATSSFLWCKLITVSGHALLASIFWNR |
| | | AKSVDLKSHQEITSLYMFMWKLFYAEYFIIPLMR |
| BAK36(T1) | 24 | MTNASSNKTEKIKHEYANMRHRQHNLKHNYGGIEGVSTCE |
| K269R | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRWKSHP |
| | | ALAALFFFVIRGLTFHVGFFLHLQTHVFKRPMMIPKSVMFGT |
| | | AFMSFFYVIIAFFRDIPDIEGDKDHGVKSLTMRLGQERVFWIC |
| | | VSLLLTGYGAAIVVGATSSFLWCKLITVSGHALLASIFWNRA |
| | | KSVDLKSHQEITSLYMFMWKLFYAEYFIIPLMR |
| BAK36(T1) | 25 | MTNASSNKTEKIKHEYANMRHRQHNLKHNYGGIEGVSTCE |
| I274L | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRWKSHP |
| | | ALAALFFFVIRGLTFHVGFFLHLQTHVFKRPMMIPKSVMFGT |
| | | AFMSFFYVIIAFFKDIPDLEGDKDHGVKSLTMRLGQERVFWI |
| | | CVSLLLTGYGAAIVVGATSSFLWCKLITVSGHALLASIFWNR |
| | | AKSVDLKSHQEITSLYMFMWKLFYAEYFIIPLMR |
| BAK36(T1) | 26 | MTNASSNKTEKIKHEYANMRHRQHNLKHNYGGIEGVSTCE |
| D279C | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRWKSHP |
| | | ALAALFFFVIRGLTFHVGFFLHLQTHVFKRPMMIPKSVMFGT |
| | | AFMSFFYVIIAFFKDIPDIEGDKCHGVKSLTMRLGQERVFWIC |
| | | VSLLLTGYGAAIVVGATSSFLWCKLITVSGHALLASIFWNRA |
| | | KSVDLKSHQEITSLYMFMWKLFYAEYFIIPLMR |
| BAK36(T1) | 27 | MTNASSNKTEKIKHEYANMRHRQHNLKHNYGGIEGVSTCE |
| D279K | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRWKSHP |
| | | ALAALFFFVIRGLTFHVGFFLHLQTHVFKRPMMIPKSVMFGT |
| | | AFMSFFYVIIAFFKDIPDIEGDKKHGVKSLTMRLGQERVFWIC |
| | | VSLLLTGYGAAIVVGATSSFLWCKLITVSGHALLASIFWNRA |
| | | KSVDLKSHQEITSLYMFMWKLFYAEYFIIPLMR |
| BAK36(T1) | 28 | MTNASSNKTEKIKHEYANMRHRQHNLKHNYGGIEGVSTCE |
| D279R | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRWKSHP |
| | | ALAALFFFVIRGLTFHVGFFLHLQTHVFKRPMMIPKSVMFGT |
| | | AFMSFFYVIIAFFKDIPDIEGDKRHGVKSLTMRLGQERVFWIC |
| | | VSLLLTGYGAAIVVGATSSFLWCKLITVSGHALLASIFWNRA |
| | | KSVDLKSHQEITSLYMFMWKLFYAEYFIIPLMR |
| BAK36(T1) | 29 | MTNASSNKTEKIKHEYANMRHRQHNLKHNYGGIEGVSTCE |
| D279M | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRWKSHP |
| | | ALAALFFFVIRGLTFHVGFFLHLQTHVFKRPMMIPKSVMFGT |
| | | AFMSFFYVIIAFFKDIPDIEGDKMHGVKSLTMRLGQERVFWI |
| | | CVSLLLTGYGAAIVVGATSSFLWCKLITVSGHALLASIFWNR |
| | | AKSVDLKSHQEITSLYMFMWKLFYAEYFIIPLMR |
| BAK36(T1) | 30 | MTNASSNKTEKIKHEYANMRHRQHNLKHNYGGIEGVSTCE |
| D279L | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRWKSHP |
| | | ALAALFFFVIRGLTFHVGFFLHLQTHVFKRPMMIPKSVMFGT |
| | | AFMSFFYVIIAFFKDIPDIEGDKLHGVKSLTMRLGQERVFWIC |
| | | VSLLLTGYGAAIVVGATSSFLWCKLITVSGHALLASIFWNRA |
| | | KSVDLKSHQEITSLYMFMWKLFYAEYFIIPLMR |
| BAK36(T1) | 31 | MTNASSNKTEKIKHEYANMRHRQHNLKHNYGGIEGVSTCE |
| M287V | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRWKSHP |
| | | ALAALFFFVIRGLTFHVGFFLHLQTHVFKRPMMIPKSVMFGT |
| | | AFMSFFYVIIAFFKDIPDIEGDKDHGVKSLTVRLGQERVFWIC |
| | | VSLLLTGYGAAIVVGATSSFLWCKLITVSGHALLASIFWNRA |
| | | KSVDLKSHQEITSLYMFMWKLFYAEYFIIPLMR |
| BAK36(T1) | 32 | MTNASSNKTEKIKHEYANMRHRQHNLKHNYGGIEGVSTCE |
| M287F | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRWKSHP |
| | | ALAALFFFVIRGLTFHVGFFLHLQTHVFKRPMMIPKSVMFGT |
| | | AFMSFFYVIIAFFKDIPDIEGDKDHGVKSLTFRLGQERVFWIC |
| | | VSLLLTGYGAAIVVGATSSFLWCKLITVSGHALLASIFWNRA |
| | | KSVDLKSHQEITSLYMFMWKLFYAEYFIIPLMR |
| BAK36(T1) | 33 | MTNASSNKTEKIKHEYANMRHRQHNLKHNYGGIEGVSTCE |
| M287Y | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRWKSHP |
| | | ALAALFFFVIRGLTFHVGFFLHLQTHVFKRPMMIPKSVMFGT |
| | | AFMSFFYVIIAFFKDIPDIEGDKDHGVKSLTYRLGQERVFWIC |
| | | VSLLLTGYGAAIVVGATSSFLWCKLITVSGHALLASIFWNRA |
| | | KSVDLKSHQEITSLYMFMWKLFYAEYFIIPLMR |
| BAK36(T1) | 34 | MTNASSNKTEKIKHEYANMRHRQHNLKHNYGGIEGVSTCE |
| I310V | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRWKSHP |
| | | ALAALFFFVIRGLTFHVGFFLHLQTHVFKRPMMIPKSVMFGT |
| | | AFMSFFYVIIAFFKDIPDIEGDKDHGVKSLTMRLGQERVFWIC |
| | | VSLLLTGYGAAVVVGATSSFLWCKLITVSGHALLASIFWNR |
| | | AKSVDLKSHQEITSLYMFMWKLFYAEYFIIPLMR |
| BAK36(T1) | 35 | MTNASSNKTEKIKHEYANMRHRQHNLKHNYGGIEGVSTCE |
| V312W | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRWKSHP |
| | | ALAALFFFVIRGLTFHVGFFLHLQTHVFKRPMMIPKSVMFGT |
| | | AFMSFFYVIIAFFKDIPDIEGDKDHGVKSLTMRLGQERVFWIC |
| | | VSLLLTGYGAAIVWGATSSFLWCKLITVSGHALLASIFWNRA |
| | | KSVDLKSHQEITSLYMFMWKLFYAEYFIIPLMR |
| BAK36(T1) | 36 | MTNASSNKTEKIKHEYANMRHRQHNLKHNYGGIEGVSTCE |
| V312A | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRWKSHP |
| | | ALAALFFFVIRGLTFHVGFFLHLQTHVFKRPMMIPKSVMFGT |
| | | AFMSFFYVIIAFFKDIPDIEGDKDHGVKSLTMRLGQERVFWIC |
| | | VSLLLTGYGAAIVAGATSSFLWCKLITVSGHALLASIFWNRA |
| | | KSVDLKSHQEITSLYMFMWKLFYAEYFIIPLMR |
| BAK36(T1) | 37 | MTNASSNKTEKIKHEYANMRHRQHNLKHNYGGIEGVSTCE |
| V312F | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRWKSHP |
| | | ALAALFFFVIRGLTFHVGFFLHLQTHVFKRPMMIPKSVMFGT |
| | | AFMSFFYVIIAFFKDIPDIEGDKDHGVKSLTMRLGQERVFWIC |
| | | VSLLLTGYGAAIVFGATSSFLWCKLITVSGHALLASIFWNRA |
| | | KSVDLKSHQEITSLYMFMWKLFYAEYFIIPLMR |
| BAK36(T1) | 38 | MTNASSNKTEKIKHEYANMRHRQHNLKHNYGGIEGVSTCE |
| V312G | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRWKSHP |
| | | ALAALFFFVIRGLTFHVGFFLHLQTHVFKRPMMIPKSVMFGT |
| | | AFMSFFYVIIAFFKDIPDIEGDKDHGVKSLTMRLGQERVFWIC |
| | | VSLLLTGYGAAIVGGATSSFLWCKLITVSGHALLASIFWNRA |
| | | KSVDLKSHQEITSLYMFMWKLFYAEYFIIPLMR |
| BAK36(T1) | 39 | MTNASSNKTEKIKHEYANMRHRQHNLKHNYGGIEGVSTCE |
| V312Y | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRWKSHP |
| | | ALAALFFFVIRGLTFHVGFFLHLQTHVFKRPMMIPKSVMFGT |
| | | AFMSFFYVIIAFFKDIPDIEGDKDHGVKSLTMRLGQERVFWIC |
| | | VSLLLTGYGAAIVYGATSSFLWCKLITVSGHALLASIFWNRA |
| | | KSVDLKSHQEITSLYMFMWKLFYAEYFIIPLMR |
| BAK36(T1) | 40 | MTNASSNKTEKIKHEYANMRHRQHNLKHNYGGIEGVSTCE |
| V312C | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRWKSHP |
| | | ALAALFFFVIRGLTFHVGFFLHLQTHVFKRPMMIPKSVMFGT |
| | | AFMSFFYVIIAFFKDIPDIEGDKDHGVKSLTMRLGQERVFWIC |
| | | VSLLLTGYGAAIVCGATSSFLWCKLITVSGHALLASIFWNRA |
| | | KSVDLKSHQEITSLYMFMWKLFYAEYFIIPLMR |
| BAK36(T1) | 41 | MTNASSNKTEKIKHEYANMRHRQHNLKHNYGGIEGVSTCE |
| V312L | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRWKSHP |
| | | ALAALFFFVIRGLTFHVGFFLHLQTHVFKRPMMIPKSVMFGT |
| | | AFMSFFYVIIAFFKDIPDIEGDKDHGVKSLTMRLGQERVFWIC |
| | | VSLLLTGYGAAIVLGATSSFLWCKLITVSGHALLASIFWNRA |
| | | KSVDLKSHQEITSLYMFMWKLFYAEYFIIPLMR |
| BAK36(T1) | 42 | MTNASSNKTEKIKHEYANMRHRQHNLKHNYGGIEGVSTCE |
| G313I | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRWKSHP |
| | | ALAALFFFVIRGLTFHVGFFLHLQTHVFKRPMMIPKSVMFGT |
| | | AFMSFFYVIIAFFKDIPDIEGDKDHGVKSLTMRLGQERVFWIC |
| | | VSLLLTGYGAAIVVIATSSFLWCKLITVSGHALLASIFWNRA |
| | | KSVDLKSHQEITSLYMFMWKLFYAEYFIIPLMR |
| BAK36(T1) | 43 | MTNASSNKTEKIKHEYANMRHRQHNLKHNYGGIEGVSTCE |
| S317P | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRWKSHP |
| | | ALAALFFFVIRGLTFHVGFFLHLQTHVFKRPMMIPKSVMFGT |
| | | AFMSFFYVIIAFFKDIPDIEGDKDHGVKSLTMRLGQERVFWIC |
| | | VSLLLTGYGAAIVVGATSPFLWCKLITVSGHALLASIFWNRA |
| | | KSVDLKSHQEITSLYMFMWKLFYAEYFIIPLMR |
| BAK36(T1) | 44 | MTNASSNKTEKIKHEYANMRHRQHNLKHNYGGIEGVSTCE |
| S317I | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRWKSHP |
| | | ALAALFFFVIRGLTFHVGFFLHLQTHVFKRPMMIPKSVMFGT |
| | | AFMSFFYVIIAFFKDIPDIEGDKDHGVKSLTMRLGQERVFWIC |
| | | VSLLLTGYGAAIVVGATSIFLWCKLITVSGHALLASIFWNRA |
| | | KSVDLKSHQEITSLYMFMWKLFYAEYFIIPLMR |
| BAK36(T1) | 45 | MTNASSNKTEKIKHEYANMRHRQHNLKHNYGGIEGVSTCE |
| F318R | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRWKSHP |
| | | ALAALFFFVIRGLTFHVGFFLHLQTHVFKRPMMIPKSVMFGT |
| | | AFMSFFYVIIAFFKDIPDIEGDKDHGVKSLTMRLGQERVFWIC |
| | | VSLLLTGYGAAIVVGATSSRLWCKLITVSGHALLASIFWNRA |
| | | KSVDLKSHQEITSLYMFMWKLFYAEYFIIPLMR |
| BAK36(T1) | 46 | MTNASSNKTEKIKHEYANMRHRQHNLKHNYGGIEGVSTCE |
| F318G | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRWKSHP |
| | | ALAALFFFVIRGLTFHVGFFLHLQTHVFKRPMMIPKSVMFGT |
| | | AFMSFFYVIIAFFKDIPDIEGDKDHGVKSLTMRLGQERVFWIC |
| | | VSLLLTGYGAAIVVGATSSGLWCKLITVSGHALLASIFWNRA |
| | | KSVDLKSHQEITSLYMFMWKLFYAEYFIIPLMR |
| BAK36(T1) | 47 | MTNASSNKTEKIKHEYANMRHRQHNLKHNYGGIEGVSTCE |
| L319P | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRWKSHP |
| | | ALAALFFFVIRGLTFHVGFFLHLQTHVFKRPMMIPKSVMFGT |
| | | AFMSFFYVIIAFFKDIPDIEGDKDHGVKSLTMRLGQERVFWIC |
| | | VSLLLTGYGAAIVVGATSSFPWCKLITVSGHALLASIFWNRA |
| | | KSVDLKSHQEITSLYMFMWKLFYAEYFIIPLMR |
| BAK36(T1) | 48 | MTNASSNKTEKIKHEYANMRHRQHNLKHNYGGIEGVSTCE |
| W320D | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRWKSHP |
| | | ALAALFFFVIRGLTFHVGFFLHLQTHVFKRPMMIPKSVMFGT |
| | | AFMSFFYVIIAFFKDIPDIEGDKDHGVKSLTMRLGQERVFWIC |
| | | VSLLLTGYGAAIVVGATSSFLDCKLITVSGHALLASIFWNRA |
| | | KSVDLKSHQEITSLYMFMWKLFYAEYFIIPLMR |
| BAK36(T1) | 49 | MTNASSNKTEKIKHEYANMRHRQHNLKHNYGGIEGVSTCE |
| T325G | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRWKSHP |
| | | ALAALFFFVIRGLTFHVGFFLHLQTHVFKRPMMIPKSVMFGT |
| | | AFMSFFYVIIAFFKDIPDIEGDKDHGVKSLTMRLGQERVFWIC |
| | | VSLLLTGYGAAIVVGATSSFLWCKLIGVSGHALLASIFWNRA |
| | | KSVDLKSHQEITSLYMFMWKLFYAEYFIIPLMR |
| BAK36(T1) | 50 | MTNASSNKTEKIKHEYANMRHRQHNLKHNYGGIEGVSTCE |
| S342G | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRWKSHP |
| | | ALAALFFFVIRGLTFHVGFFLHLQTHVFKRPMMIPKSVMFGT |
| | | AFMSFFYVIIAFFKDIPDIEGDKDHGVKSLTMRLGQERVFWIC |
| | | VSLLLTGYGAAIVVGATSSFLWCKLITVSGHALLASIFWNRA |
| | | KGVDLKSHQEITSLYMFMWKLFYAEYFIIPLMR |
| BAK36(T1) | 51 | MTNASSNKTEKIKHEYANMRHRQHNLKHNYGGIEGVSTCE |
| L354F | | DWARNFVVNAASGESLESHEAQHHTPETLWGSIKQFCDAFY |
| | | RFSRPHVIIGTAVNIIVMSSLALEKSSDISPKFFIGLFQVIVTILS |
| | | MNIYTAGINQLTDIEIDKINKPYLPLASGEYSYKTGVTIITLCA |
| | | ILSLGVGWIVGSPPLFWSNFAYFVLGTVYSIDLPLMRWKSHP |
| | | ALAALFFFVIRGLTFHVGFFLHLQTHVFKRPMMIPKSVMFGT |
| | | AFMSFFYVIIAFFKDIPDIEGDKDHGVKSLTMRLGQERVFWIC |
| | | VSLLLTGYGAAIVVGATSSFLWCKLITVSGHALLASIFWNRA |
| | | KSVDLKSHQEITSFYMFMWKLFYAEYFIIPLMR |

Additionally, the N-terminal methionine of any of the forgoing variants (i.e., SEQ ID NOs: 3-51) may also be cleaved off in a purified product or after expression in vivo. However, all amino acid position designations disclosed in this table take the methionine residue into account for the purpose of maintaining amino acid numbering conventions.

In some implementations, a bakuchiol-producing enzyme variant as described herein may have at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the wild-type BAK28 enzyme (SEQ ID NO: 1), or to an N-terminal deletion variant thereof having a deletion of up to 73 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52. 53, 54, 55, 56, 57, 58, 59, 60, 61, 62, 63, 64, 65, 66, 67, 68, 69, 70, 71, 72, or 73) N-terminal amino acid residues of SEQ ID NO: 1.

In some implementations, a bakuchiol-producing enzyme variant as described herein may have at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the wild-type BAK36 enzyme (SEQ ID NO: 2), or to an N-terminal deletion variant thereof having a deletion of up to 120 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52. 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120) N-terminal amino acid residues of SEQ ID NO: 2.

In some implementations, a bakuchiol-producing enzyme variant as described herein may comprise, or consist of, SEQ ID NO: 3. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 3. In some implementations, a bakuchiol-producing enzyme variant can comprise an amino acid sequence comprising at least one (e.g., 1, 2, 3, 4, or 5 or more) substitution mutation(s) relative to SEQ ID NO: 3 at one or more amino acid positions selected from 54, 71, 108, 162, 185, 199, 205, 206, 209, 226, 234, 257, 269, 274, 279, 287, 310, 312, 313, 317, 318, 319, 320, 325, 342, and 354.

In some implementations, a bakuchiol-producing enzyme variant as described herein may comprise, or consist, of SEQ ID NO: 4. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 4.

In some implementations, a bakuchiol-producing enzyme variant as described herein may comprise, or consist of, SEQ ID NO: 5. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 5.

In some implementations, a bakuchiol-producing enzyme variant as described herein may comprise, or consist, of SEQ ID NO: 6. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 6.

In some implementations, a bakuchiol-producing enzyme variant as described herein may comprise, or consist, of SEQ ID NO: 7. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 7.

In some implementations, a bakuchiol-producing enzyme variant as described herein may comprise, or consist, of SEQ ID NO: 8. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 8.

In some implementations, a bakuchiol-producing enzyme variant as described herein may comprise, or consist, of SEQ ID NO: 9. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 9.

In some implementations, a bakuchiol-producing enzyme variant as described herein may comprise, or consist, of SEQ ID NO: 10. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 10.

In some implementations, a bakuchiol-producing enzyme variant as described herein may comprise, or consist, of SEQ ID NO: 11. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 11.

In some implementations, a bakuchiol-producing enzyme variant as described herein may comprise, or consist, of SEQ ID NO: 12. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 12.

In some implementations, a bakuchiol-producing enzyme variant as described herein may comprise, or consist, of SEQ ID NO: 13. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 13.

In some implementations, a bakuchiol-producing enzyme variant as described herein may comprise, or consist, of SEQ ID NO: 14. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 14.

In some implementations, a bakuchiol-producing enzyme variant as described herein may comprise, or consist, of SEQ ID NO: 15. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 15.

In some implementations, a bakuchiol-producing enzyme variant as described herein may comprise, or consist, of SEQ ID NO: 16. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 16.

In some implementations, a bakuchiol-producing enzyme variant as described herein may comprise, or consist, of SEQ ID NO: 17. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 17.

In some implementations, a bakuchiol-producing enzyme variant as described herein may comprise, or consist, of SEQ ID NO: 18. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 18.

In some implementations, a bakuchiol-producing enzyme variant as described herein may comprise, or consist, of SEQ ID NO: 19. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 19.

In some implementations, a bakuchiol-producing enzyme variant as described herein may comprise, or consist, of SEQ ID NO: 20. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 20.

In some implementations, a bakuchiol-producing enzyme variant as described herein may comprise, or consist, of SEQ ID NO: 21. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 21.

In some implementations, a bakuchiol-producing enzyme variant as described herein may comprise, or consist, of SEQ ID NO: 22. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 22.

In some implementations, a bakuchiol-producing enzyme variant as described herein may comprise, or consist, of SEQ ID NO: 23. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 23.

In some implementations, a bakuchiol-producing enzyme variant as described herein may comprise, or consist, of SEQ ID NO: 24. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 24.

In some implementations, a bakuchiol-producing enzyme variant as described herein may comprise, or consist, of SEQ ID NO: 25. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 25.

In some implementations, a bakuchiol-producing enzyme variant as described herein may comprise, or consist, of SEQ ID NO: 26. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 26.

In some implementations, a bakuchiol-producing enzyme variant as described herein may comprise, or consist, of SEQ ID NO: 27. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 27.

In some implementations, a bakuchiol-producing enzyme variant as described herein may comprise, or consist, of SEQ ID NO: 28. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 28.

In some implementations, a bakuchiol-producing enzyme variant as described herein may comprise, or consist, of SEQ ID NO: 29. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 29.

In some implementations, a bakuchiol-producing enzyme variant as described herein may comprise, or consist, of SEQ ID NO: 30. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 30.

In some implementations, a bakuchiol-producing enzyme variant as described herein may comprise, or consist, of SEQ ID NO: 31. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 31.

In some implementations, a bakuchiol-producing enzyme variant as described herein may comprise, or consist, of SEQ ID NO: 32. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 32.

In some implementations, a bakuchiol-producing enzyme variant as described herein may comprise, or consist, of SEQ ID NO: 33. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 33.

In some implementations, a bakuchiol-producing enzyme variant as described herein may comprise, or consist, of SEQ ID NO: 34. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 34.

In some implementations, a bakuchiol-producing enzyme variant as described herein may comprise, or consist, of SEQ ID NO: 35. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 35.

In some implementations, a bakuchiol-producing enzyme variant as described herein may comprise, or consist, of SEQ ID NO: 36. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 36.

In some implementations, a bakuchiol-producing enzyme variant as described herein may comprise, or consist, of SEQ ID NO: 37. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 37.

In some implementations, a bakuchiol-producing enzyme variant as described herein may comprise, or consist, of SEQ ID NO: 38. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 38.

In some implementations, a bakuchiol-producing enzyme variant as described herein may comprise, or consist, of SEQ ID NO: 39. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 39.

In some implementations, a bakuchiol-producing enzyme variant as described herein may comprise, or consist, of SEQ ID NO: 40. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 40.

In some implementations, a bakuchiol-producing enzyme variant as described herein may comprise, or consist, of SEQ ID NO: 41. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 41.

In some implementations, a bakuchiol-producing enzyme variant as described herein may comprise, or consist, of SEQ ID NO: 42. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 42.

In some implementations, a bakuchiol-producing enzyme variant as described herein may comprise, or consist, of SEQ ID NO: 43. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 43.

In some implementations, a bakuchiol-producing enzyme variant as described herein may comprise, or consist, of SEQ ID NO: 44. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 44.

In some implementations, a bakuchiol-producing enzyme variant as described herein may comprise, or consist, of SEQ ID NO: 45. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 45.

In some implementations, a bakuchiol-producing enzyme variant as described herein may comprise, or consist, of SEQ ID NO: 46. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 46.

In some implementations, a bakuchiol-producing enzyme variant as described herein may comprise, or consist, of SEQ ID NO: 47. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 47.

In some implementations, a bakuchiol-producing enzyme variant as described herein may comprise, or consist, of SEQ ID NO: 48. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 48.

In some implementations, a bakuchiol-producing enzyme variant as described herein may comprise, or consist, of SEQ ID NO: 49. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 49.

In some implementations, a bakuchiol-producing enzyme variant as described herein may comprise, or consist, of SEQ ID NO: 50. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 50.

In some implementations, a bakuchiol-producing enzyme variant as described herein may comprise, or consist, of SEQ ID NO: 51. In some implementations, a bakuchiol-producing enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 51.

The present disclosure provides an engineered bakuchiol-producing enzyme comprising at least one amino acid substitution at position 54, 71, 108, 162, 185, 199, 205, 206, 209, 226, 234, 257, 269, 274, 279, 287, 310, 312, 313, 317, 318, 319, 320, 325, 342 or 354 of SEQ ID NO: 3. In some implementations, the engineered bakuchiol-producing enzyme may comprise 2, 3, 4, or 5 or more amino acid substitutions at residues selected from 54, 71, 108, 162, 185, 199, 205, 206, 209, 226, 234, 257, 269, 274, 279, 287, 310, 312, 313, 317, 318, 319, 320, 325, 342, and 354 of SEQ ID NO: 3. In some implementations, the enzyme may comprise at least one amino acid substitution (e.g., 1, 2, 3, 4, 5 or more) selected from the group consisting of E54F, G71D, S108L, T162H, P185V, V199G, P205L, P205V, L206Y, W209S, W209C, W209V, W209T, W209Y, W209R, W209M, W209Q, L226M, L234Q, F257E, K269R, I274L, D279C, D279K, D279R, D279M, D279L, M287V, M287F, M287Y, I310V, V312W, V312A, V312F, V312G, V312Y, V312C, V312L, G313I, S317P, S317I, F318R, F318G, L319P, W320D, T325G, S342G, and L354F, relative to SEQ ID NO: 3.

Similarly, the present disclosure provides an engineered enzyme comprising an amino acid sequence that is a variant of SEQ ID NO: 1, wherein the amino acid sequence comprises at least one substitution mutation relative to SEQ ID NO: 1 at one or more amino acid positions selected from 42, 59, 96, 150, 173, 187, 193, 194 197, 214, 222, 245, 257, 262, 267, 275, 298, 300, 301, 305, 306, 307, 308, 313, 330, and 342. In some implementations, the engineered bakuchiol-producing enzyme may comprise 2, 3, 4, or 5 or more amino acid substitutions at residues selected from 42, 59, 96, 150, 173, 187, 193, 194, 197, 214, 222, 245, 257, 262, 267, 275, 298, 300, 301, 305, 306, 307, 308, 313, 330, and 342. The present disclosure also provides an engineered enzyme comprising an amino acid sequence that is a variant of SEQ ID NO: 2, wherein the amino acid sequence comprises at least one substitution mutation relative to SEQ ID NO: 2 at one or more amino acid positions selected from 90, 107, 144, 198, 221, 235, 241, 242, 245, 262, 270, 293, 305, 310, 315, 323, 346, 348, 349, 353, 354, 355, 356, 361, 378, and 390. In some implementations, the engineered bakuchiol-producing enzyme may comprise 2, 3, 4, or 5 or more amino acid substitutions at residues selected from 90, 107, 144, 198, 221, 235, 241, 242, 245, 262, 270, 293, 305, 310, 315, 323, 346, 348, 349, 353, 354, 355, 356, 361, 378, and 390.

The present disclosure and data provided herein indicate that amino acid positions corresponding to residues 42, 59, 96, 150, 173, 187, 193, 194, 197, 214, 222, 245, 257, 262, 267, 275, 298, 300, 301, 305, 306, 307, 308, 313, 330, and 342 of SEQ ID NO: 1 and residues 90, 107, 144, 198, 221, 235, 241, 242, 245, 262, 270, 293, 305, 310, 315, 323, 346, 348, 349, 353, 354, 355, 356, 361, 378, and 390 of SEQ ID NO: 2 are involved in enzymatic function and that substitutions at these residues tend to increase bakuchiol production. Accordingly, the same increase in activity is expected to be observed in other bakuchiol-producing enzymes with shared homology. Accordingly, the present disclosure provides an engineered enzyme that catalyzes production of bakuchiol, exhibits prenyltransferase activity, or both, wherein the engineered enzyme comprises at least one substitution mutation selected from:
(a) substitution of a glutamate (E) corresponding to the E at position 42 of SEQ ID NO: 1 or position 90 of SEQ ID NO: 2
(b) substitution of a glycine (G) corresponding to the G at position 59 of SEQ ID NO: 1 or position 107 of SEQ ID NO: 2;
(c) substitution of a serine (S) corresponding to the S at position 96 of SEQ ID NO: 1 or position 144 of SEQ ID NO: 2;
(d) substitution of threonine (T) corresponding to the T at position 150 of SEQ ID NO: 1 or position 198 of SEQ ID NO: 2;
(e) substitution of proline (P) corresponding to the P at position 173 of SEQ ID NO: 1 or position 221 of SEQ ID NO: 2;
(f) substitution of valine (V) corresponding to the V at position 187 of SEQ ID NO: 1 or position 235of SEQ ID NO: 2;
(g) substitution of proline (P) corresponding to the P at position 193 of SEQ ID NO: 1 or position 241of SEQ ID NO: 2;
(h) substitution of leucine (L) corresponding to the L at position 194 of SEQ ID NO: 1 or position 242 of SEQ ID NO: 2;
(i) substitution of tryptophan (W) corresponding to the W at position 197 of SEQ ID NO: 1 or position 245 of SEQ ID NO: 2;
(j) substitution of leucine (L) corresponding to the L at position 214 of SEQ ID NO: 1 or position 262 of SEQ ID NO: 2;
(k) substitution of leucine (L) corresponding to the L at position 222 of SEQ ID NO: 1 or position 270 of SEQ ID NO: 2;
(l) substitution of phenylalanine (F) corresponding to the F at position 245 of SEQ ID NO: 1 or position 293 of SEQ ID NO: 2;
(m) substitution of lysine (K) corresponding to the K at position 257 of SEQ ID NO: 1 or position 305 of SEQ ID NO: 2; (n) substitution of isoleucine (I) corresponding to the I at position 262 of SEQ ID NO: 1 or position 310 of SEQ ID NO: 2;
(o) substitution of aspartic acid (D) corresponding to the D at position 267 of SEQ ID NO: 1 or position 315 of SEQ ID NO: 2;
(p) substitution of methionine (M) corresponding to the M at position 275 of SEQ ID NO: 1 or position 323 of SEQ ID NO: 2;
(q) substitution of isoleucine (I) corresponding to the I at position 298 of SEQ ID NO: 1 or position 346 of SEQ ID NO: 2;
(r) substitution of valine (V) corresponding to the V at position 300 of SEQ ID NO: 1 or position 348 of SEQ ID NO: 2;
(s) substitution of glycine (G) corresponding to the G at position 301 of SEQ ID NO: 1 or position 349 of SEQ ID NO: 2;
(t) substitution of serine (S) corresponding to the S at position 305 of SEQ ID NO: 1 or position 353 of SEQ ID NO: 2;
(u) substitution of phenylalanine (F) corresponding to the F at position 306 of SEQ ID NO: 1 or position 354 of SEQ ID NO: 2;
(v) substitution of leucine (L) corresponding to the L at position 307 of SEQ ID NO: 1 or position 355 of SEQ ID NO: 2;
(w) substitution of tryptophan (W) corresponding to the W at position 308 of SEQ ID NO: 1 or position 356 of SEQ ID NO: 2;
(x) substitution of threonine (T) corresponding to the T at position 313 of SEQ ID NO: 1 or position 361 of SEQ ID NO: 2;
(y) substitution of serine (S) corresponding to the S at position 330 of SEQ ID NO: 1 or position 378 of SEQ ID NO: 2; and
(z) substitution of leucine (L) corresponding to the L at position 342 of SEQ ID NO: 1 or position 390 of SEQ ID NO: 2. In some implementations, the engineered bakuchiol-producing enzyme may comprise 2, 3, 4, or 5 or more amino acid substitutions. In some implementations, the engineered bakuchiol-producing enzyme may additionally comprise an N-terminal deletion of 1-120 amino acids.

The present disclosure additionally provides an engineered enzyme that catalyzes production of bakuchiol, exhibits prenyltransferase activity, or both, wherein the engineered enzyme comprises at least one substitution mutation selected from:
(a) substitution of phenylalanine (F) at position 42 of SEQ ID NO: 1 or position 90 of SEQ ID NO: 2;
(b) substitution of aspartate (D) at position 59 of SEQ ID NO: 1 or position 107 of SEQ ID NO: 2;
(c) substitution of leucine (L) at position 96 of SEQ ID NO: 1 or position 144 of SEQ ID NO: 2;
(d) substitution of histidine (H) at position 150 of SEQ ID NO: 1 or position 198 of SEQ ID NO: 2;
(e) substitution of valine (V) at position 173 of SEQ ID NO: 1 or position 221 of SEQ ID NO: 2;
(f) substitution of glycine (G) at position 187 of SEQ ID NO: 1 or position 235 of SEQ ID NO: 2;
(g) substitution of leucine (L) or valine (V) at position 193 of SEQ ID NO: 1 or position 241 of SEQ ID NO: 2;
(h) substitution of tyrosine (Y) at position 194 of SEQ ID NO: 1 or position 242 of SEQ ID NO: 2;
(i) substitution of serine (S), cysteine (C), valine (V), threonine (T), tyrosine (Y), arginine (R), methionine (M), or glutamine (Q) at position 197 of SEQ ID NO: 1 or position 245 of SEQ ID NO: 2;
(j) substitution of methionine (M) at position 214 of SEQ ID NO: 1 or position 262 of SEQ ID NO: 2;
(k) substitution of glutamine (Q) at position 222 of SEQ ID NO: 1 or position 270 of SEQ ID NO: 2;
(l) substitution of glutamate (E) at position 245 of SEQ ID NO: 1 or position 293 of SEQ ID NO: 2;
(m) substitution of arginine (R) at position 257 of SEQ ID NO: 1 or position 305 of SEQ ID NO: 2;
(n) substitution of leucine (L) at position 262 of SEQ ID NO: 1 or position 310 of SEQ ID NO: 2;
(o) substitution of cysteine (C), lysine (K), arginine (R), methionine (M), or leucine (L) at position 267 of SEQ ID NO: 1 or position 315 of SEQ ID NO: 2;
(p) substitution of valine (V), phenylalanine (F), or tyrosine (Y) at position 275 of SEQ ID NO: 1 or position 323 of SEQ ID NO: 2;
(q) substitution of valine (V) at position 298 of SEQ ID NO: 1 or position 346 of SEQ ID NO: 2;
(r) substitution of tryptophan (W), alanine (A), phenylalanine (F), glycine (G), tyrosine (Y), cysteine (C), or leucine (L) at position 300 of SEQ ID NO: 1 or position 348 of SEQ ID NO: 2;
(s) substitution of isoleucine (I) at position 301 of SEQ ID NO: 1 or position 349 of SEQ ID NO: 2;
(t) substitution of proline (P) or isoleucine (I) at position 305 of SEQ ID NO: 1 or position 353 of SEQ ID NO: 2;
(u) substitution of arginine (R) or glycine (G) at position 306 of SEQ ID NO: 1 or position 354 of SEQ ID NO: 2;
(v) substitution of proline (P) at position 307 of SEQ ID NO: 1 or position 355 of SEQ ID NO: 2;
(w) substitution of aspartate (D) at position 308 of SEQ ID NO: 1 or position 356 of SEQ ID NO: 2;
(x) substitution of glycine (G) at position 313 of SEQ ID NO: 1 or position 361 of SEQ ID NO: 2;
(y) substitution of glycine (G) at position 330 of SEQ ID NO: 1 or position 378 of SEQ ID NO: 2; and
(z) substitution of phenylalanine (F) at position 342 of SEQ ID NO: 1 or position 390 of SEQ ID NO: 2. In some implementations, the engineered bakuchiol-producing enzyme may comprise 2, 3, 4, or 5 or more amino acid substitutions. In some implementations, the engineered bakuchiol-producing enzyme may additionally comprise an N-terminal deletion of 1-120 amino acids.

For the purposes of the present disclosure, it is generally expected that the disclosed engineered bakuchiol-producing enzyme comprise a substitution mutation, an N-terminal deletion, or both that increases catalyzation of production of bakuchiol, prenyltransferase activity, or both, relative to a non-engineered enzyme comprising the same amino acid sequence but without the substitution mutation, N-terminal deletion, or both. For example, an engineered bakuchiol-producing enzyme, as described herein, catalyzes production of bakuchiol, exhibits prenyltransferase activity, or both, and the enzyme comprises nine transmembrane domains and loops connecting the transmembrane domains. However, the engineered enzyme comprises at least one substitution mutation on an internal loop or an external loop of the enzyme. Such an enzyme may further comprise an N-terminus and a C-terminus, and, in some implementations no amino acids are substituted in the first 1-75, 1-50, or 1-25 amino acids of the N-terminus or the last 1-75, 1-50, or 1-25 amino acids of the C-terminus. In some implementations, no amino acids are substituted in the first 50 amino acids of the N-terminus or the last 50 amino acids of the C-terminus. In some implementations, the engineered enzyme may further comprise an N-terminal deletion as described herein.

In some implementations, a variant as described herein exhibits increased bakuchiol-producing activity relative to the wild-type BAK28 (SEQ ID NO: 1) or BAK36 (SEQ ID NO: 2) enzymes, such that its activity is at least about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, about 500%, about 550%, about 600%, about 650%, about 700%, about 750%, about 800%, about 850%, about 900%, about 950%, about 1000%, about 1100%, about 1200%, about 1300%, about 1400%, about 1500%, about 1600%, about 1700%, about 1800%, about 1900%, about 2000%, about 2250%, about 2500%, about 2750%, about 3000%, about 3250%, about 3500%, about 3750%, about 4000%, about 4250%, about 4500%, about 4750%, about 5000%, about 10000%, about 100000%, about 1000000%, about 10000000%, or about 20000000% or more than that of the wild-type BAK28 or BAK36 enzymes, as determined by a measure of bakuchiol production or titer.

In some implementations, a variant as described herein exhibits increased bakuchiol-producing activity relative to the wild-type BAK28 (SEQ ID NO: 1) or BAK36 (SEQ ID NO: 2) enzymes, such that its activity is at least about 2-fold—e.g., at least about 4-fold, about 5-fold, about 10-fold, about 18-fold, about 20-fold, about 50-fold, about 100-fold, about 200-fold, about 1000-fold, about 5000-fold, about 10000-fold, about 20000-fold, about 50000-fold, about 100000-fold, about 200000-fold, about 500000-fold, or about 1000000-fold, or more, than that of the wild-type BAK28 or BAK36 enzymes, as determined by a measure of bakuchiol production or titer.

### VI. Host Cells and Transgenic Cells

Bioproduction of bakuchiol can rely on a host cell that expresses a bakuchiol-producing protein as disclosed herein or a transgenic cell that expresses a bakuchiol-producing protein as disclosed herein. A host cell may or may not natively express the bakuchiol-producing protein. A transgenic cell may be a cell that comprises a transgene encoding a bakuchiol-producing protein. In some implementations, a transgene encoding a bakuchiol-producing protein may enable the transgenic cell to express the bakuchiol-producing protein.

The present disclosure provides an engineered host cell or a transgenic cell that expresses any of the disclosed bakuchiol-producing proteins. In one aspect, the present disclosure provides a transgenic cell that comprises a transgene encoding any of the disclosed bakuchiol-producing proteins. In some implementations, the engineered host cell or transgenic cell may comprise a bakuchiol-producing protein that has at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 1. In some implementations, the engineered host cell or transgenic cell may comprise a bakuchiol-producing protein that has at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 2. In some implementations, the bakuchiol-producing protein expressed by the engineered host cell or transgenic cell may share at least 90% identity with SEQ ID NO: 1 or SEQ ID NO: 2. In some implementations, the bakuchiol-producing protein expressed by the engineered host cell or transgenic cell may share at least 95% identity with SEQ ID NO: 1 or SEQ ID NO: 2. In some implementations, the bakuchiol-producing protein expressed by the engineered host cell or transgenic cell may share at least 99% identity with SEQ ID NO: 1 or SEQ ID NO: 2. Thus, this disclosure encompasses expression of proteins with varying degrees of sequence identity compared to SEQ ID NO: 1 and SEQ ID NO: 2, so long as the protein exhibits prenyltransferase activity, catalyzes the production of bakuchiol, or both.

In some implementations, the engineered host cell or transgenic cell may comprise a bakuchiol-producing protein that has at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 3. In some implementations, the bakuchiol-producing protein expressed by the engineered host cell or transgenic cell may share at least 90% identity with SEQ ID NO: 3. In some implementations, the bakuchiol-producing protein expressed by the engineered host cell or transgenic cell may share at least 95% identity with SEQ ID NO: 3. In some implementations, the bakuchiol-producing protein expressed by the engineered host cell or transgenic cell may share at least 99% identity with SEQ ID NO: 3. Thus, this disclosure encompasses expression of proteins with varying degrees of sequence identity compared to SEQ ID NO: 3, so long as the protein exhibits prenyltransferase activity, catalyzes the production of bakuchiol, or both.

In some implementations, an engineered host cell or transgenic cell of the present disclosure can express a bakuchiol-producing protein comprising SEQ ID NO: 1 (i.e., it is 361 amino acids or longer). In some implementations, an engineered host cell or transgenic cell of the present disclosure can express a bakuchiol-producing protein comprising 361 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 1. In some implementations, an engineered host cell or transgenic cell of the present disclosure can express a bakuchiol-producing protein consisting of SEQ ID NO: 1. In some implementations, an engineered host cell or transgenic cell of the present disclosure can express a bakuchiol-producing protein consisting of 361 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 1. In some implementations, an engineered host cell or transgenic cell of the present disclosure can express a bakuchiol-producing protein comprising about 365, about 370, about 375, about 380, about 385, about 390, about 395, about 400, about 405, about 410, about 415, about 420, about 425, about 430, about 435, about 440, or about 450 amino acids, wherein at least about 361 amino acids of the protein have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 1. Varying degrees of sequence identity and coverage are acceptable and are included as part of the implementations herein, so long as the protein exhibits prenyltransferase activity, catalyzes the production of bakuchiol, or both.

In some implementations, an engineered host cell or transgenic cell of the present disclosure can express a bakuchiol-producing protein comprising SEQ ID NO: 2 (i.e., it is 409 amino acids or longer). In some implementations, an engineered host cell or transgenic cell of the present disclosure can express a bakuchiol-producing protein comprising 409 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 2. In some implementations, an engineered host cell or transgenic cell of the present disclosure can express a bakuchiol-producing protein consisting of SEQ ID NO: 2. In some implementations, an engineered host cell or transgenic cell of the present disclosure can express a bakuchiol-producing protein consisting of 409 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 2. In some implementations, an engineered host cell or transgenic cell of the present disclosure can express a bakuchiol-producing protein comprising about 410, about 415, about 420, about 425, about 430, about 435, about 440, or about 450 amino acids, wherein at least about 409 amino acids of the protein have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 2. Varying degrees of sequence identity and coverage are acceptable and are included as part of the implementations herein, so long as the protein exhibits prenyltransferase activity, catalyzes the production of bakuchiol, or both.

In some implementations, an engineered host cell or transgenic cell of the present disclosure can express a bakuchiol-producing protein that is shorter than SEQ ID NO: 1 or SEQ ID NO: 2. For example, the expressed bakuchiol-producing protein may be less than 409 or less than 361 amino acids in length, so long as the protein has a catalytic domain that has at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 1 or SEQ ID NO: 2. In some implementations at least about 50, at least about 75, at least about 100, at least about 125, at least about 150, at least about 175, at least about 200, at least about 225, at least about 250, at least about 275, at least about 300, at least about 325, or at least about 350 amino acids of the protein can have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 1 or SEQ ID NO: 2. Varying degrees of sequence identity and coverage are acceptable and are included as part of the implementations herein, so long as the protein exhibits prenyltransferase activity, catalyzes the production of bakuchiol, or both.

In some implementations, an engineered host cell or transgenic cell of the present disclosure can express a bakuchiol-producing protein comprising SEQ ID NO: 3 (i.e., it is 373 amino acids or longer). In some implementations, an engineered host cell or transgenic cell of the present disclosure can express a bakuchiol-producing protein comprising 373 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 3. In some implementations, an engineered host cell or transgenic cell of the present disclosure can express a bakuchiol-producing protein consisting of SEQ ID NO: 3. In some implementations, an engineered host cell or transgenic cell of the present disclosure can express a bakuchiol-producing protein consisting of 373 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 3. In some implementations, an engineered host cell or transgenic cell of the present disclosure can express a bakuchiol-producing protein comprising about 370, about 375, about 380, about 385, about 390, about 395, about 400, about 405, about 410, about 415, about 420, about 425, about 430, about 435, about 440, or about 450 amino acids, wherein at least about 373 amino acids of the protein have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 3. Varying degrees of sequence identity and coverage are acceptable and are included as part of the implementations herein, so long as the protein exhibits prenyltransferase activity, catalyzes the production of bakuchiol, or both.

In some implementations, an engineered host cell or transgenic cell of the present disclosure can express a bakuchiol-producing protein that is shorter than SEQ ID NO: 3. For example, the expressed bakuchiol-producing protein may be less than 373 amino acids in length, so long as the protein has a catalytic domain that has at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 3. In some implementations at least about 50, at least about 75, at least about 100, at least about 125, at least about 150, at least about 175, at least about 200, at least about 225, at least about 250, at least about 275, at least about 300, at least about 325, or at least about 350 amino acids of the protein can have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 3. Varying degrees of sequence identity and coverage are acceptable and are included as part of the implementations herein, so long as the protein exhibits prenyltransferase activity, catalyzes the production of bakuchiol, or both.

In some implementations, an engineered host cell or transgenic cell of the present disclosure can express a bakuchiol-producing protein comprising any one of SEQ ID NO: 4-51 (i.e., it is 373 amino acids or longer). In some implementations, an engineered host cell or transgenic cell of the present disclosure can express a bakuchiol-producing protein comprising 373 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with any one of SEQ ID NO: 4-51. In some implementations, an engineered host cell or transgenic cell of the present disclosure can express a bakuchiol-producing protein consisting of any one of SEQ ID NO: 4-51. In some implementations, an engineered host cell or transgenic cell of the present disclosure can express a bakuchiol-producing protein consisting of 373 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with any one of SEQ ID NO: 4-51. In some implementations, an engineered host cell or transgenic cell of the present disclosure can express a bakuchiol-producing protein comprising about 370, about 375, about 380, about 385, about 390, about 395, about 400, about 405, about 410, about 415, about 420, about 425, about 430, about 435, about 440, or about 450 amino acids, wherein at least about 373 amino acids of the protein have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with any one of SEQ ID NO: 4-51. Varying degrees of sequence identity and coverage are acceptable and are included as part of the implementations herein, so long as the protein exhibits prenyltransferase activity, catalyzes the production of bakuchiol, or both.

In some implementations, an engineered host cell or transgenic cell of the present disclosure can express a bakuchiol-producing protein that is shorter than any one of SEQ ID NO: 4-51. For example, the expressed bakuchiol-producing protein may be less than 373 amino acids in length, so long as the protein has a catalytic domain that has at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with any one of SEQ ID NO: 4-51. In some implementations at least about 50, at least about 75, at least about 100, at least about 125, at least about 150, at least about 175, at least about 200, at least about 225, at least about 250, at least about 275, at least about 300, at least about 325, or at least about 350 amino acids of the protein can have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with any one of SEQ ID NO: 4-51. Varying degrees of sequence identity and coverage are acceptable and are included as part of the implementations herein, so long as the protein exhibits prenyltransferase activity, catalyzes the production of bakuchiol, or both.

Various prokaryotic and eukaryotic expression systems are commonly used for bioproduction, though factors including the growth conditions, type of fermenter utilized, toxicity (if any) of the product, and other metabolic considerations of the microbe producing the product of interest may be employed to select a suitable system. Thus, in some implementations, a host cell or a transgenic cell suitable for expressing the disclosed bakuchiol-producing proteins may be a prokaryote. In in some implementations, a host cell or a transgenic cell suitable for expressing the disclosed bakuchiol-producing proteins may be a eukaryote.

In some implementations, the engineered host cell or transgenic cell is a prokaryote. Model prokaryotic systems that may be utilized as a transgenic cell include but are not limited to Escherichia coli (E. coli), an Acinetobacter species, a Pseudomonas species, a Streptomyces species, and a Mycobacterium species. Additional suitable prokaryotic expression systems include, but are not limited to, Klebsiella, Lactococcus, Mannheimia, Corynebacterium, Vibrio, and Bacillis.

In some implementations, the engineered host cell or transgenic cell is a eukaryote. Model eukaryotic systems that may be utilized as a transgenic cell include but are not limited to Saccharomyces cerevisiae (S. cerevisiae) or other yeast species; a filamentous fungi, optionally selected from an Aspergillus species and a Trichoderma species; an algae, optionally selected from Botryococcus braunii, Chlorella sp., Crypthecodinium cohnii , Cylindrotheca sp., Nitzschia sp., Phaeodactylum tricornutum, Schizochytrium sp., and Tetraselmis suecia; and an amoeba, which is optionally Dictyostehum discoideum. Additional suitable eukaryotic expression systems include, but are not limited to, Pichia pastoris, Yarrowia lipolytica, Kluyveromyces marxianus, Rhodosporidium toruloides. Aspergillus (oryzae, nidulans, niger), Trichoderma reesei, and Penicillium chrysogenum.

In some implementations, for the engineered host cells and transgenic cells of the present disclosure bakuchiol is produced when the cell is cultured in the presence of p-coumaric acid and (i) geranyl pyrophosphate (GPP), (ii) dimethylallyl pyrophosphate (DMAPP), (iii) isopentenyl pyrophosphate (IPP), or any combination of (i)-(iii) thereof. The amount of bakuchiol produced may vary. For example, an engineered host cell or a transgenic cell of the present disclosure may produce at least about 0.1 μg/L—e.g., at least about 0.2 μg/L, at least about 0.3 μg/L, at least about 0.4 μg/L, at least about 0.5 μg/L, at least about 0.6 μg/L, at least about 0.7 μg/L, at least about 0.8 μg/L, at least about 0.9 μg/L, at least about 1.0 μg/L, at least about 1.1 μg/L, at least about 1.2 μg/L, at least about 1.3 μg/L, at least about 1.4 μg/L, at least about 1.5 μg/L, at least about 1.6 μg/L, at least about 1.7 μg/L, at least about 1.8 μg/L, at least about 1.9 μg/L, at least about 2.0 μg/L, at least about 2.1 μg/L, at least about 2.2 μg/L, at least about 2.3 μg/L, at least about 2.4 μg/L, at least about 2.5 μg/L, or more, of bakuchiol within about 48 hours of culture. Longer or shorter periods of culture time are also possible. For the purposes of the disclosed compositions and methods, it is understood that in some implementations p-coumaric acid, GPP, DMAPP, IPP, or all or a combination thereof may be produced endogenously by the host cell or transgenic cell, and do not require exogenous addition into, for example, the cell culture medium. In some implementations, exogenous p-coumaric acid, GPP, DMAPP, IPP, or all or a combination thereof may be added to the culture medium.

As noted above, in implementations involving a transgenic cell (e.g., S. cerevisiae or E. coli), the transgenic cell will comprise a transgene encoding the bakuchiol-producing protein, and the transgene can be integrated into the transgenic cell's genome. The transgene may be integrated within an expression cassette that appropriately drives expression of the bakuchiol-producing protein. For those implementations in which genome integration of the transgene is preferred or desired, known methods of integration can be used, including but not limited to Cas-based systems (e.g., Cas9, Cas12, etc.), homologous recombination, gene gun, conjugation protocols, lambda red, etc. Alternatively, in some implementations, the transgene may not be integrated into the genome, and instead may express the bakuchiol-producing protein from, for example, a plasmid or similar vector.

An expression cassette or vector for expressing the transgene may comprise a promoter and a terminator. Suitable promoters that can be used may include but are not limited to GAL1, TEF2, TEF1, TDH3, ENO2, CCW12, EF-1a promoter, CMV immediate early, HSV thymidine kinase, early and late SV40, LTRs from retrovirus, and mouse metallothionein-I. In some implementations, the promoter is GAL1. In some implementations, an inducible or repressible promoter, such as GAL1, GAL2, GAL7, GAL10, CUP1, MET3, MET17, or MET25, may be used. Inducible promoters operably link the expression of a target gene (e.g., the nucleic acid sequence encoding a bakuchiol-producing protein) to a specific signal or a particular biotic or abiotic factor. Types of inducible promoters that may be utilized in the disclosed include, but are not limited to, chemically-inducible promoters (i.e., antibiotics, steroids, metals, etc.), light-inducible promoters, heat-inducible promoters, and hypoxia-inducible promoters. Transcription terminators that may be used are also known in the art (see Bittner et al., Methods in Enzymol. 153: 516-544 (1987)), and include but are not limited to GAT2, Rho-dependent terminators, Rho-independent terminators, poly-A sequences, and the like. In some implementations, the terminator is GAT2.

### VII. Methods of Bioproduction and Batches Produced Therefrom

The identification, isolation, and characterization of previously unknown bakuchiol-producing prenyltransferase enzymes allows methods of bioproduction of bakuchiol. Thus, the present disclosure provides methods of producing bakuchiol, comprising culturing an engineered host cell or a transgenic cell disclosed herein in a culture medium and in the presence of p-coumaric acid and geranyl pyrophosphate (GPP), dimethylallyl pyrophosphate (DMAPP), isopentenyl pyrophosphate (IPP), or any combination of GPP, DMAPP, and IPP. For the purposes of the disclosed methods, it is understood that in some implementations p-coumaric acid, GPP, DMAPP, IPP, or all or a combination thereof may be produced endogenously by the host cell or transgenic cell, and do not require exogenous addition into, for example, the cell culture medium. In some implementations, exogenous p-coumaric acid, GPP, DMAPP, IPP, or all or a combination thereof may be added to the culture medium.

In some implementations, the methods comprise culturing a transgenic cell (e.g., S. cerevisiae or E. coli) comprising a transgene that encodes a bakuchiol-producing protein that has at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 1; or at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 2; or at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 3; or at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with any one of SEQ ID NO: 4-51. In some implementations, the bakuchiol-producing protein expressed by the transgenic cell may share at least 90% identity with SEQ ID NO: 1, SEQ ID NO: 2, SEQ ID NO: 3, or any one of SEQ ID NO: 4-51. In some implementations, the bakuchiol-producing protein expressed by the transgenic cell may share at least 95% identity with SEQ ID NO: 1, SEQ ID NO: 2, SEQ ID NO: 3, or any one of SEQ ID NO: 4-51. In some implementations, the bakuchiol-producing protein expressed by the transgenic cell may share at least 99% identity with SEQ ID NO: 1, SEQ ID NO: 2, SEQ ID NO: 3, or any one of SEQ ID NO: 4-51. Thus, the protein may possess varying degrees of sequence identity compared to SEQ ID NOs: 1-41, so long as the protein exhibits prenyltransferase activity, catalyzes the production of bakuchiol, or both.

In some implementations, the methods comprise culturing a transgenic cell comprising a transgene encoding a bakuchiol-producing protein comprising SEQ ID NO: 1 (i.e., it is 361 amino acids or longer). In some implementations, the methods comprise culturing a transgenic cell comprising a transgene encoding a bakuchiol-producing protein comprising 361 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 1. In some implementations, the methods comprise culturing a transgenic cell comprising a transgene encoding a bakuchiol-producing protein consisting of SEQ ID NO: 1. In some implementations, the methods comprise culturing a transgenic cell comprising a transgene encoding a bakuchiol-producing protein consisting of 361 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 1. In some implementations, the methods comprise culturing a transgenic cell comprising a transgene encoding a bakuchiol-producing protein comprising about 365, about 370, about 375, about 380, about 385, about 390, about 395, about 400, about 405, about 410, about 415, about 420, about 425, about 430, about 435, about 440, or about 450 amino acids, wherein at least about 361 amino acids of the protein have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 1. Varying degrees of sequence identity and coverage may be employed, so long as the protein exhibits prenyltransferase activity, catalyzes the production of bakuchiol, or both.

In some implementations, the methods comprise culturing a transgenic cell comprising a transgene encoding a bakuchiol-producing protein comprising SEQ ID NO: 2 (i.e., it is 409 amino acids or longer). In some implementations, the methods comprise culturing a transgenic cell comprising a transgene encoding a bakuchiol-producing protein comprising 409 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 2. In some implementations, the methods comprise culturing a transgenic cell comprising a transgene encoding a bakuchiol-producing protein consisting of SEQ ID NO: 2. In some implementations, the methods comprise culturing a transgenic cell comprising a transgene encoding a bakuchiol-producing protein consisting of 409 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 2. In some implementations, the methods comprise culturing a transgenic cell comprising a transgene encoding a bakuchiol-producing protein comprising about 410, about 415, about 420, about 425, about 430, about 435, about 440, or about 450 amino acids, wherein at least about 409 amino acids of the protein have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 2. Varying degrees of sequence identity and coverage may be employed, so long as the protein exhibits prenyltransferase activity, catalyzes the production of bakuchiol, or both.

In some implementations, the methods comprise culturing a transgenic cell comprising a transgene encoding a bakuchiol-producing protein comprising SEQ ID NO: 3 (i.e., it is 373 amino acids or longer). In some implementations, the methods comprise culturing a transgenic cell comprising a transgene encoding a bakuchiol-producing protein comprising 373 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 3. In some implementations, the methods comprise culturing a transgenic cell comprising a transgene encoding a bakuchiol-producing protein consisting of SEQ ID NO: 3. In some implementations, the methods comprise culturing a transgenic cell comprising a transgene encoding a bakuchiol-producing protein consisting of 373 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 3. In some implementations, the methods comprise culturing a transgenic cell comprising a transgene encoding a bakuchiol-producing protein comprising about 375, about 380, about 385, about 390, about 395, about 400, about 405, about 410, about 415, about 420, about 425, about 430, about 435, about 440, or about 450 amino acids, wherein at least about 373 amino acids of the protein have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 3. Varying degrees of sequence identity and coverage may be employed, so long as the protein exhibits prenyltransferase activity, catalyzes the production of bakuchiol, or both.

In some implementations, the methods comprise culturing a transgenic cell comprising a transgene encoding a bakuchiol-producing protein comprising any one of SEQ ID NO: 4-51 (i.e., it is 373 amino acids or longer). In some implementations, the methods comprise culturing a transgenic cell comprising a transgene encoding a bakuchiol-producing protein comprising 373 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with any one of SEQ ID NO: 4-51. In some implementations, the methods comprise culturing a transgenic cell comprising a transgene encoding a bakuchiol-producing protein consisting of any one of SEQ ID NO: 4-51. In some implementations, the methods comprise culturing a transgenic cell comprising a transgene encoding a bakuchiol-producing protein consisting of 373 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with any one of SEQ ID NO: 4-51. In some implementations, the methods comprise culturing a transgenic cell comprising a transgene encoding a bakuchiol-producing protein comprising about 375, about 380, about 385, about 390, about 395, about 400, about 405, about 410, about 415, about 420, about 425, about 430, about 435, about 440, or about 450 amino acids, wherein at least about 373 amino acids of the protein have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with any one of SEQ ID NO: 4-51. Varying degrees of sequence identity and coverage may be employed, so long as the protein exhibits prenyltransferase activity, catalyzes the production of bakuchiol, or both.

In some implementations, the methods comprise culturing a transgenic cell comprising a transgene encoding a bakuchiol-producing protein that is shorter than SEQ ID NO: 1 or SEQ ID NO: 2. For example, the expressed bakuchiol-producing protein may be less than 409 or less than 361 amino acids in length, so long as the protein has a catalytic domain that has at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 1 or SEQ ID NO: 2. In some implementations at least about 50, at least about 75, at least about 100, at least about 125, at least about 150, at least about 175, at least about 200, at least about 225, at least about 250, at least about 275, at least about 300, at least about 325, or at least about 350 amino acids of the protein can have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 1 or SEQ ID NO: 2. Varying degrees of sequence identity and coverage may be employed, so long as the protein exhibits prenyltransferase activity, catalyzes the production of bakuchiol, or both.

In some implementations, the methods comprise culturing a transgenic cell comprising a transgene encoding a bakuchiol-producing protein that is shorter than SEQ ID NO: 3. For example, the expressed bakuchiol-producing protein may be less than 373 amino acids in length, so long as the protein has a catalytic domain that has at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 3. In some implementations at least about 50, at least about 75, at least about 100, at least about 125, at least about 150, at least about 175, at least about 200, at least about 225, at least about 250, at least about 275, at least about 300, at least about 325, or at least about 350 amino acids of the protein can have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 3. Varying degrees of sequence identity and coverage may be employed, so long as the protein exhibits prenyltransferase activity, catalyzes the production of bakuchiol, or both.

In some implementations, the methods comprise culturing a transgenic cell comprising a transgene encoding a bakuchiol-producing protein that is shorter than any one of SEQ ID NO: 4-51. For example, the expressed bakuchiol-producing protein may be less than 373 amino acids in length, so long as the protein has a catalytic domain that has at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with any one of SEQ ID NO: 4-51. In some implementations at least about 50, at least about 75, at least about 100, at least about 125, at least about 150, at least about 175, at least about 200, at least about 225, at least about 250, at least about 275, at least about 300, at least about 325, or at least about 350 amino acids of the protein can have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with any one of SEQ ID NO: 4-51. Varying degrees of sequence identity and coverage may be employed, so long as the protein exhibits prenyltransferase activity, catalyzes the production of bakuchiol, or both.

Various prokaryotic and eukaryotic expression systems can be utilized for the disclosed methods. In some implementations, the microbial cell used in the methods may be a prokaryote, including but are not limited to Escherichia coli (E. coli), an Acinetobacter species, a Pseudomonas species, a Streptomyces species, and a Mycobacterium species. Additionally suitable prokaryotic expression systems include, but are not limited to, Klebsiella, Lactococcus, Mannheimia, Corynebacterium, Vibrio, and Bacillis. In in some implementations, the transgenic cell used in the methods may be a eukaryote, including but are not limited to Saccharomyces cerevisiae (S. cerevisiae) or other yeast species; a filamentous fungi, optionally selected from an Aspergillus species and a Trichoderma species; an algae, optionally selected from Botryococcus braunii, Chlorella sp., Crypthecodinium cohnii, Cylindrotheca sp., Nitzschia sp., Phaeodactylum tricornutum, Schizochytrium sp., and Tetraselmis suecia; and an amoeba, which is optionally Dictyostelium discoideum. Additional suitable eukaryotic expression systems include, but are not limited to, Pichia pastoris, Yarrowia hpolytica, Kluyveromyces marxianus, Rhodosporidium toruloides. Aspergillus (oryzae, nidulans, niger), Trichoderma reesei, and Penicillium chrysogenum.

The disclosed methods can be carried out in a bioproduction reactor, fermentation tank, culture flask, or other suitable containers for bioproduction. Various different culture mediums can be selected based on the particular transgenic species used and the growth conditions, among other things. In some implementations, minimal culture medium may be supplemented as needed to optimize growth and production of a given transgenic cell type. For example, in some implementations, such as those utilizing transgenic S. cerevisiae, the culture medium may comprise about 3% w/v maltodextrin, about 0.2% w/v glucose, alpha-amylase, or any combination thereof.

As discussed above, and without being bound by any particular theory, it is believed that bioproduction of bakuchiol is catalyzed through a mechanism involved p-coumaric acid and geranyl pyrophosphate (GPP), dimethylallyl pyrophosphate (DMAPP), isopentenyl pyrophosphate (IPP), or any combination of GPP, DMAPP, and IPP. Thus, in some implementations the culture medium used for the disclosed methods may optionally include some p-coumaric acid to supplement that which is endogenously produced by a given transgenic cell or host cell. Indeed, In some implementations p-coumaric acid may be produced endogenously by the host cell or transgenic cell and the culture medium is not supplemented. In some implementations, the culture medium may comprise at least about 1.50 mM p-coumaric acid—e.g., at least about 1.75 mM p-coumaric acid, at least about 2.00 p-coumaric acid, at least about 2.25 mM p-coumaric acid, at least about 2.50 mM p-coumaric acid, at least about 2.75 mM p-coumaric acid, at least about 3.00 p-coumaric acid, at least about 3.25 mM p-coumaric acid, at least about 3.50 mM p-coumaric acid, at least about 3.75 mM p-coumaric acid, at least about 4.00 p-coumaric acid, or more.

The disclosed methods are the first to achieve production of bakuchiol in by a transgenic organism. These methods of bioproduction may be further optimized and developed to increase yield. For example, in some implementations, the disclosed methods may produce at least about 0.1 μg/L, at least about 0.2 μg/L, at least about 0.3 μg/L, at least about 0.4 μg/L, at least about 0.5 μg/L, at least about 0.6 μg/L, at least about 0.7 μg/L, at least about 0.8 μg/L, at least about 0.9 μg/L, at least about 1.0 μg/L, at least about 1.1 μg/L, at least about 1.2 μg/L, at least about 1.3 μg/L, at least about 1.4 μg/L, at least about 1.5 μg/L, at least about 1.6 μg/L, at least about 1.7 μg/L, at least about 1.8 μg/L, at least about 1.9 μg/L, at least about 2.0 μg/L, at least about 2.1 μg/L, at least about 2.2 μg/L, at least about 2.3 μg/L, at least about 2.4 μg/L, at least about 2.5 μg/L, at least about 3.0 μg/L, at least about 3.5 μg/L, at least about 4.0 μg/L, at least about 4.5 μg/L, at least about 5.0 μg/L, at least about 5.5 μg/L, at least about 6.0 μg/L, at least about 6.5 μg/L, at least about 7.0 μg/L, at least about 7.5 μg/L, at least about 8.0 μg/L, at least about 8.5 μg/L, at least about 9.0 μg/L, at least about 9.5 μg/L, at least about 10.0 μg/L, at least about 20 μg/L, at least about 30 μg/L, at least about 40 μg/L, at least about 50 μg/L, at least about 75 μg/L, at least about 100 μg/L, or more of bakuchiol within at least about 6 hours, at least about 12 hours, at least about 18 hours, at least about 24 hours, at least about 36 hours, or at least about 48 hours of culture. In some implementations, the disclosed methods may produce at least about 0.1 μg/L, at least about 0.2 μg/L, at least about 0.3 μg/L, at least about 0.4 μg/L, at least about 0.5 μg/L, at least about 0.6 μg/L, at least about 0.7 μg/L, at least about 0.8 μg/L, at least about 0.9 μg/L, at least about 1.0 μg/L, at least about 1.1 μg/L, at least about 1.2 μg/L, at least about 1.3 μg/L, at least about 1.4 μg/L, at least about 1.5 μg/L, at least about 1.6 μg/L, at least about 1.7 μg/L, at least about 1.8 μg/L, at least about 1.9 μg/L, at least about 2.0 μg/L, at least about 2.1 μg/L, at least about 2.2 μg/L, at least about 2.3 μg/L, at least about 2.4 μg/L, at least about 2.5 μg/L, at least about 3.0 μg/L, at least about 3.5 μg/L, at least about 4.0 μg/L, at least about 4.5 μg/L, at least about 5.0 μg/L, at least about 5.5 μg/L, at least about 6.0 μg/L, at least about 6.5 μg/L, at least about 7.0 μg/L, at least about 7.5 μg/L, at least about 8.0 μg/L, at least about 8.5 μg/L, at least about 9.0 μg/L, at least about 9.5 μg/L, at least about 10.0 μg/L, at least about 20 μg/L, at least about 30 μg/L, at least about 40 μg/L, at least about 50 μg/L, at least about 75 μg/L, at least about 100 μg/L, or more of bakuchiol within about 6 hours of culture or less, about 12 hours of culture or less, about 18 hours of culture or less, about 24 hours of culture or less, about 36 hours of culture or less, or about 48 hours of culture or less.

The disclosed methods are the first to provide a process of bioproducing bakuchiol in batches that can be used for commercial consumption. This, the present disclosure provides batches of bakuchiol produced by the methods disclosed herein. A bioproduction batch of bakuchiol may have a chemical purity of at least 90%, at least 91%, at least 92%, at least 93%, at least 94%, at least 95%, at least 96%, at least 97%, at least 98%, at least 99%, or 100%, or any values in between any of the two aforementioned values, and no single impurity of greater than 1%, no greater than about 0.5%, or greater than about 0.1%. The level of impurities in a given batch of bakuchiol can be determined by high-performance liquid chromatography (HPLC) and other suitable techniques.

The physical state of the bakuchiol batch can vary as need and depending on the stage of the production process, and the disclosed batches may be solid or liquid. Liquid batches of bakuchiol may be in the form of a non-aqueous solution, such as an oil, an organic solvent, or an aqueous solution. The concentration of bakuchiol in a liquid batch (e.g., in an oil or aqueous solution) may be at least about 0.1 μg/L, at least about 0.2 μg/L, at least about 0.3 μg/L, at least about 0.4 μg/L, at least about 0.5 μg/L, at least about 0.6 μg/L, at least about 0.7 μg/L, at least about 0.8 μg/L, at least about 0.9 μg/L, at least about 1.0 μg/L, at least about 1.1 μg/L, at least about 1.2 μg/L, at least about 1.3 μg/L, at least about 1.4 μg/L, at least about 1.5 μg/L, at least about 1.6 μg/L, at least about 1.7 μg/L, at least about 1.8 μg/L, at least about 1.9 μg/L, at least about 2.0 μg/L, at least about 2.1 μg/L, at least about 2.2 μg/L, at least about 2.3 μg/L, at least about 2.4 μg/L, at least about 2.5 μg/L, at least about 3.0 μg/L, at least about 3.5 μg/L, at least about 4.0 μg/L, at least about 4.5 μg/L, at least about 5.0 μg/L, at least about 5.5 μg/L, at least about 6.0 μg/L, at least about 6.5 μg/L, at least about 7.0 μg/L, at least about 7.5 μg/L, at least about 8.0 μg/L, at least about 8.5 μg/L, at least about 9.0 μg/L, at least about 9.5 μg/L, at least about 10.0 μg/L, at least about 20 μg/L, at least about 30 μg/L, at least about 40 μg/L, at least about 50 μg/L, at least about 75 μg/L, at least about 100 μg/L, or more.

### VIII. Detection and Quantitation of Bakuchiol

The present disclosure provides methods of detecting bakuchiol and methods of quantifying bakuchiol using analytic techniques, including mass spectrometry. These methods may be useful for quality control of bakuchiol production by the disclosed bioproduction methods and any other known techniques of bakuchiol synthesis, extraction, or isolation.

In one implementation, bakuchiol can be detected by liquid chromatography mass spectrometry (LCMS) using, for example, an Agilent 1290 UHPLC and a 6460 triple-quadrupole mass spectrometer. Quantitation and compound identity can be determined by using an external standard curve of an authentic sample of bakuchiol.

Aqueous samples of bakuchiol can be diluted with isopropyl alcohol. In one implementation, the additional of isopropyl alcohol is not a purification process, per se, and the sample remains a 1-phase solution. However, the isopropyl alcohol may be extracting bakuchiol from hydrophobic surfaces such as lab ware and cellular membranes. The isopropyl alcohol may also help to clean the sample by precipitating proteins and other interfering material.

Beyond the addition of isopropyl alcohol, additional optional preparation processes include, but are not limited to extracting bakuchiol from the sample and centrifuging the sample to obtain a bakuchiol-containing supernatant.

Samples can be separated on a Waters BEH 50 mm×2.1 mM column, heated to 70° C., using water and acetonitrile mobile phases with a flow rate of 0.5 mL/min. The gradient may comprise of the following: 0 minutes 0% B, 1 minutes 99% B, 2 minutes 99% B, and 2.1 minutes 0% B. The gradient can utilize a linear ramp for transitions, and the process can be about 3 minutes long—e.g., about 2 minutes, about 2.5 minutes about 3 minutes, about 3.5 minutes, or about 4 minutes. A specific MRM can be used to detect bakuchiol in the mass spectrometry with an ESI source in the negative ion mode: Parent 255.2 m/z (unit), Product 172.1 m/z, Fragmenter 120V, Collision Energy 20 V, Cell Accelerator Voltage 5V with a 300 ms dwell time. Optical detection can also conducted at 260 nm with a 0.5 s response time.

Beyond this implementation, the present disclosure provides methods for determining an amount of bakuchiol in a sample by mass spectrometry, the method comprising:
(i) ionizing bakuchiol from the sample to generate one or more ions detectable by mass spectrometry;
(ii) determining an amount of bakuchiol ions by multiple reaction or high resolution accurate mass mass spectrometry; and
(iii) relating the amount of bakuchiol ions to the amount of bakuchiol in the sample, wherein a limit of detection of the method for bakuchiol is between about 0.001 μg/L and 0.0001 μg/L.

Various methods of ionization are known and can be utilized. For example, ionizing can comprise atmospheric pressure chemical ionization (APCI), electrospray ionization (ESI), or, if paired with gas chromatography, electron impact (EI) ionization Both APCI and ESI can be performed in negative ionization mode or positive ionization mode.

In some implementations, when using ESI in negative ion mode, the one or more ions (e.g., daughter ions after collision activation) may comprise an ion with a mass to charge ratio (m/z) of 172.1±0.5.

Prior to ionization, various methods of chromatography can be performs to isolate the bakuchiol and increase the sensitivity and selectivity of the mass spectroscopy. The chromatography may be liquid chromatography (LC) or gas chromatography (GC).prior to ionizing, the sample is subjected to liquid chromatography. Exemplary forms of LC that can be utilized include, but are not limited to, high performance liquid chromatography (HPLC), ultra performance liquid chromatography (UPLC), ultra high performance liquid chromatography (UHPLC), and supercritical fluid chromatography (SFC).

As discussed above, optional preparation processes that may be performed prior to ionizing, include diluting the sample with an alcohol (e.g., isopropyl alcohol), extracting the bakuchiol from the sample, centrifuging the sample to obtain the supernatant, or a combination thereof.

### NON-LIMITING WORKING EXAMPLES

The following examples are given to illustrate the present disclosure. It should be understood, however, that the disclosure is not to be limited to the specific conditions or details described in these examples.

### I. Example 1—Engineering of Bakuchiol-Producing Proteins and Bioproduction in Yeast

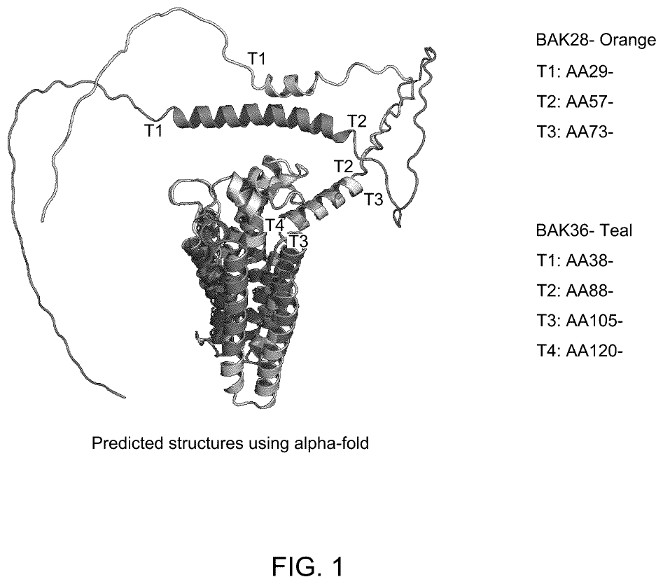

N-terminal trafficking sequences are common in plant enzymes. Many times, these N-terminal domains can become problematic when plant enzymes are expressed in heterologous organisms. To ensure that bakuchiol-producing enzyme engineering began with an optimized version of BAK28 and BAK36 for yeast expression, a series of N-terminal truncations were performed. Shown in FIG. 1 are the structures of BAK28 and BAK36 predicted by AlphaFold. N-terminal truncations of each of BAK28 and BAK36 were generated, resulting in truncation variants that begin with the indicated amino acid relative to SEQ ID NO: 1 (for BAK28) or SEQ ID NO: 2 (for BAK36). For example, BAK28 (T1) comprises an amino acid sequence that lacks the first 28 N-terminal amino acids of SEQ ID NO: 1 (T1:AA29−).

All putative prenyltranferase enzymes (referred to herein as “BAK genes”) were integrated into S. cerevisiae via standard LiAc chemical transformation methodologies using a Cas12-based system for directed nuclease-guided genomic integration. The BAK genes were expressed from the GAL80 locus, driven by a GAL1 promoter and GAT2 terminator unless otherwise noted in the genotype.

Resulting strains were grown and assayed at 30° C. in 96 mid-well plates with 3% w/v maltodextrin, 0.2% glucose defined medium (modified from Westfall 2012) with alpha-amylase for 24-48 hours, before transfer to the same medium with and 0-3 mM p-Coumaric Acid for 48 hours.

Primary bakuchiol screening was performed using Rapid Fire. Briefly, after incubation, samples were extracted by adding 500 uL of Isopropanol. Plates were shaken at 1,000 rpm for 15 minutes, then spun at 3,500×gravity for 5 minutes. 65 uL of sample was transferred. Bakuchiol primary screening was performed on the Agilent RapidFire with an Agilent 7010 Mass Spectrometer. Solid phase chromatography was performed using RapidFire C4 Type A columns. The injection cycle included a 1000 ms aspiration step, a 3000 ms load and wash step, 3500 ms elution step, and 750 ms recalibration step. Pump 1 used water, and Pumps 2 and 3 used 89% Acetonitrile, 10% IPA, 1% Water. 50 ng/mL 4-(4-chlorophenoxy) phenol was added as an internal standard.

The 255.2-172.1 transition was used for Bakuchiol; the 219.0-190.5 transition was used for 4-(4-chlorophenoxy) phenol.

Bakuchiol was quantified by LCMS using an Agilent 1290 UHF′LC and a 6460 triple-quadrupole mass spectrometer. Quantitation and compound identity were determined by using an external standard curve of an authentic sample of bakuchiol. Briefly, microfermentation samples were diluted with ispropyl alcohol, extracted, centrifuged, and then the supernatant was transferred into an appropriate vial or plate. Samples were separated on a Waters BEH 50 mm×2.1 mM column, heated to 70° C., using water and acetonitrile mobile phases with a flow rate of 0.5 mL/min. The gradient consisted of the following steps: 0 minutes 0% B, 1 minutes 99% B, 2 minutes 99% B, and 2.1 minutes 0% B. The gradient used a linear ramp for all transitions, and the method was 3 minutes long. A specific MRM was used to detect bakuchiol in the mass spectrometry with an ESI source in the negative ion mode: Parent 255.2 m/z (unit), Product 172.1 m/z, Fragmenter 120V, Collision Energy 20 V, Cell Accelerator Voltage 5V with a 300 msec dwell time. Optical detection was also conducted at 260 nm with a 0.5 sec response time.

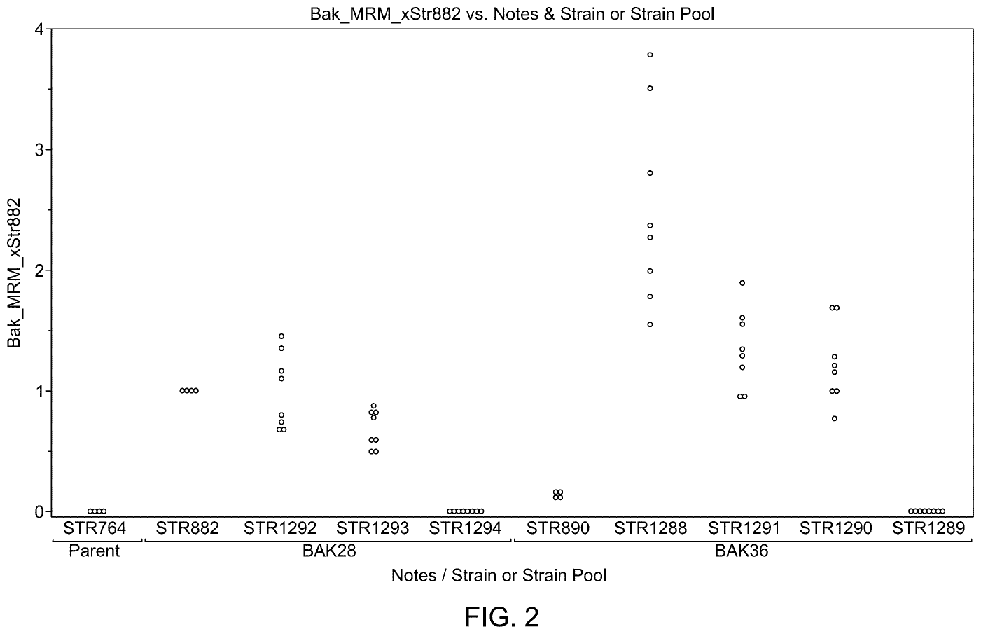

Of the N-terminal truncation variants tested, BAK36(T1) increased bakuchiol titers 18-fold over parent (FIG. 2).

In order to further optimize bakuchiol production by BAK36(T1), complete saturation mutagenesis was performed on BAK36(T1) by designing an Inscripta Onyx library of about 7,100 members. Approximately 10,000 clonal samples were screened in singlicate using the plate assay previously described above. Significant hits above parent were singulated and four biological replicates were re-screened to validate each hit. A subset of samples that showed loss of titer (strikes), were also re-screened in duplicate. Validated hits and strikes were sequenced via next generation sequencing (NGS) and analyzed for both barcode and presence of edit. Sequencing analysis resulted in 48 unique hits at 26 amino acid positions (Table 2) and 149 unique strikes at 79 amino acids, with some residues having multiple amino acid substitutions resulting in phenotype. Table 2 summarizes the unique hits; the amino acid residue change shows the change relative to the corresponding amino acid position in SEQ ID NO: 3.

TABLE 2
BAK36 (T1) Unique Hits

| | HTS Plate Titer- | HTS Plate |
|---|---|---|
| AA | fold over parent | Titer - CV % |
| E54F | 1.42 | 15.31% |
| G71D | 1.56 | 7.56% |
| G71D | 1.29 | 16.50% |
| S108L | 1.78 | 20.05% |
| T162H | 1.87 | 4.64% |
| P185V | 1.33 | 7.63% |
| P185V | 1.55 | 15.85% |
| V199G | 6.23 | 8.42% |
| P205L | 14.8 | 4.67% |
| P205V | 1.2 | 9.96% |
| L206Y | [NO DATA | [NO DATA |
| | PROVIDED] | PROVIDED] |
| W209S | 13.32 | 2.40% |
| W209C | 24.18 | 7.73% |
| W209S | 11.85 | 7.84% |
| W209V | 14.8 | 4.67% |
| W209T | 10.92 | 8.51% |
| W209Y | 5.56 | 4.87% |
| W209Y | 5.96 | 2.70% |
| W209Y | 4.66 | 6.94% |
| W209R | 5.93 | 2.08% |
| W209M | 4.83 | 41.84% |
| W209Q | 1.33 | 9.69% |
| L226M | 1.33 | 3.36% |
| L234Q | 1.37 | 8.78% |
| F257E | 1.34 | 13.22% |
| K269R | 1.38 | 7.02% |
| I274L | 1.4 | 14.01% |
| I274L | 1.35 | 11.78% |
| I274L | 1.26 | 10.40% |
| D279C | 1.49 | 6.04% |
| D279K | 1.73 | 7.98% |
| D279R | 1.63 | 11.28% |
| D279R | 1.47 | 5.37% |
| D279M | 1.55 | 19.86% |
| D279L | 1.67 | 6.96% |
| D279L | 1.78 | 7.56% |
| D279L | 1.42 | 6.82% |
| M287V | 2.65 | 5.80% |
| M287F | 1.46 | 9.55% |
| M287F | 1.43 | 6.71% |
| M287Y | 1.4 | 9.25% |
| I310V | 1.5 | 6.24% |
| V312W | 1.55 | 3.35% |
| V312W | 1.38 | 4.33% |
| V312W | 1.42 | 9.69% |
| V312W | 1.3 | 7.23% |
| V312W | 1.3 | 14.92% |
| V312A | 1.33 | 8.29% |
| V312F | 1.19 | 3.24% |
| V312F | 1.48 | 7.77% |
| V312F | 1.42 | 17.54% |
| V312G | 1.37 | 19.67% |
| V312G | 1.49 | 7.30% |
| V312Y | 1.44 | 9.98% |
| V312Y | 1.79 | 18.41% |
| V312Y | 1.4 | 18.32% |
| V312Y | 1.69 | 5.73% |
| V312Y | 1.67 | 13.79% |
| V312Y | 1.57 | 11.21% |
| V312C | 1.32 | 8.75% |
| V312L | 1.51 | n/a |
| G313I | 1.38 | 7.02% |
| S317P | 1.37 | 19.27% |
| S317I | 1.49 | 7.22% |
| F318R | 1.65 | 15.18% |
| F318R | 1.53 | 6.19% |
| F318R | 1.46 | 8.56% |
| F318G | 1.22 | 5.27% |
| L319P | 1.34 | 22.30% |
| W320D | 1.26 | 6.27% |
| T325G | 1.37 | 7.18% |
| S342G | 1.84 | 2.48% |
| L354F | 1.68 | 4.41% |

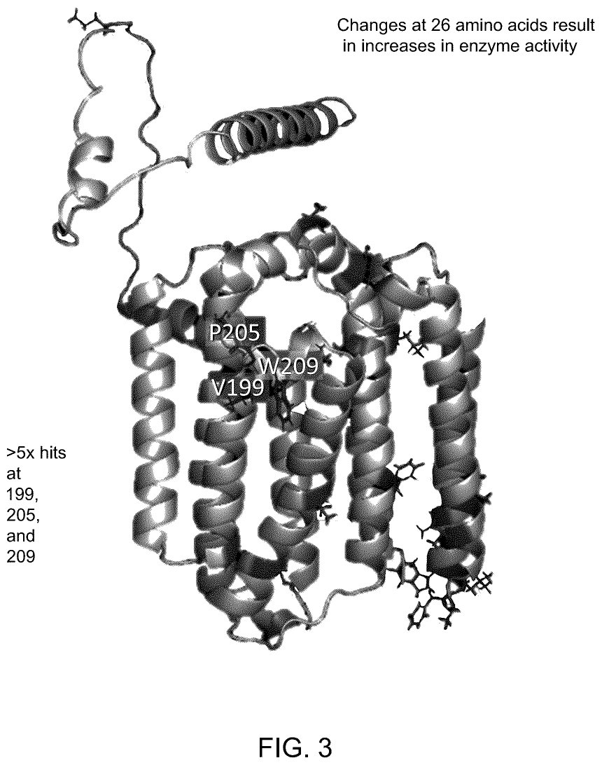

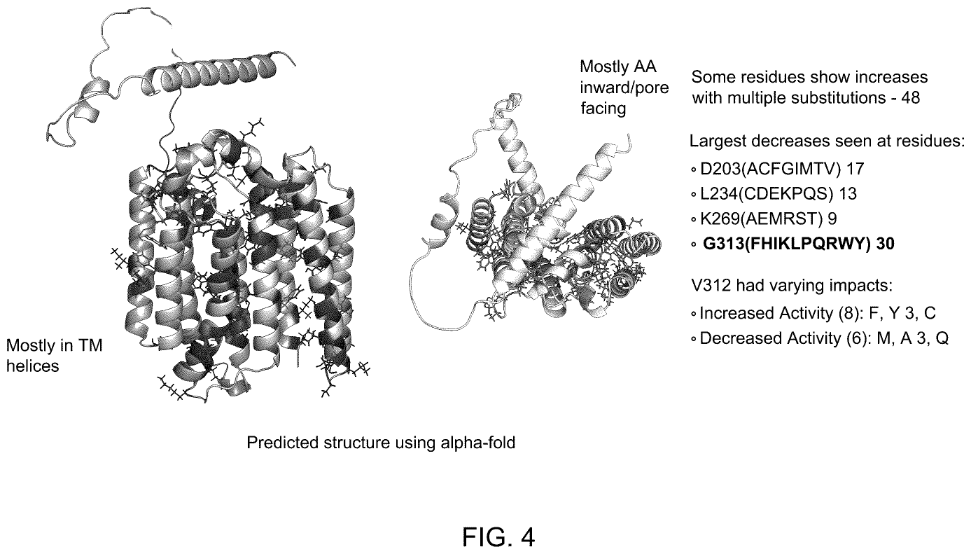

A predicted structure of BAK36(T1) was generated using AlphaFold. The resulting structure showed 9 transmembrane (TM) regions as predicted by TOPCONS. Most of the substitutions that resulted in BAK36(T1) improvement were predicted to lie on internally or externally facing loops and not in the TM helices (FIG. 3). Substitutions at three amino acids (V199, P205 and W209), resulted in greater than 5-fold improvement (residues colored in pink; FIG. 3) with W209C and P205L resulting in 24-fold and 15-fold improvements, respectively (Table 3). In contrast, many of the residues shown to decrease BAK36(T1) function were found to be in the TM helices or residues with side chains facing inward toward the enzyme pores (FIG. 4). The most common strikes were at residues D203, L234, K269 and G313. Several BAK36 (T1) residues had multiple amino acid substitutions that resulted in both improvement and decreases in bakuchiol production. For example the mutations V312F, V312Y, and V312C resulted in increased bakuchiol titer, while the mutations V312M, V312A, and V312Qresulted in decreased bakuchiol titers relative to the parent strain.

Adding some of the largest single amino acid substitution hits (W209C and W209S) to our optimized strain lineages, resulted in increased production of bakuchiol in numerous genotypic contexts. This result confirmed that a bakuchiol-producing enzyme could be engineered to produce, when expressed in a microbial cell, much higher levels of bakuchiol than achieved previously. Such an engineered enzyme may be useful for large scale bioproduction of bakuchiol.

### II. Example 2—Enhancement of Bakuchiol Production with Heterologous Chaperone Proteins

The addition of an ER1, PM1, PX1, or VC1 tag to the C-terminus of BAK36 (T1) resulted in enzymes that achieve bakuchiol titers substantially lower than that of BAK36 (T1). The amino acid sequences of the appended C-terminal tags are set forth in Table 3.

TABLE 3
Amino Acid Sequence of tested C-Terminal Tags
appended to BAK36 (T1)

| | | SEQ |
|---|---|---|
| | | ID |
| Tag | Amino Acid Sequence | NO: |
| ER1 | MTNASSNKTEKIKHEYANMRHRQHNLKHNYGGIEG | 56 |
| | VSTCEDWARNFVVNAASGESLESHEAQHHTPETLWG | |
| | SIKQFCDAFYRFSRPHVIIGTAVNIIVMSSLALEKSSDI | |
| | SPKFFIGLFQVIVTILSMNIYTAGINQLTDIEIDKINKPY | |
| | LPLASGEYSYKTGVTIITLCAILSLGVGWIVGSPPLFW | |
| | SNFAYFVLGTVYSIDLPLMRWKSHPALAALFFFVIRG | |
| | LTFHVGFFLHLQTHVFKRPMMIPKSVMFGTAFMSFF | |
| | YVIIAFFKDIPDIEGDKDHGVKSLTMRLGQERVFWIC | |
| | VSLLLTGYGAAIVVGATSSFLWCKLITVSGHALLASIF | |
| | WNRAKSVDLKSHQEITSLYMFMWKLFYAEYFIIPLM | |
| | RILEQPLKFVLTAAVVLLTTSVLCCVVFT* | |
| PM1 | MTNASSNKTEKIKHEYANMRHRQHNLKHNYGGIEG | 57 |
| | VSTCEDWARNFVVNAASGESLESHEAQHHTPETLWG | |
| | SIKQFCDAFYRFSRPHVIIGTAVNIIVMSSLALEKSSDI | |
| | SPKFFIGLFQVIVTILSMNIYTAGINQLTDIEIDKINKPY | |
| | LPLASGEYSYKTGVTIITLCAILSLGVGWIVGSPPLFW | |
| | SNFAYFVLGTVYSIDLPLMRWKSHPALAALFFFVIRG | |
| | LTFHVGFFLHLQTHVFKRPMMIPKSVMFGTAFMSFF | |
| | YVIIAFFKDIPDIEGDKDHGVKSLTMRLGQERVFWIC | |
| | VSLLLTGYGAAIVVGATSSFLWCKLITVSGHALLASIF | |
| | WNRAKSVDLKSHQEITSLYMFMWKLFYAEYFIIPLM | |
| | RWYKDLKMKMCLALVIIILLVVIIVPIAVHFSR* | |
| PX1 | MTNASSNKTEKIKHEYANMRHRQHNLKHNYGGIEG | 58 |
| | VSTCEDWARNFVVNAASGESLESHEAQHHTPETLWG | |
| | SIKQFCDAFYRFSRPHVIIGTAVNIIVMSSLALEKSSDI | |
| | SPKFFIGLFQVIVTILSMNIYTAGINQLTDIEIDKINKPY | |
| | LPLASGEYSYKTGVTIITLCAILSLGVGWIVGSPPLFW | |
| | SNFAYFVLGTVYSIDLPLMRWKSHPALAALFFFVIRG | |
| | LTFHVGFFLHLQTHVFKRPMMIPKSVMFGTAFMSFF | |
| | YVIIAFFKDIPDIEGDKDHGVKSLTMRLGQERVFWIC | |
| | VSLLLTGYGAAIVVGATSSFLWCKLITVSGHALLASIF | |
| | WNRAKSVDLKSHQEITSLYMFMWKLFYAEYFIIPLM | |
| | RSKL* | |
| VC1 | MTNASSNKTEKIKHEYANMRHRQHNLKHNYGGIEG | 59 |
| | VSTCEDWARNFVVNAASGESLESHEAQHHTPETLWG | |
| | SIKQFCDAFYRFSRPHVIIGTAVNIIVMSSLALEKSSDI | |
| | SPKFFIGLFQVIVTILSMNIYTAGINQLTDIEIDKINKPY | |
| | LPLASGEYSYKTGVTIITLCAILSLGVGWIVGSPPLFW | |
| | SNFAYFVLGTVYSIDLPLMRWKSHPALAALFFFVIRG | |
| | LTFHVGFFLHLQTHVFKRPMMIPKSVMFGTAFMSFF | |
| | YVIIAFFKDIPDIEGDKDHGVKSLTMRLGQERVFWIC | |
| | VSLLLTGYGAAIVVGATSSFLWCKLITVSGHALLASIF | |
| | WNRAKSVDLKSHQEITSLYMFMWKLFYAEYFIIPLM | |
| | RNIKEIMWWQKVKNITLLTFTIILFVSAAFMFFYLW* | |

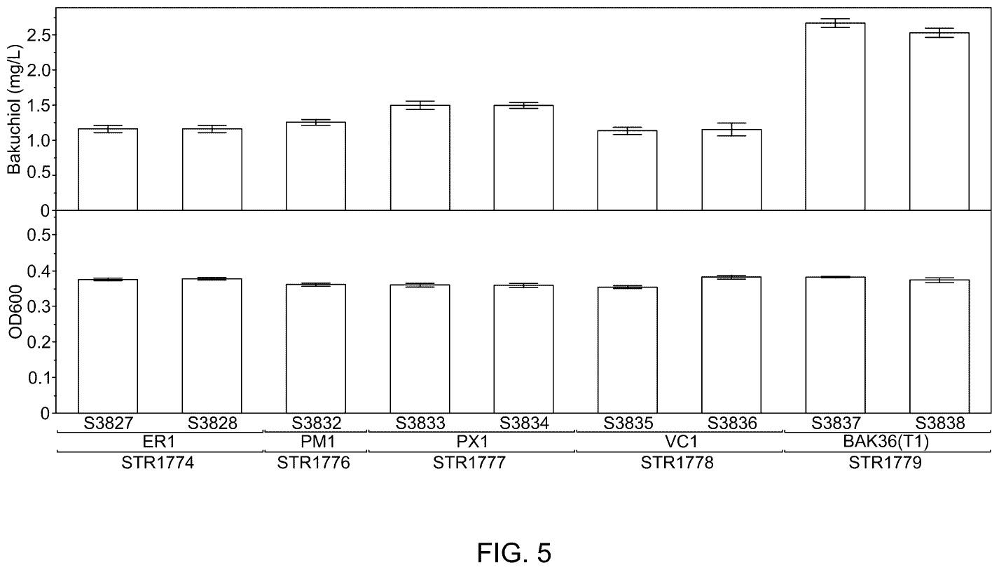

Attempts to improve the BAK36 enzyme by adding C-terminal tags consistently resulted in a decrease in enzyme activity, regardless of the size of the tag (FIG. 5), which suggests that alterations or additions at the C-terminus of the enzyme are unlikely to increase enzymatic activity. Indeed, BAK36(T1) consistently produced higher levels of bakuchiol than each C-terminally tagged enzyme tested. Given the extreme sensitivity of the C-terminus of BAK36(T1) to perturbation, it is likely that the C-terminus of BAK36(T1) is important for catalyzing the production of bakuchiol.

It is unclear whether the decrease in activity was a result of misfolding of the enzyme, but that was one possibility. Heterologous expression of enzymes can result in misfolding or deviations from the natural folding pattern, which is required for optimal enzymatic activity. Accordingly, interventions that promote proper protein folding are advantageous to producing enzymes with optimal activity in cells.

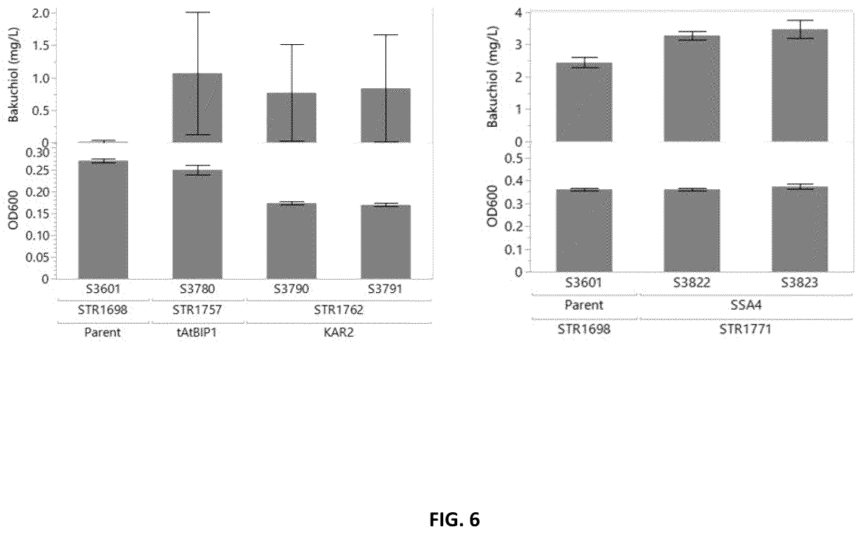

In order to increase accurate protein folding, a library of heterologous and native chaperones was generated and screened for improved bakuchiol titers. Library members were co-expressed in yeast cells together with BAK36 (T1), and bakuchiol titers were measured as discussed above. Three out of 18 chaperones showed increases in titers, which suggests improved protein folding by the following three chaperones: truncated Arabidopsis thaliana BIP1 (tAtBIP1) (SEQ ID NO: 33), SSA4 (SEQ ID NO: 34) and KAR2 (SEQ ID NO: 35) (FIG. 6). This result showed that production of bakuchiol by a bakuchiol-producing enzyme could be improved (i.e., increased) by co-expression of a heterologous chaperone protein in a microbial cell. Engineered microbial cells expressing an engineered bakuchiol-producing protein and a heterologous chaperone protein may be useful for the large scale bioproduction of bakuchiol.

Thus, the experiments in this example showed that co-expression in yeast of certain heterologous chaperones (e.g., truncated Arabidopsis thaliana BIP1 (tAtBIP1) (SEQ ID NO: 33), SSA4 (SEQ ID NO: 34) and KAR2 (SEQ ID NO: 35)) and other heterologous proteins can increase the likelihood of proper protein folding of the heterologous proteins, and in the case of heterologous enzymes that catalyze bioproduction of a desired product, such co-express can increase titers of the product.

These results showed that one or more bakuchiol genetic pathway manipulations were introduced to a microbial cell expressing a bakuchiol-producing protein to enable increased production of bakuchiol by the microbial cell. These pathway manipulations, and microbial cells comprising the same, may be useful for the large scale bioproduction of bakuchiol.

It should be appreciated that all combinations of the disclosed concepts are provided as being part of the inventive subject matter disclosed herein and may be employed in any combination to achieve the benefits described herein.

The present technology is not to be limited in terms of the particular implementations described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular implementations only, and is not intended to be limiting.

1. An engineered chaperone protein comprising an amino acids sequence in which 2-27 amino acids are deleted from the N-terminus of SEQ ID NO: 60.

2. The engineered chaperone protein of claim 1, wherein the protein comprises an N-terminal deletion of 27 amino acids from SEQ ID NO: 52.

3. The engineered chaperone protein of claim 1, wherein the protein has an amino acid sequence consisting of: SEQ ID NO: 65.

4. (canceled)

5. The engineered chaperone protein of claim 1, wherein the protein exhibits increased protein-folding activity relative to a wild-type form of the protein.

6. An engineered microbial cell expressing a heterologous chaperone protein or variant thereof, wherein the heterologous protein comprises an amino acid sequence that has least about 95% sequence identity to SEQ ID NO: 52, SEQ ID NO: 53, SEQ ID NO: 54, or SEQ ID NO: 55.

7. (canceled)

8. The engineered microbial cell of claim 6, wherein the protein comprises any one of SEQ ID NOs: 52, 53, 54, or 55.

9. The engineered microbial cell of claim 6, wherein the protein or variant thereof is a variant of SEQ ID NO: 1 comprising an N-terminal deletion of 1-27 amino acids from SEQ ID NO: 52.

10-12. (canceled)

13. The engineered microbial cell of claim 6, wherein the microbial cell is a yeast cell or a bacterial cell.

14. The engineered microbial cell of claim 6, wherein the microbial cell expresses a second heterologous protein, wherein the second heterologous protein is an enzym.

15. (canceled)

16. The engineered microbial cell of claim 14, wherein the enzyme catalyzes production of bakuchiol, exhibits prenyltransferase activity, or both.

17. A method of expressing a heterologous protein in a microbial cell, comprising co-expressing the heterologous protein and a heterologous chaperone protein.

18. The method of claim 17, wherein the microbial cell is a yeast cell or a bacterial cell.

19. The method of claim 17, wherein the heterologous chaperone protein is a chaperone protein from Arabidopsis thaliana.

20. The method of claim 17, wherein the heterologous chaperone protein is Arabidopsis thaliana BIP1 (AtBIP1) or a variant thereof.

21. The method of claim 17, wherein the heterologous chaperone protein comprises an amino acid sequence that has at least about 95% sequence identity to SEQ ID NO: 52 or SEQ ID NO: 53.

22. The method of claim 17, wherein the heterologous chaperone protein comprises SEQ ID NO: 52, is a variant of SEQ ID NO: 32 comprising an N-terminal deletion of 1-27 amino acids from SEQ ID NO: 52, or consists of SEQ ID NO: 53.

23-24. (canceled)

25. The method of claim 17, wherein the heterologous chaperone protein comprises an amino acid sequence that has at least about 95% sequence identity to SEQ ID NO: 54 or SEQ ID NO: 55.

26. The method of claim 25, wherein the heterologous chaperone protein comprises SEQ ID NO: 54 or SEQ ID NO: 55.

27. (canceled)

28. The method of claim 17, wherein the heterologous protein is an enzyme, wherein the enzyme catalyzes production of bakuchiol, exhibits prenyltransferase activity, or both.

29. (canceled)

30. The method of claim 17, wherein expression of the heterologous protein is increased relative to expression of the heterologous protein in a microbial cell that does not co-express the heterologous chaperone protein.

31-41. (canceled)
